(19) United States
(12) Patent Application Publication
(10) Pub. No.: US 2025/0383355 A1
(43) Pub. Date: Dec. 18, 2025

(54) BIOMARKERS FOR ENDOMETRIAL CANCER

(71) Applicants: FUNDACIÓ HOSPITAL UNIVERSITARI VALL D'HEBRON - INSTITUT DE RECERCA, BARCELONA (ES); LUXEMBOURG INSTITUTE OF HEALTH (LIH), LUXEMBOURG (LU)

(72) Inventors: EVA COLL DE LA RUBIA, BARCELONA (ES); EVA COLAS ORTEGA, BARCELONA (ES); SILVIA CABRERA DÍAZ, BARCELONA (ES); ANTONIO GIL MORENO, BARCELONA (ES); ELENA MARTINEZ GARCIA, LUXEMBOURG (LU); GUNNAR ALFRED GÜNTHER DITTMAR, ESCH-SUR-ALZETTE (LU)

(21) Appl. No.: 18/291,071

(22) PCT Filed: Jul. 22, 2022
(86) PCT No.: PCT/EP2022/070680

§ 371 Date: Jan. 22, 2024

(30) Foreign Application Priority Data
Jul. 23, 2021 (EP) 21382680.3

(51) Int. Cl. G01N 33/574; G01N 33/68

(52) U.S. Cl. CPC G01N 33/57442; G01N 33/57484; G01N 33/6872

(57) ABSTRACT

A method for diagnosing and prognosing endometrial cancer in easy-to-access isolated gynecological samples by detecting the level of expression of one or more proteins. In particular from fluid samples of the female genital tract.

This is a National Stage application of PCT international application PCT/EP2022/070680, filed on Jul. 22, 2022, which claims the benefit of European Patent Application EP21382680 filed on Jul. 23, 2021, both of which are incorporated herein by reference in their entirety.

### TECHNICAL FIELD

The invention relates to the diagnosis and prognosis of endometrial cancer.

### BACKGROUND ART

Endometrial cancer (EC) is the most frequently observed invasive tumor of the female genital tract and the fourth most common cancer in women in developed countries, accounting for 66,570 diagnosed cases and 12,940 estimated deaths in 2021 in the

United States. Its early diagnosis is associated with 95% of 5-year survival rate. However, when it is diagnosed at advanced stages, the 5-year survival rate decreases dramatically to 69% in case of local metastasis and 16% in those cases with distant metastasis. Currently, there are no screening tools for its early diagnosis, and the diagnostic process starts with the observation of related symptoms, being abnormal vaginal bleeding (AVB) the most common. Even though 90% of EC patients will experience AVB, this symptom is not specific of the disease and only a 9% of the studied patients will finally present EC. The first diagnostic step is to perform the pathological evaluation of an endometrial pipelle biopsy. However, this procedure is associated to 22% of failure and these patients will undergo additional invasive procedures such as hysteroscopy to be diagnosed. Yearly, ˜7M women experience AVB in Europe and begin this diagnostic process, causing morbidity to patients and a big burden to the healthcare systems. Improving early diagnosis is hence a major issue to appropriately manage EC and decrease mortality associated to the disease. Discrimination of patients with benign endometrial pathologies and with EC is only achieved after a tedious diagnostic process consisting of a pelvic examination and transvaginal ultrasonography followed by a confirmatory histopathological examination of an endometrial biopsy. The preferable biopsy used in this procedure is named uterine aspirate and/or pipelle biopsy and is obtained by a minimally invasive aspiration of endometrial fluid from inside the uterine cavity. Because the current diagnostic procedures on uterine aspirates rely on the presence of cellular material, this process has unfortunately a diagnostic failure and an associated inadequate sampling rate of 8% and 15%, respectively. This is increased in postmenopausal women up to 12% and 22%. In those cases, a biopsy guided by hysteroscopy needs to be performed, where this invasive technique presents an increased risk of complications, including uterine perforation, hemorrhage and possible harm to other organs.

The European patent EP3452829B1, and the European patent application EP3655778A1 disclose useful markers that can be determined in the uterine aspirate and that provide good specificities and sensitivities for the differential diagnosis of EC from other endometrial conditions. The document EP3655778A1 provides a method for the prognosis of EC, for being able to distinguish among two of the subtypes of EC; endometroid endometrial cancer (EEC) from non-endometrioid EC cases (NEEC).

To date, many studies have also been conducted to identify EC protein biomarkers, mainly in tissue and serum samples (see for example DeSouza L V, et al, “Endometrial cancer biomarker discovery and verification using differentially tagged clinical samples with multidimensional liquid chromatography and tandem mass spectrometry”, Mol Cell Proteomics MCP—2007, vol. no.6, pp.: 1170-8, or Kemik P, et al. “Diagnostic and prognostic values of preoperative serum levels of YKL-40, HE-4 and DKK-3 in endometrial cancer”, Gynecol Oncol—2016; vol. no. 140, pp.: 64-9). None of them have been translated into clinical utility.

Other documents disclosing plasma or serum samples for retrieving important information about EC lead to controversial or contradictory conclusions. For example, whilst the document of Tanable et al., “Midkine and its clinical significance in endometrial carcinoma”, cancer Sci—2008, vol. no. 99 (6), pp.: 1125-1130, proposes midkine (MDK), a secreted heparin-binding growth factor, as useful serum biomarker for identifying high risk patients of EC; the document of Torres et al., “CD44, TGM2 and EpCAM as novel plasma markers in endometrial cancer diagnosis”, BMC Cancer—2019 19:401 https://doi.org/10.1186/s12885-019-5556-x. (see FIG. 2(f)), identified that MDK is not able to distinguish EC from healthy controls if the endometriosis patients are taken out of the endometrial cancer subgroup. Indeed, Torres et al. conclude that plasma markers like TGM2 can accurately diagnose EC, but others, such as MDK, might be altered in EC studies by the inclusion of endometriosis cases, and this need to be taken in consideration in future research design. Finally, MDK has also been identified in tissue biopsies from the cervix and giving information about cervical cancer in the document of Moon et al., “Immunohistochemical and quantitative competitive PCR analyses of midkine and pleiotrophin expression in cervical cancer”, Gynecologic Oncology—2003, vol. no. 88, pp.: 289-297.

In any case, of note is that some of the studied samples (tissue biopsies, plasma and serum) are non-routine gynecological samples, and all of them (including uterine aspirates) are minimally-invasive, which precludes their use as an easy-access screening and/or diagnostic tool.

Concluding, despite the efforts made, there is still the need of a trustable rule out method to reduce the current burden of women entering through the diagnostic process, as well as highly accurate biomarkers that can be assessed easily in a clinical environment obtained from non-invasive samples to improve the diagnosis, and even the prognosis of endometrial cancer.

### SUMMARY OF INVENTION

Inventors have determined that certain protein markers detectable in isolated samples obtainable from methods used in the regular or routine controls of gynecology, give valuable diagnostic information in endometrial cancer (EC). The proteins were first analysed in a retrospective way from a cohort of 60 patients, including control, EC and patients with cervical pathology. Then, the group of informative proteins was retrospectively validated in a larger cohort of 242 patients (106 non-EC, 129 EC, and 7 premalignant lessions of EC, I.e. hyperplasias).

In addition, inventors have determined that some proteins also detectable in these types of samples, are meaningful prognostic biomarkers of endometrial cancer (EC). These proteins allow discrimination between EC presenting different prognosis, including different histological subtypes and grades and different molecular features, with high sensitivity and high specificity, and thus they allow minimizing the risk of false positive and false negative classification among these subtypes.

Thus, in a first aspect, the invention relates to a method of diagnosis and/or for the prognosis of EC, the method comprising determining the presence and/or the level of expression of midkine (MDK) in a sample from the female genital tract part including one or more of the vulvae, vagina, cervix, uterus, fallopian tubes, and ovaries and selected from a gynecologic sampling, including or selected from a cervical fluid, a cytology, a pap-smear sample, an endometrial biopsy, a uterine fluid, uterine washings and combinations thereof.

MDK is (MK or MDK), also known as neurite growth-promoting factor 2 (NEGF2). It is a protein that in humans is encoded by the MDK gene. Midkine is a basic heparin-binding growth factor of low molecular weight, and forms a family with pleiotrophin (NEGF1, 46% homologous with MK). It is a nonglycosylated protein, composed of two domains held by disulfide bridges. It is a developmentally important retinoic acid-responsive gene product strongly induced during mid-gestation, hence the name midkine. Restricted mainly to certain tissues in the normal adult, it is strongly induced during oncogenesis, inflammation and tissue repair. The canonical amino acid sequence (Isoform 1) has a length of 143 amino acids, at it is identified un the UniProtKB database with the accession number P21741 (version of May 1, 1991 of the sequence and release 189 of the UniProtKB/Swiss-Prot database in Apr. 7, 2021).

Another aspect of the invention is the use of MDK, as in vitro marker for the diagnosis and/or for the prognosis of endometrial cancer in a sample from the female genital tract part including one or more of the vulvae, vagina, cervix, uterus, fallopian tubes, and ovaries and selected from a gynecologic sampling, including a cervical fluid, a cytology, a pap-smear like sample containing cervical fluid, a pap-smear sample, an endometrial biopsy, a uterine fluid, uterine washings and combinations thereof.

In a third aspect, the invention proposes new kits comprising a solid support and means for detecting the presence and/or for determining the level of expression of MDK and optionally means for detecting the presence and/or for determining the level of expression of one or more proteins selected from the group consisting of Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A) and Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Agrin (AGR), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1 (LGALS1), ATPase H+ Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), and Exportin-2 (XPO2).

In other words, this aspect can also be defined as new kits comprising a solid support and means for detecting the presence and/or for determining the level of expression of MDK and optionally means for detecting the presence and/or for determining the level of expression of one or more proteins selected from the group listed in Table 1 below, indicating the entry (accession number) in the UniProtKB/Swiss-Prot database of the release number accesible 23 Jul. 2021 in https://www.uniprot.org/help/uniprotkb:

TABLE 1
Informative proteins for EC.

| Gene names | Entry | Entry name | Protein names |
|---|---|---|---|
| MDK MK1 | P21741 | MK_HUMAN | Midkine (MK) (Amphiregulin-associated |
| NEGF2 | | | protein) (ARAP) (Midgestation and kidney |
| | | | protein) (Neurite outgrowth-promoting factor |
| | | | 2) (Neurite outgrowth-promoting protein) |
| COL1A1 | P02452 | CO1A1_HUMAN | Collagen alpha-1(I) chain (Alpha-1 type I |
| | | | collagen) |
| VWF F8VWF | P04275 | VWF_HUMAN | von Willebrand factor (vWF) [Cleaved into: |
| | | | von Willebrand antigen 2 (von Willebrand |
| | | | antigen II)] |
| APOB | P04114 | APOB_HUMAN | Apolipoprotein B-100 (Apo B-100) [Cleaved |
| | | | into: Apolipoprotein B-48 (Apo B-48)] |
| C1QA | P02745 | C1QA_HUMAN | Complement C1q subcomponent subunit A |
| FN1 FN | P02751 | FINC_HUMAN | Fibronectin (FN) (Cold-insoluble globulin) |
| | | | (CIG) [Cleaved into: Anastellin; Ugl-Y1; Ugl- |
| | | | Y2; Ugl-Y3] |
| SERPIND1 | P05546 | HEP2_HUMAN | Heparin cofactor 2 (Heparin cofactor II) (HC- |
| HCF2 | | | II) (Protease inhibitor leuserpin-2) (HLS2) |
| | | | (Serpin D1) |
| SPP1 BNSP | P10451 | OSTP_HUMAN | Osteopontin (Bone sialoprotein 1) |
| OPN PSEC0156 | | | (Nephropontin) (Secreted phosphoprotein 1) |
| | | | (SPP-1) (Urinary stone protein) (Uropontin) |
| APP A4 AD1 | P05067 | A4_HUMAN | Amyloid-beta precursor protein (APP) (ABPP) |
| | | | (APPI) (Alzheimer disease amyloid protein) |
| | | | (Amyloid precursor protein) (Amyloid-beta A4 |
| | | | protein) (Cerebral vascular amyloid peptide) |
| | | | (CVAP) (PreA4) (Protease nexin-II) (PN-II) |
| | | | [Cleaved into: N-APP; Soluble APP-alpha (S- |
| | | | APP-alpha); Soluble APP-beta (S-APP-beta); |
| | | | C99 (Beta-secretase C-terminal fragment) |
| | | | (Beta-CTF); Amyloid-beta protein 42 |
| | | | (Abeta42) (Beta-APP42); Amyloid-beta |
| | | | protein 40 (Abeta40) (Beta-APP40); C83 |
| | | | (Alpha-secretase C-terminal fragment) |
| | | | (Alpha-CTF); P3(42); P3(40); C80; Gamma- |
| | | | secretase C-terminal fragment 59 (Amyloid |
| | | | intracellular domain 59) (AICD-59) (AID(59)) |
| | | | (Gamma-CTF(59)); Gamma-secretase C- |
| | | | terminal fragment 57 (Amyloid intracellular |
| | | | domain 57) (AICD-57) (AID(57)) (Gamma- |
| | | | CTF(57)); Gamma-secretase C-terminal |
| | | | fragment 50 (Amyloid intracellular domain 50) |
| | | | (AICD-50) (AID(50)) (Gamma-CTF(50)); C31] |
| FGFBP1 FGFBP | Q14512 | FGFP1_HUMAN | Fibroblast growth factor-binding protein 1 |
| HBP17 | | | (FGF-BP) (FGF-BP1) (FGF-binding protein 1) |
| | | | (FGFBP-1) (17 kDa heparin-binding growth |
| | | | factor-binding protein) (17 kDa HBGF-binding |
| | | | protein) (HBp17) |
| TCP1 CCT1 | P17987 | TCPA_HUMAN | T-complex protein 1 subunit alpha (TCP-1- |
| CCTA | | | alpha) (CCT-alpha) |
| APOC1 | P02654 | APOC1_HUMAN | Apolipoprotein C-I (Apo-CI) (ApoC-I) |
| | | | (Apolipoprotein C1) [Cleaved into: Truncated |
| | | | apolipoprotein C-I] |
| APOF | Q13790 | APOF_HUMAN | Apolipoprotein F (Apo-F) (Lipid transfer |
| | | | inhibitor protein) (LTIP) |
| CCT6A CCT6 | P40227 | TCPZ_HUMAN | T-complex protein 1 subunit zeta (TCP-1- |
| CCTZ | | | zeta) (Acute morphine dependence-related |
| | | | protein 2) (CCT-zeta-1) (HTR3) (Tcp20) |
| LBP | P18428 | LBP_HUMAN | Lipopolysaccharide-binding protein (LBP) |
| COL12A1 | Q99715 | COCA1_HUMAN | Collagen alpha-1(XII) chain |
| COL12A1L | | | |
| SAA4 CSAA | P35542 | SAA4_HUMAN | Serum amyloid A-4 protein (Constitutively |
| | | | expressed serum amyloid A protein) (C-SAA) |
| ITIH2 IGHEP2 | P19823 | ITIH2_HUMAN | Inter-alpha-trypsin inhibitor heavy chain H2 |
| | | | (ITI heavy chain H2) (ITI-HC2) (Inter-alpha- |
| | | | inhibitor heavy chain 2) (Inter-alpha-trypsin |
| | | | inhibitor complex component II) (Serum- |
| | | | derived hyaluronan-associated protein) (SHAP) |
| LCAT | P04180 | LCAT_HUMAN | Phosphatidylcholine-sterol acyltransferase |
| | | | (EC 2.3.1.43) (1-alkyl-2- |
| | | | acetylglycerophosphocholine esterase) (EC |
| | | | 3.1.1.47) (Lecithin-cholesterol |
| | | | acyltransferase) (Phospholipid-cholesterol |
| | | | acyltransferase) (Platelet-activating factor |
| | | | acetylhydrolase) (PAF acetylhydrolase) |
| LCN2 HNL | P80188 | NGAL_HUMAN | Neutrophil gelatinase-associated lipocalin |
| NGAL | | | (NGAL) (25 kDa alpha-2-microglobulin- |
| | | | related subunit of MMP-9) (Lipocalin-2) |
| | | | (Oncogene 24p3) (Siderocalin) (p25) |
| MUC4 | Q99102 | MUC4_HUMAN | Mucin-4 (MUC-4) (Ascites sialoglycoprotein) |
| | | | (ASGP) (Pancreatic adenocarcinoma mucin) |
| | | | (Testis mucin) (Tracheobronchial mucin) |
| | | | [Cleaved into: Mucin-4 alpha chain (Ascites |
| | | | sialoglycoprotein 1) (ASGP-1); Mucin-4 beta |
| | | | chain (Ascites sialoglycoprotein 2) (ASGP-2)] |
| C1R | P00736 | C1R_HUMAN | Complement C1r subcomponent (EC |
| | | | 3.4.21.41) (Complement component 1 |
| | | | subcomponent r) [Cleaved into: Complement |
| | | | C1r subcomponent heavy chain; Complement |
| | | | C1r subcomponent light chain] |
| C4BPA C4BP | P04003 | C4BPA_HUMAN | C4b-binding protein alpha chain (C4bp) |
| | | | (Proline-rich protein) (PRP) |
| SPRR1B | P22528 | SPR1B_HUMAN | Cornifin-B (14.9 kDa pancornulin) (Small |
| | | | proline-rich protein IB) (SPR-IB) |
| SERPINH1 CBP1 | P50454 | SERPH_HUMAN | Serpin H1 (47 kDa heat shock protein) |
| CBP2 HSP47 | | | (Arsenic-transactivated protein 3) (AsTP3) |
| SERPINH2 | | | (Cell proliferation-inducing gene 14 protein) |
| PIG14 | | | (Collagen-binding protein) (Colligin) |
| | | | (Rheumatoid arthritis-related antigen RA-A47) |
| LMO7 FBX20 | Q8WWI1 | LMO7_HUMAN | LIM domain only protein 7 (LMO-7) (F-box |
| FBXO20 | | | only protein 20) (LOMP) |
| KIAA0858 | | | |
| S100A9 CAGB | P06702 | S10A9_HUMAN | Protein S100-A9 (Calgranulin-B) (Calprotectin |
| CFAG MRP14 | | | L1H subunit) (Leukocyte L1 complex heavy |
| | | | chain) (Migration inhibitory factor-related |
| | | | protein 14) (MRP-14) (p14) (S100 calcium- |
| | | | binding protein A9) |
| SPRR1A | P35321 | SPR1A_HUMAN | Cornifin-A (19 kDa pancornulin) (SPRK) |
| | | | (Small proline-rich protein IA) (SPR-IA) |
| AGRN AGRIN | O00468 | AGRIN_HUMAN | Agrin [Cleaved into: Agrin N-terminal 110 kDa |
| | | | subunit; Agrin C-terminal 110 kDa subunit; |
| | | | Agrin C-terminal 90 kDa fragment (C90); |
| | | | Agrin C-terminal 22 kDa fragment (C22)] |
| TIMP2 | P16035 | TIMP2_HUMAN | Metalloproteinase inhibitor 2 (CSC-21K) |
| | | | (Tissue inhibitor of metalloproteinases 2) |
| | | | (TIMP-2) |
| COL3A1 | P02461 | CO3A1_HUMAN | Collagen alpha-1(III) chain |
| ANXA7 ANX7 | P20073 | ANXA7_HUMAN | Annexin A7 (Annexin VII) (Annexin-7) |
| SNX OK/SW- | | | (Synexin) |
| cl.95 | | | |
| PPL KIAA0568 | O60437 | PEPL_HUMAN | Periplakin (190 kDa paraneoplastic |
| | | | pemphigus antigen) (195 kDa cornified |
| | | | envelope precursor protein) |
| SCEL | O95171 | SCEL_HUMAN | Sciellin |
| IGFALS ALS | P35858 | ALS_HUMAN | Insulin-like growth factor-binding protein |
| | | | complex acid labile subunit (ALS) |
| GPLD1 PIGPLD1 | P80108 | PHLD_HUMAN | Phosphatidylinositol-glycan-specific |
| | | | phospholipase D (PI-G PLD) (EC 3.1.4.50) |
| | | | (Glycoprotein phospholipase D) (Glycosyl- |
| | | | phosphatidylinositol-specific phospholipase |
| | | | D) (GPI-PLD) (GPI-specific phospholipase D) |
| TMPRSS11E | Q9UL52 | TM11E_HUMAN | Transmembrane protease serine 11E (EC |
| DESC1 | | | 3.4.21.-) (Serine protease DESC1) |
| TMPRSS11E2 | | | (Transmembrane protease serine 11E2) |
| UNQ742/ | | | [Cleaved into: Transmembrane protease |
| PRO1461 | | | serine 11E non-catalytic chain; |
| | | | Transmembrane protease serine 11E |
| | | | catalytic chain] |
| BTD | P43251 | BTD_HUMAN | Biotinidase (Biotinase) (EC 3.5.1.12) |
| ANXA3 ANX3 | P12429 | ANXA3_HUMAN | Annexin A3 (35-alpha calcimedin) (Annexin |
| | | | III) (Annexin-3) (Inositol 1,2-cyclic phosphate |
| | | | 2-phosphohydrolase) (Lipocortin III) |
| | | | (Placental anticoagulant protein III) (PAP-III) |
| SERPINA3 AACT | P01011 | AACT_HUMAN | Alpha-1-antichymotrypsin (ACT) (Cell growth- |
| GIG24 GIG25 | | | inhibiting gene 24/25 protein) (Serpin A3) |
| | | | [Cleaved into: Alpha-1-antichymotrypsin His- |
| | | | Pro-less] |
| FCGBP | Q9Y6R7 | FCGBP_HUMAN | IgGFc-binding protein (Fcgamma-binding |
| | | | protein antigen) (FcgammaBP) |
| FAM107B | Q9H098 | F107B_HUMAN | Protein FAM107B |
| C10orf45 | | | |
| STX7 | O15400 | STX7_HUMAN | Syntaxin-7 |
| PLBD1 | Q6P4A8 | PLBL1_HUMAN | Phospholipase B-like 1 (EC 3.1.1.-) (LAMA- |
| | | | like protein 1) (Lamina ancestor homolog 1) |
| | | | (Phospholipase B domain-containing protein |
| | | | 1) [Cleaved into: Phospholipase B-like 1 |
| | | | chain A; Phospholipase B-like 1 chain B; |
| | | | Phospholipase B-like 1 chain C] |
| GRN | P28799 | GRN_HUMAN | Progranulin (PGRN) (Acrogranin) (Epithelin |
| | | | precursor) (Glycoprotein of 88 Kda) (GP88) |
| | | | (Glycoprotein 88) (Granulin precursor) (PC |
| | | | cell-derived growth factor) (PCDGF) |
| | | | (Proepithelin) (PEPI) [Cleaved into: |
| | | | Paragranulin; Granulin-1 (Granulin G); |
| | | | Granulin-2 (Granulin F); Granulin-3 (Epithelin- |
| | | | 2) (Granulin B); Granulin-4 (Epithelin-1) |
| | | | (Granulin A); Granulin-5 (Granulin C); |
| | | | Granulin-6 (Granulin D); Granulin-7 (Granulin E)] |
| IMPA1 IMPA | P29218 | IMPA1_HUMAN | Inositol monophosphatase 1 (IMP 1) (IMPase |
| | | | 1) (EC 3.1.3.25) (D-galactose 1-phosphate |
| | | | phosphatase) (EC 3.1.3.94) (Inositol-1(or 4)- |
| | | | monophosphatase 1) (Lithium-sensitive myo- |
| | | | inositol monophosphatase A1) |
| A2ML1 CPAMD9 | A8K2U0 | A2ML1_HUMAN | Alpha-2-macroglobulin-like protein 1 (C3 and |
| | | | PZP-like alpha-2-macroglobulin domain- |
| | | | containing protein 9) |
| NEDD8 | Q15843 | NEDD8_HUMAN | NEDD8 (Neddylin) (Neural precursor cell |
| | | | expressed developmentally down-regulated |
| | | | protein 8) (NEDD-8) (Ubiquitin-like protein |
| | | | Nedd8) |
| CSE1L CAS | P55060 | XPO2_HUMAN | Exportin-2 (Exp2) (Cellular apoptosis |
| XPO2 | | | susceptibility protein) (Chromosome |
| | | | segregation 1-like protein) (Importin-alpha re- |
| | | | exporter) |
| EPS8L1 DRC3 | Q8TE68 | ES8L1_HUMAN | Epidermal growth factor receptor kinase |
| EPS8R1 | | | substrate 8-like protein 1 (EPS8-like protein |
| PP10566 | | | 1) (Epidermal growth factor receptor pathway |
| | | | substrate 8-related protein 1) (EPS8-related |
| | | | protein 1) |
| VIM | P08670 | VIME_HUMAN | Vimentin |
| CAPS | Q13938 | CAYP1_HUMAN | Calcyphosin (Calcyphosine) |
| CA1 | P00915 | CAH1_HUMAN | Carbonic anhydrase 1 (EC 4.2.1.1) |
| | | | (Carbonate dehydratase I) (Carbonic |
| | | | anhydrase B) (CAB) (Carbonic anhydrase I) |
| | | | (CA-I) |
| KRT8 CYK8 | P05787 | K2C8_HUMAN | Keratin, type II cytoskeletal 8 (Cytokeratin-8) |
| | | | (CK-8) (Keratin-8) (K8) (Type-Il keratin Kb8) |
| PEA15 | Q15121 | PEA15_HUMAN | Astrocytic phosphoprotein PEA-15 (15 kDa |
| | | | phosphoprotein enriched in astrocytes) |
| | | | (Phosphoprotein enriched in diabetes) (PED) |
| CHMP4B | Q9H444 | CHM4B_HUMAN | Charged multivesicular body protein 4b |
| C20orf178 | | | (Chromatin-modifying protein 4b) (CHMP4b) |
| SHAX1 | | | (SNF7 homolog associated with Alix 1) |
| | | | (SNF7-2) (hSnf7-2) (Vacuolar protein sorting- |
| | | | associated protein 32-2) (Vps32-2) (hVps32-2) |
| CTNNB1 CTNNB | P35222 | CTNB1_HUMAN | Catenin beta-1 (Beta-catenin) |
| OK/SW-cl.35 | | | |
| PRO2286 | | | |
| CALU | O43852 | CALU_HUMAN | Calumenin (Crocalbin) (IEF SSP 9302) |
| GYG1 GYG | P46976 | GLYG_HUMAN | Glycogenin-1 (GN-1) (GN1) (EC 2.4.1.186) |
| LGALS1 | P09382 | LEG1_HUMAN | Galectin-1 (Gal-1) (14 kDa laminin-binding |
| | | | protein) (HLBP14) (14 kDa lectin) (Beta- |
| | | | galactoside-binding lectin L-14-I) (Galaptin) |
| | | | (HBL) (HPL) (Lactose-binding lectin 1) (Lectin |
| | | | galactoside-binding soluble 1) (Putative |
| | | | MAPK-activating protein PM12) (S-Lac lectin 1) |
| PKM OIP3 PK2 | P14618 | KPYM_HUMAN | Pyruvate kinase PKM (EC 2.7.1.40) |
| PK3 PKM2 | | | (Cytosolic thyroid hormone-binding protein) |
| | | | (CTHBP) (Opa-interacting protein 3) (OIP-3) |
| | | | (Pyruvate kinase 2/3) (Pyruvate kinase |
| | | | muscle isozyme) (Thyroid hormone-binding |
| | | | protein 1) (THBP1) (Tumor M2-PK) (p58) |
| HCLS1 HS1 | P14317 | HCLS1_HUMAN | Hematopoietic lineage cell-specific protein |
| | | | (Hematopoietic cell-specific LYN substrate 1) |
| | | | (LckBP1) (p75) |
| RAB21 KIAA0118 | Q9UL25 | RAB21_HUMAN | Ras-related protein Rab-21 |
| LSP1 WP34 | P33241 | LSP1_HUMAN | Lymphocyte-specific protein 1 (47 kDa actin- |
| | | | binding protein) (52 kDa phosphoprotein) |
| | | | (pp52) (Lymphocyte-specific antigen WP34) |
| CD44 LHR MDU2 | P16070 | CD44_HUMAN | CD44 antigen (CDw44) (Epican) |
| MDU3 MIC4 | | | (Extracellular matrix receptor III) (ECMR-III) |
| | | | (GP90 lymphocyte homing/adhesion |
| | | | receptor) (HUTCH-I) (Heparan sulfate |
| | | | proteoglycan) (Hermes antigen) (Hyaluronate |
| | | | receptor) (Phagocytic glycoprotein 1) (PGP-1) |
| | | | (Phagocytic glycoprotein I) (PGP-I) (CD |
| | | | antigen CD44) |
| HSPE1 | P61604 | CH10_HUMAN | 10 kDa heat shock protein, mitochondrial |
| | | | (Hsp10) (10 kDa chaperonin) (Chaperonin |
| | | | 10) (CPN10) (Early-pregnancy factor) (EPF) |
| MMP8 CLG1 | P22894 | MMP8_HUMAN | Neutrophil collagenase (EC 3.4.24.34) (Matrix |
| | | | metalloproteinase-8) (MMP-8) (PMNL |
| | | | collagenase) (PMNL-CL) |
| PIGR | P01833 | PIGR_HUMAN | Polymeric immunoglobulin receptor (PIgR) |
| | | | (Poly-Ig receptor) (Hepatocellular carcinoma- |
| | | | associated protein TB6) [Cleaved into: |
| | | | Secretory component] |
| ATP6V1G1 | O75348 | VATG1_HUMAN | V-type proton ATPase subunit G 1 (V- |
| ATP6G ATP6G1 | | | ATPase subunit G 1) (V-ATPase 13 kDa |
| ATP6J | | | subunit 1) (Vacuolar proton pump subunit G |
| | | | 1) (Vacuolar proton pump subunit M16) |
| ITGB2 CD18 | P05107 | ITB2_HUMAN | Integrin beta-2 (Cell surface adhesion |
| MFI7 | | | glycoproteins LFA-1/CR3/p150, 95 subunit |
| | | | beta) (Complement receptor C3 subunit beta) |
| | | | (CD antigen CD18) |
| LDHA PIG19 | P00338 | LDHA_HUMAN | L-lactate dehydrogenase A chain (LDH-A) |
| | | | (EC 1.1.1.27) (Cell proliferation-inducing |
| | | | gene 19 protein) (LDH muscle subunit) (LDH- |
| | | | M) (Renal carcinoma antigen NY-REN-59) |
| CHAMP1 | Q96JM3 | CHAP1_HUMAN | Chromosome alignment-maintaining |
| C13orf8 CAMP | | | phosphoprotein 1 (Zinc finger protein 828) |
| CHAMP | | | |
| KIAA1802 | | | |
| ZNF828 | | | |
| LMNB1 LMN2 | P20700 | LMNB1_HUMAN | Lamin-B1 |
| LMNB | | | |
| STMN1 C1orf215 | P16949 | STMN1_HUMAN | Stathmin (Leukemia-associated |
| LAP18 OP18 | | | phosphoprotein p18) (Metablastin) |
| | | | (Oncoprotein 18) (Op18) (Phosphoprotein |
| | | | p19) (pp19) (Prosolin) (Protein Pr22) (pp17) |
| NAMPT PBEF | P43490 | NAMPT_HUMAN | Nicotinamide phosphoribosyltransferase |
| PBEF1 | | | (NAmPRTase) (Nampt) (EC 2.4.2.12) (Pre-B- |
| | | | cell colony-enhancing factor 1) (Pre-B cell- |
| | | | enhancing factor) (Visfatin) |

Indeed, herewith disclosed are also the use of kits comprising the means for detecting and/or determining the expression of the one or more of the above-listed proteins in the indicated sample, as tools for the diagnosis and/or for the prognosis of EC.

Finally, another aspect of the invention is a computer-implemented method for carrying out the in vitro method as defined in the first aspect, in which after the determination of the level of expression of MDK, and optionally of one or more of the proteins for the diagnosis and/or for the prognosis of endometrial cancer, said level(s) are given a value and/or a score, and optionally are computed in a mathematical formula to obtain a computed value; wherein in function of the said level(s), score(s) and/or computed value(s), a decision is taken between the options of suffering or not from EC and/or between the options of suffering among different EC presenting different prognosis, including different histological subtypes and grades and different molecular features. In other words, a decision is taken between the options of suffering or not from EC and/or between the options of suffering among different EC subtypes, and/or between the options of suffering among different EC grades.

This aspect results from the algorithm for carrying out any of the methods as defined in this description. In the sense of the invention, the term “algorithm” is also synonymous of pannel or decision diagrams, predictors and combinatory of data to correctly categorize an individual sample.

Herewith disclosed is also a method for the diagnosis and/or for the prognosis of EC, the method comprising determining, in a sample from the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, a uterine fluid, uterine washings and combinations thereof, the presence and/or the level of expression of one or more of the following proteins: Midkine (MDK), Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A) and Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Agrin (AGR), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1 (LGALS1), ATPase H+ Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), and Exportin-2 (XPO2). In some examples, the method further comprises the step of determining one or more clinical or featuring parameters of the subject, in particular selected from the group consisting of blood pressure, glycemia, age, scores for grading or staging (i.e., grading/staging system of the Federation of Gynecology and Obstetrics (FIGO)), thickness of the endometrium, CA125 and HE4 molecular markers, AVB, and combinations thereof.

It is also disclosed a method for the diagnosis and/or for the prognosis of EC, the method comprising determining, in a sample from the female genital tract part including one or more of the vulvae, vagina, cervix, uterus, fallopian tubes, and ovaries and selected from a gynecologic sampling, including a cervical fluid, a cytology, a pap-smear like sample, an endometrial biopsy, an uterine fluid, uterine washings and combinations thereof, the presence and/or the level of expression of one or more of the proteins from the group listed in Table 1.

Also disclosed herewith is a particular prognosis method for the detection of histological type of EC, which comprises determining in a sample from the female genital tract part including one or more of the vulvae, vagina, cervix, uterus, fallopian tubes, and ovaries and selected from a gynecologic sampling, including a cervical fluid, a cytology, a pap-smear sample, a pap-smear like sample, an endometrial biopsy, an uterine fluid, uterine washings and combinations thereof, the presence and/or the level of expression of one or more of PIGR, PKM, optionally in combination with MDK or any other of the proteins in Table 1. In particular, the combination of PIGR with MDK provides AUC values near 0.846 for the correct determination (i.e., diagnosis) of the EC hystology. Of note also the combination of PIGR; RAB2; and MDK which gave an AUC value of 0.89.

Also disclosed herewith is a particular prognosis method for the grading of EC, which comprises determining in a sample from the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, an uterine fluid, uterine washings and combinations thereof, the presence and/or the level of expression of one or more of PIGR, HSPE1, optionally in combination with MDK or any other of the proteins in Table 1. In particular, the combination of PIGR with MDK provides AUC values near 0.920 for the correct determination (i.e., diagnosis) of the EC grade.

An additional aspect related with the diagnosis of EC, the invention provides a method for identifying a subject suspicious of suffering from EC, the method comprising:

a) determining, in vitro, the presence and/or the level of expression of midkine (MDK) in a sample from the female genital tract part including one or more of the vulvae, vagina, cervix, uterus, fallopian tubes, and ovaries and selected from a gynecologic sampling, including a cervical fluid, a cytology, a pap-smear sample, a pap-smear like sample, an endometrial biopsy, a uterine fluid, uterine washings and combinations thereof; and b) comparing the level of MDK of step (a) with a corresponding reference value or reference interval for each protein, said reference value or interval selected from a value or interval of values from a subject suffering from endometrial cancer, and/or comparing with a cut-off value discriminating among endometrial cancer and other gynecological disorders or conditions, and wherein the subject is diagnosed of endometrial cancer if the level of MDK is within the value or interval of values from a subject suffering from this cancer, and/or if the level of MDK in relation to the cut-off value is classified to the endometrial cancer group.

In a further aspect, the present invention provides a method of deciding or recommending whether to initiate a medical regimen of a subject suspicious of suffering endometrial carcinoma, which method comprises the steps of:
a) determining, in vitro, the presence and/or the level of expression of midkine (MDK) in a sample from the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, a uterine fluid, uterine washings and combinations thereof; and
b) comparing the level of MDK of step (a) with a corresponding reference value or reference interval for each protein, said reference value or interval selected from a value or interval of values from a subject suffering from endometrial cancer, and/or comparing with a cut-off value discriminating among endometrial cancer and other gynecological disorders or conditions, and wherein the subject is diagnosed of endometrial cancer if the level of MDK is within the value or interval of values from a subject suffering from this cancer, and/or if the level of MDK in relation to the cut-off value is classified to the endometrial cancer group.
wherein:
   i) if the subject is diagnosed of suffering from endometrial carcinoma, or of being suspicious of suffering from endometrial carcinoma, then the initiation of the medical regimen is recommended, and
   ii) if the patient is diagnosed of not suffering from endometrial carcinoma, the follow-up is performed optionally in consideration of the result of an examination of the patient by a physician.

By determining the marker level in a test sample, the skilled person can establish, additionally, which is the most suitable treatment that can be recommended, because the level detected in the sample may reflect the extension (i.e., severity) of the disease.

### BRIEF DESCRIPTION OF THE DRAWINGS

FIG. 1 shows the results obtained using an ELISA assay for determining MDK, tested in samples of a cohort of subjects and comparing EC vs non-EC patients (cervical sample, also called cervical fluid OR pap-smear like sample). In (A) Dotplot of the distribution of protein concentration between EC and non-EC patients. In (B) the a curve of an ROC analysis (Receiver Operating Characteristic analysis) of MDK assessed by ELISA.

FIG. 2 shows the results obtained using (A) mass spectrometry (LC-MS/MS PRM) or (B) an ELISA assay for determining MDK, tested in samples of a cohort of subjects and comparing EC vs non-EC patients. Dotplots illustrate the distribution of protein concentration between EC and non-EC patients. Also a ROC analysis (Receiver Operating Characteristic analysis) of MDK assessed by either LC-MS/MS or ELISA is depicted. In (C) correlation between the measurements of both approaches.

FIG. 3 shows the results obtained using an ELISA assay for determining MDK in uterine aspirates/pipelle biopsies/uterine fluids, tested in samples of a cohort of subjects and comparing EC vs non-EC patients. In (A) Dotplot of the distribution of protein concentration between EC and non-EC patients. In (B) the a curve of an ROC analysis (Receiver Operating Characteristic analysis) of MDK assessed by ELISA.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The present invention provides new biomarkers for the diagnosis and prognosis of endometrial cancer in a sample of the female genital tract (i.e., including one or more of the vulvae, vagina, cervix, uterus, fallopian tubes, and ovaries), said sample selected from a gynecologic sampling, including a cervical fluid, a cytology, a pap-smear sample, a pap-smear like sample containing fluids, an endometrial biopsy, a uterine fluid, uterine washings and combinations thereof.

The term “diagnosis” is known to the person skilled in the art. As used herein “diagnosis” is understood as becoming aware of a particular medical condition complication or risk in a subject; the determination of the nature of the disease or condition; or the distinguishing of one disease or condition from another. It refers both to the process of attempting to determine or identify the possible disease or disorder, and to the opinion reached by this process. A diagnosis, in the sense of diagnostic procedure, can be regarded as an attempt at classification of an individual's condition into separate and distinct categories that allow medical decisions about treatment and prognosis to be made. Subsequently, a diagnostic opinion is often described in terms of a disease or other condition. However, a diagnosis can take many forms. It might be a matter of detecting the presence and naming the disease, lesion, dysfunction or disability. It might be an exercise to attribute a category for management or for prognosis. It may indicate either degree of abnormality on a continuum or kind of abnormality in a classification. Thus, the term “diagnosis” also encompasses the “screening” or “differential diagnosis” of the subjects in order to classify the same in several differentiated groups including, in particular, asymptomatic subjects, a subject with risk of suffering from EC, a subject already diagnosed of suffering EC, the classification of subjects suffering from EC and presenting different prognosis, including different histological subtypes (in particular EEC vs NEEC) and grades and different molecular features, etc. The methods of the invention are, therefore, powerful screening tools for the correct classification of all the analyzed samples from the subjects.

In general terms, diagnostic markers listed in this description are those protein differentially detected at level expression in isolated samples of controls (non-cancer individuals) versus endometrial cancer samples (including characterization of the tumor, and/or several types of EC).

The term “patient” (or subject), as used herein, refers to any subject which show one or more signs or symptoms typically associated with EC. The term “patient”, as used herein, refers also to all animals classified as female mammals and includes, but is not restricted to, domestic and farm female animals, primates and humans. Preferably, the patient is a female human of any age or race.

The in vitro method of diagnosis, including screening, of the first aspect of the invention can be performed with a sample of: (a) an asymptomatic subject, (b) a subject which has already been identified as being suspicious of suffering from endometrial cancer, (c) a subject already diagnosed of endometrial cancer, as complementary confirmation diagnostic assay or (d) a subject with high risk of suffering the disease.

The term “reference value”, as used herein, relates to a predetermined criterion used as a reference for evaluating the values or data obtained from the samples collected from a subject. The reference value or reference level can be an absolute value (i.e., a cut-off value or cut-off discriminating value); a relative value; a value that has an upper or a lower limit; a range of values (i.e., a range of possible cut-off values); an average value; a median value, a mean value, or a value as compared to a particular control or baseline value. A reference value or reference range can be based on an individual sample value, such as for example, a value obtained from a sample from the subject being tested, but at an earlier point in time. The reference value or range can be based on a large number of samples, such as from population of subjects of the chronological age matched group, or based on a pool of samples including or excluding the sample to be tested. Reference values have been determined for the biomarkers of the invention. The reference value for a protein (i.e., MDK), may be from a lower and an upper value as will be disclosed in view of examples below. Range of values of each biomarker (protein levels) and particular combinations of the values of the different biomarkers provide for correct classification of subjects with high sensitivity and specificity.

If the level of expression is determined at the protein level, then the “reference expression level” is a predefined value of protein quantity, whereas if the level of expression is determined at the mRNA level, then the “reference expression level” is a predefined value of mRNA quantity. The samples are taken from a subject or group of subjects wherein the presence, absence, stage, histological subtype or grade, or course of the disease has been properly performed previously. This value is used as a threshold to discriminate subjects wherein the condition to be analyzed is present from those wherein such condition is absent (i.e., subject having endometrial cancer from subjects free of endometrial cancer), to determine the histological subtype of the disease, the risk of developing or of being suffering from endometrial carcinoma, among others. This reference control level is also useful for determining whether the subject has to initiate a medical regimen and how effective the regimen is. The subject or subjects from whom the “reference control level” is derived may include subject/s wherein the condition is absent, subject/s wherein the condition is present, or both. The skilled person in the art, making use of the general knowledge, is able to choose the subject or group of subjects more adequate for obtaining the reference control level for each of the methods of the present invention. Methods for obtaining the reference value from the group of subjects selected are well-known in the state of the art (Burtis C. A. et al., 2008, Chapter 14, section “Statistical Treatment of Reference Values”). In a particular case “reference control level” is a cut-off value defined by means of a conventional ROC analysis (Receiver Operating Characteristic analysis). As the skilled person will appreciate, optimal cut-off value will be defined according to the particular applications of the diagnostic or prognostic method: purpose, target population for the diagnosis or prognosis, balance between specificity and sensibility, etc.

“Prognosis” as used herein refers to the prediction of the probable progression and outcome of a disease. It includes: neoplasm grading (attempt to express in replicable terms the level of cell differentiation in neoplasms as increasing anaplasia correlates with the aggressiveness of the neoplasm), neoplasm staging (attempt to express in replicable terms the extent of the neoplasm in the patient), neoplasm histological subtype, and neoplasm molecular subtype. As used herein prognosis means, in particular embodiments, differentiation between endometriod endometrial cancer and non-endometriod endometrial cancers, or differentiation between low and high histological grades of endometrial cancers, or differentiation between molecular subytpes of endometrial cancers, or differentiation between patients at high or low risk of recurrence.

As previously indicated, the first aspect of the invention is a method of diagnosis and/or for the prognosis of endometrial cancer, the method comprising determining the presence and/or the level of expression of MDK in an isolated sample from the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, a uterine fluid, uterine washings and combinations thereof.

The sample from the female genital tract from the vulvae to the cervix is a sample that results from the common and routine inspection performed usually once a year by gynecologists.

In a particular embodiment of the first aspect, the sample is a pap-smear like sample, and specifically the fluid contained in a pap-smear like and/or cervical fluid. In other words, the sample is a cervical sample and specifically, the fluid contained in a cervical sample. This cervix fluid is easily obtainable using cytobrushs, in the same way they are used for the cytology analysis of the routine gynecological inspection.

In another particular embodiment, the sample is a uterine aspirate (also called pipelle biopsy or uterine fluid). This is commonly known as the sample that results from the common and routine gynecological inspection used for determining the existence of carcinomas in the gynecological tract and which is less damaging and discomforting than a biopsy by histeroscopy or dilatation&curettage. It is of relevance that MDK allows distinguishing between EC and non-EC also in this kind of sample.

This is the first time in this kind of routine sample MDK is detected as informative. Thus, the invention relates to the use of MDK as diagnostic marker in a sample from the female genital tract part including one or more of the vulvae, vagina, cervix, uterus, fallopian tubes, and ovaries and selected from a gynecologic sampling, including a cervical fluid, a cytology, a pap-smear like sample, a pap-smear sample, an endometrial biopsy, a uterine fluid, uterine washings and combinations thereof. In particular it relates to the use of MDK as diagnostic and/or prognostic marker of EC.

As will be illustrated in the examples section, when MDK is determined and at certain levels of expression in any sample of this part of the female genital tract, high sensitivities and specificities are achieved (e.g., AUC=0.910, SE=82.8, SP=87.8). When the level of expression of additional markers were also determined in the sample isolated from these structures of the genital tract (i.e., in particular a fluid from the cervix), the specificity was maintained while the sensitivity increased, which allowed the classification of most of the true positive patients.

Thus, in another particular embodiment of the method of the first aspect, it further comprises determining the presence and/or the level of expression of one or more of the other proteins of Table 1. In another more particular embodiment, determining the presence and/or the level of expression of one or more of the following proteins:

Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A) and Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Agrin (AGR), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1 (LGALS1), ATPase H+Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), and Exportin-2 (XPO2).

In a more particular embodiment, the method comprises determining the presence and/or the level of expression of two, three, four, five, six, seven, eight, nine, and ten of the proteins always including MDK in the panel of proteins. In yet another particular embodiment of the in vitro method, it comprises determining the presence and/or the level of expression of two, three, or four of the proteins, including MDK in the panel of proteins.

In a more particular embodiment of the method, it comprises determining in the isolated sample the presence and/or the level of expression of the proteins in at least one binary set of the group listed in any one of Tables 4 and 6.

In a more particular embodiment of the method, it comprises determining in the isolated sample the presence and/or the level of expression of the proteins in at least one binary set of the group consisting of: MDK, LCN2; MDK, FN1; MDK, PLBD1; MDK, APOB; MDK, CD44; MDK, FCGBP; MDK, EPS8L1; MDK, ANXA3; MDK, C4BPA; MDK, MMP8; MDK, NEDD-8; MDK, CAMP; MDK, TIMP2; MDK, HSPE1; MDK, CALU; MDK, C1QA; MDK, LDHA; MDK, TIMP2; MDK, PIGR; MDK, KRT8; MDK, PPL; MDK, SPRR1A; MDK, STMN1; MDK, CAPS; MDK, CA1; MDK, CCT6A; MDK, VIM; MDK, TCP1; and MDK, AGRN.

In a more particular embodiment of the method, it comprises determining in the isolated sample the presence and/or the level of expression of the proteins in at least one ternary set of the group listed in any one of Tables 5 and 7.

In another particular embodiment, the method comprises determining in the isolated sample the presence and/or the level of expression of the proteins in at least one ternary set of the group consisting of: MDK, ANXA7, CSE1L; MDK, LCN2, FGFBP1; MDK, LCN2, ANXA7; MDK, FGFBP1, LCN2; MDK, APOF, FCGBP; MDK, IMPA1, FCGBP; MDK, LCN2, CD44; MDK, STX7, FCGBP; MDK, ITIH2, FCGBP; MDK, LCN2, EPS8L1; MDK, FGFBP, EPS8L1; MDK, ANXA7, FCGBP; MDK, LCN2, PLBD1; MDK, FCGBP, CD44; MDK, PLBD1, FCGBP; MDK, ANXA3, FCGBP; MDK, PLBD1, LGALS1; MDK, LCN2, IMPA1; MDK, FCGBP, VIM; MDK, FCGBP, LMNB1; MDK, PLBD1, ATP6V1G1; MDK, APOB, PKM; MDK, ITIH2, LCN2; MDK, PLBD1, CALU; MDK, LCN2, APOF; MDK, PLBD1, EPS8L1; MDK, APOC1, ANXA7; MDK, FCGBP, GYG1; MDK, PLBD1, PKM; MDK, FCGBP, LSP1; MDK, TIMP2, CD44; MDK, ANXA3, PLBD1; MDK, PLBD1, HSPE1; MDK, PLBD1, CA1; MDK, FCGBP, HCLS1; MDK, STX7, IMPA1; MDK, ANXA7, STX7; MDK, STX7, EPS8L1; MDK, PLBD1, LSP1; MDK, C1R, ANXA7; MDK, STX7, PEA15; MDK, PLBD1, HCLS1; MDK, LCAT, FCGBP; MDK, PLBD1, LDHA; MDK, APOB, S100A9; MDK, ANXA7, SCEL; MDK, SERPINA3, FCGBP; MDK, FCGBP, ITGB2; MDK, SCEL, ANXA3; MDK, S100A9, PLBD1; MDK, S100A9, LSP1; MDK, C1R, FCGBP; MDK, FGFBP1, FCGBP; MDK, APOC1, S100A9; MDK, PPL, PLBD1; MDK, TCP1, PEA15; MDK, ANXA3, LSP1; MDK, PPL, IMPA1; MDK, S100A9, MMP8; MDK, FGFBP1, PPL; and MDK, S100A9, PPL.

In a more particular embodiment, the method comprises determining in the isolated sample the presence and/or the level of expression of the set defined by MDK, ANXAand CSE1L.

In yet another particular embodiment, the method comprises determining in the isolated sample the presence and/or the level of expression of the proteins in at least one quaternary set of the group consisting of: MDK, LCN2, PEA15, LDHA; MDK, LCN2, PEA15, PKM; MDK, FGFBP1, FCGBP, EPS8L1; MDK, LCN2, HSPE1, CD44; MDK, FGFBP1, EPS8L1, CTNNB1; MDK, LCN2, IMPA1, LDHA; MDK, IMPA1, FCGBP, PEA15; MDK, IMPA1, FCGBP, PKM; MDK, FCGBP, IMPA1, PEA15; MDK, CCT6A, FGFBP1, EPS8L1; MDK, STX7, FCGBP, PKM; MDK, IMPA1, FCGBP, GYG1; MDK, FGFBP1, SCEL, EPS8L1; MDK, STX7, FCGBP, CA1; MDK, STX7, FCGBP, NAMPT; MDK, FGFBP1, EPS8L1, CAPS; MDK, STX7, FCGBP, FCGBP; MDK, SCEL, STX7, FCGBP; MDK, FGFBP1, ANXA7, CAPS; MDK, FGFBP1, EPS8L1, RAB21; MDK, SCEL, STX7, FCGBP; MDK, STX7, FCGBP, NEDD8-MDP1; MDK, ITIH2, FCGBP, CAPS; MDK, FGFBP1, EPS8L1, PKM; MDK, IMPA1, FCGBP, HCLS1; MDK, APOF, ANXA3, FCGBP; MDK, FGFBP1, EPS8L1, STMN1; MDK, STX7, FCGBP, LGALS1; MDK, FCGBP, LSP1, VIM; MDK, FGFBP1, EPS8L1, LGALS1; MDK, ANXA3, FCGBP, LMNB1; MDK, FCGBP, IMPA1, MMP8; MDK, FGFBP1, EPS8L1, STMN1; MDK, SCEL, IMPA1, PEA15; MDK, FGFBP1, ANXA7, LGALS1; MDK, TCP1, PEA15, LMNB1; MDK, FCGBP, GYG1, CA1; MDK, SCEL, IMPA1, PKM; MDK, FGFBP1, EPS8L1, LDHA; MDK, FCGBP, KRT8, GYG1; MDK, FCGBP, GYG1, LMNB1; MDK, FCGBP, GYG1, LSP1; MDK, FCGBP, KRT8, GYG1; MDK, FCGBP, GYG1, LGALS1; MDK, FCGBP, CAPS, GYG1; MDK, FGFBP1, EPS8L1, LDHA; MDK, FCGBP, CAPS, GYG1; MDK, C1R, FCGBP, LGALS1; MDK, FGFBP1, SCEL, PEA15; MDK, FGFBP1, TIMP2, CHMP4B; MDK, ANXA3, STMN1, PKM; MDK, ANXA7, SCEL, CHMP4B; MDK, FCGBP, GYG1, LDHA; MDK, S100A9, MMP8, LGALS1; MDK, C1R, FCGBP, GYG1; MDK, C4BPA, FGFBP1, PPL; MDK, ITIH2, FGFBP1, PPL; and MDK, APOC1, FGFBP1, PPL.

In another embodiment of the first aspect, optionally in combination with any of the embodiments above or below, the method comprises the following steps:
a) determining, in vitro, the level of expression of MDK, optionally in combination with one or more of the proteins listed in any one of previous embodiments, in the isolated sample from the genital tract of the female;
b) comparing the level of MDK and, if determined, of the one or more other the proteins of step (a) with a corresponding reference value or reference interval for each protein, said reference value or interval selected from a value or interval of values from a subject suffering from endometrial cancer, and/or comparing with a cut-off value discriminating among endometrial cancer and other endometriod disorders or conditions of the endometrium; and
c) wherein the subject is diagnosed of endometrial cancer if at least the level of MDK is within the value or interval of values from a subject suffering from this cancer, and/or if at least the level of MDK in relation to the cut-off value is classified to the endometrial cancer group.

The expression other gynecological disorders or conditions include, without limitation, a healthy endometrium, endometriosis, endometrial polyps, endometrial myomas, atrophic endometrium, physiological damage of the uterus, or any other condition that can cause abnormal vaginal bleeding.

In another particular embodiment of the methods disclosed in the invention, it further comprises the step of determining one or more clinical or featuring parameters of the subject, in particular selected from the group consisting of blood pressure, glycemia, age, scores for grading or staging (i.e., stages of the Federation of Gynecology and Obstetrics (FIGO)), thickness of the endometrium, CA125 and HE4 molecular markers, AVB, and combinations thereof.

Another particular embodiment of the method according to the first aspect or any of the embodiments thereof, the level of expression of MDK and of other proteins is determined at the protein level.

In a more particular embodiment, the protein level is determined by an assay or technology selected from the group consisting of an immunoassay, a bioluminescence assay, a fluorescence assay, a chemiluminescence assay, electrochemistry assay, a lateral flow assay, mass spectrometry, and combinations thereof.

In another particular embodiment, the level of expression of protein is determined using an antibody or a fragment thereof able to specifically bind to the protein.

More in particular, said antibody or fragment thereof forms part of a kit.

Alternatively, the level of expression is determined at the mRNA level. Below is further explained in more detail this alternative method.

In a second aspect, the invention relates to the use of MDK, as in vitro marker for the diagnosis and/or for the prognosis of endometrial cancer in a sample from the female genital tract part including one or more of the vulvae, vagina, cervix, uterus, fallopian tubes, and ovaries and selected from a gynecologic sampling, including a cervical fluid, a cytology, a pap-smear sample, a pap-smear like sample, an endometrial biopsy, a uterine fluid, uterine washings and combinations thereof.

In a particular embodiment of the second aspect, the sample is a pap-smear, and specifically the fluid contained in a pap-smear and/or cervical fluid.

In also another particular embodiment of the second aspect, MDK is used as in vitro marker in a panel of multiple biomarkers comprising one or more of Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A) and Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Agrin (AGR), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1(LGALS1), ATPase H+Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), and Exportin-2 (XPO2).

In also another particular embodiment of the second aspect, MDK is used as in vitro marker in a panel of multiple biomarkers comprising one or more of the other proteins from the group listed in Table 1.

These pannels of biomarkers including MDK comprise, in a particular embodiment from two to all the listed markers. In particular, the pannels comprise two, three, four, five, six, seven, eight, nine, and ten biomarkers, being one of them MDK.

Disclosed are also pannels of biomarkers comprising one or more of the proteins of Table 1. In a particular example, pannels including from two to all the listed markers. In particular, the pannels comprise two, three, four, five, six, seven, eight, nine, and ten biomarkers.

Diagnosis of EC with MDK and/or the one or more proteins listed above can be carried out, as indicated, by the use of kits comprising means for determining the level of expression of these proteins. As previously indicated, a third aspect of the invention is a kit comprising a solid support and means for detecting the presence and/or for determining the level of expression of MDK and optionally means for detecting the presence and/or for determining the level of expression of one or more proteins listed in Table 1, in particular one or more selected from the group consisting of Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A) and Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Agrin (AGR), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1(LGALS1), ATPase H+Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), and Exportin-2 (XPO2).

A “device” or “kit” in the sense of the invention is an assay or method to determine a (combination of) biomarker (levels of proteins of interest in a sample) or panel of biomarkers according to the invention that can be used to perform an assay or method for the diagnosis and/or prognosis of EC or for the selection of a patient for a medical regimen once diagnosed. Examples are carrier plates, test stripes, biochip arrays, electrochemical sensors, or the like known in the art including the reagent means to detecting the presence and level of the proteins of interest. The kits, as used herein, refer to a product containing the different reagents (or reagent means) necessary for carrying out the methods of the invention packed so as to allow their transport and storage. Materials suitable for packing the components of the kit include crystal, plastic (e.g. polyethylene, polypropylene, polycarbonate), bottles, vials, paper, or envelopes. Instructions in different formats for carrying out the method are, in some embodiments also included in the said kits. Particular formats of the instructions are selected from leaflets, electronic supports capable of storing instructions susceptible of being read or understood, such as, for example, electronic storage media (e.g. magnetic disks, tapes), or optical media (e.g. CD-ROM, DVD), or audio materials.

In a particular embodiment of the kit of the invention, it comprises means (i.e., reagent means) for detecting and/or quantifying the level of expression of at least one binary set of the group consisting of: MDK, LCN2; MDK, FN1; MDK, PLBD1; MDK, APOB; MDK, CD44; MDK, FCGBP; MDK, EPS8L1; MDK, ANXA3; MDK, C4BPA; MDK, MMP8; MDK, NEDD-8; MDK, CAMP; MDK, TIMP2; MDK, HSPE1; MDK, CALU; MDK, C1QA; MDK, LDHA; MDK, TIMP2; MDK, PIGR; MDK, KRT8; MDK, PPL; MDK, SPRR1A; MDK, STMN1; MDK, CAPS; MDK, CA1; MDK, CCT6A; MDK, VIM; MDK, TCP1; and MDK, AGRN.

In another particular embodiment, the binary set is selected from at least one of Tables 4 and/or 6 below (see in examples).

In another particular embodiment of the kit of the invention, it comprises means (i.e., reagent means) for detecting and/or quantifying the level of expression of at least one ternary set of the group consisting of: MDK, ANXA7, CSE1L; MDK, LCN2, FGFBP1; MDK, LCN2, ANXA7; MDK, FGFBP1, LCN2; MDK, APOF, FCGBP; MDK, IMPA1, FCGBP; MDK, LCN2, CD44; MDK, STX7, FCGBP; MDK, ITIH2, FCGBP; MDK, LCN2, EPS8L1; MDK, FGFBP, EPS8L1; MDK, ANXA7, FCGBP; MDK, LCN2, PLBD1; MDK, FCGBP, CD44; MDK, PLBD1, FCGBP; MDK, ANXA3, FCGBP; MDK, PLBD1, LGALS1; MDK, LCN2, IMPA1; MDK, FCGBP, VIM; MDK, FCGBP, LMNB1; MDK, PLBD1, ATP6V1G1; MDK, APOB, PKM; MDK, ITIH2, LCN2; MDK, PLBD1, CALU; MDK, LCN2, APOF; MDK, PLBD1, EPS8L1; MDK, APOC1, ANXA7; MDK, FCGBP, GYG1; MDK, PLBD1, PKM; MDK, FCGBP, LSP1; MDK, TIMP2, CD44; MDK, ANXA3, PLBD1; MDK, PLBD1, HSPE1; MDK, PLBD1, CA1; MDK, FCGBP, HCLS1; MDK, STX7, IMPA1; MDK, ANXA7, STX7; MDK, STX7, EPS8L1; MDK, PLBD1, LSP1; MDK, C1R, ANXA7; MDK, STX7, PEA15; MDK, PLBD1, HCLS1; MDK, LCAT, FCGBP; MDK, PLBD1, LDHA; MDK, APOB, S100A9; MDK, ANXA7, SCEL; MDK, SERPINA3, FCGBP; MDK, FCGBP, ITGB2; MDK, SCEL, ANXA3; MDK, S100A9, PLBD1; MDK, S100A9, LSP1; MDK, C1R, FCGBP; MDK, FGFBP1, FCGBP; MDK, APOC1, S100A9; MDK, PPL, PLBD1; MDK, TCP1, PEA15; MDK, ANXA3, LSP1; MDK, PPL, IMPA1; MDK, S100A9, MMP8; MDK, FGFBP1, PPL; and MDK, S100A9, PPL.

In another particular embodiment, the ternary set is selected from at least one of Tables 5 and/or 7 below (see in examples).

In another particular embodiment of the kit of the invention, it comprises means (i.e., reagent means) for detecting and/or quantifying the level of expression of at least one quaternary set of the group consisting of: MDK, LCN2, PEA15, LDHA; MDK, LCN2, PEA15, PKM; MDK, FGFBP1, FCGBP, EPS8L1; MDK, LCN2, HSPE1, CD44;. MDK, FGFBP1, EPS8L1, CTNNB1; MDK, LCN2, IMPA1, LDHA; MDK, IMPA1, FCGBP, PEA15; MDK, IMPA1, FCGBP, PKM; MDK, FCGBP, IMPA1, PEA15; MDK, CCT6A, FGFBP1, EPS8L1; MDK, STX7, FCGBP, PKM; MDK, IMPA1, FCGBP, GYG1; MDK, FGFBP1, SCEL, EPS8L1; MDK, STX7, FCGBP, CA1; MDK, STX7, FCGBP, NAMPT; MDK, FGFBP1, EPS8L1, CAPS; MDK, STX7, FCGBP, FCGBP; MDK, SCEL, STX7, FCGBP; MDK, FGFBP1, ANXA7, CAPS; MDK, FGFBP1, EPS8L1, RAB21; MDK, SCEL, STX7, FCGBP; MDK, STX7, FCGBP, NEDD8-MDP1; MDK, ITIH2, FCGBP, CAPS; MDK, FGFBP1, EPS8L1, PKM; MDK, IMPA1, FCGBP, HCLS1; MDK, APOF, ANXA3, FCGBP; MDK, FGFBP1, EPS8L1, STMN1; MDK, STX7, FCGBP, LGALS1; MDK, FCGBP, LSP1, VIM; MDK, FGFBP1, EPS8L1, LGALS1; MDK, ANXA3, FCGBP, LMNB1; MDK, FCGBP, IMPA1, MMP8; MDK, FGFBP1, EPS8L1, STMN1; MDK, SCEL, IMPA1, PEA15; MDK, FGFBP1, ANXA7, LGALS1; MDK, TCP1, PEA15, LMNB1; MDK, FCGBP, GYG1, CA1; MDK, SCEL, IMPA1, PKM; MDK, FGFBP1, EPS8L1, LDHA; MDK, FCGBP, KRT8, GYG1; MDK, FCGBP, GYG1, LMNB1; MDK, FCGBP, GYG1, LSP1; MDK, FCGBP, KRT8, GYG1; MDK, FCGBP, GYG1, LGALS1; MDK, FCGBP, CAPS, GYG1; MDK, FGFBP1, EPS8L1, LDHA; MDK, FCGBP, CAPS, GYG1; MDK, C1R, FCGBP, LGALS1; MDK, FGFBP1, SCEL, PEA15; MDK, FGFBP1, TIMP2, CHMP4B; MDK, ANXA3, STMN1, PKM; MDK, ANXA7, SCEL, CHMP4B; MDK, FCGBP, GYG1, LDHA; MDK, S100A9, MMP8, LGALS1; MDK, C1R, FCGBP, GYG1; MDK, C4BPA, FGFBP1, PPL; MDK, ITIH2, FGFBP1, PPL; and MDK, APOC1, FGFBP1, PPL.

In another particular embodiment of the first aspect, the method for the diagnosis and/or prognosis in which MDK levels are determined, in particular in a gynecological sample and/or cervical sample, is a method for the diagnosis of recurrence or risk of recurrence of endometrial cancer, which further comprises determining in the isolated sample of the female the presence and/or the level of expression of one or more of the proteins selected from the group consisting of: MUC1, PRSS8, PNP, APEH, MUC16, C9, SERPINC1, SERPINA1, F2, AMBP, HP, SERPINA3, CFB, ORM2, CAT, GNAI2, A1BG, FN1, C7. ASTRGL1, B4GALT1, CAPS, CBX3, CD163, CDV3, DMBT1, DSG3, EHD1, GOLM1, MUC5AC, NME1, NT5E, PDLIM5, RDX, and VASP.

MDK in combination with the one or more of the other listed in the previous paragraph are, in a particular embodiment determined in a cervical sample and/or in an uterine aspirate.

Thus, also disclosed is a method for the diagnosis of recurrence or risk of recurrence of endometrial cancer, in which one or more of the following proteins (i.e., the presence or absence and/or their levels) are determined in an isolated sample of the female genital tract part including one or more of the vulvae, vagina, cervix, uterus, fallopian tubes, and ovaries and selected from a gynecologic sampling, including a cervical fluid, a cytology, a pap-smear sample, a pap-smear like sample, an endometrial biopsy, a uterine fluid, uterine washings and combinations thereof: MUC1, PRSS8, PNP, APEH, MUC16, C9, SERPINC1, SERPINA1, F2, AMBP, HP, SERPINA3, CFB, ORM2, CAT, GNAI2, A1BG, FN1, C7. ASTRGL1, B4GALT1, CAPS, CBX3, CD163, CDV3, DMBT1, DSG3, EHD1, GOLM1, MUC5AC, NME1, NT5E, PDLIM5, RDX, and VASP.

As will be illustrated in the examples below, these markers gave a noteworthy accuracy (AUC>0.75) to differentiate between recurrent and non-recurrent EC patients. In particular, the one or more markers selected from ASTRGL1, B4GALT1, CAPS, CBX3, CD163, CDV3, DMBT1, DSG3, EHD1, GOLM1, MUC16, MUC5AC, NME1, NT5E, PDLIM5, PRSS8, RDX, and VASP, allowed such a differentiation with only the presence-absence statistical analysis, in the isolated sample.

All the particular embodiments regarding, for example, the type of sample, or number of markers that are determined indicated for the method of the first aspect do also apply to either the method that allows the diagnosis of recurrence or risk of recurrence of endometrial cancer.

For “recurrence”, also known as relapse or recidivism is a recurrence of a past (typically medical) condition. The “risk of recurrence” relates to the probability of such a recurrence.

Thus, in a particular embodiment of method for the diagnosis of recurrence or risk of recurrence of endometrial cancer, the method comprises the following steps:
a) determining, in vitro, the level of expression of one or more of the listed proteins, in the isolated sample from the genital tract of the female;
b) comparing the level of these one or more other proteins of step (a) with a corresponding reference value or reference interval for each protein, said reference value or interval selected from a value or interval of values from a subject suffering from endometrial cancer resulting from a recidivism; and
c) wherein the subject is diagnosed of recurrence of endometrial cancer if at least the level of one of the proteins is within the value or interval of values from a subject suffering from a recurrence of this cancer.

In another particular embodiment of the first aspect, the method for the diagnosis and/or prognosis in which MDK levels are determined, in particular in a gynecological sample and/or cervical sample, is a method for the diagnosis of endometrial cancer subtype and/or endometrial cancer molecular classification, and which further comprises determining in the isolated sample of the female the presence and/or the level of expression of one or more of the proteins selected from the group consisting of: LBP, VWF, GPLD1, SAA4, APOF, C4BPA, SPRR1A, SERPIND1, APOB, SCEL, LCAT, SERPINA3, LMO7, C1R, MUC4, FN1, SPRR1B, C1QA, ITIH2, TIMP2, APOC1, GRN, ANXA3, S100A9, PLBD1, PIGR, SERPINH1, HSPE1.

MDK in combination with the one or more of the other listed in the previous paragraph are, in a particular embodiment determined in a cervical sample and/or in an uterine aspirate.

Thus, also disclosed herewith is a method for the diagnosis of endometrial endometrial cancer subtype and/or endometrial cancer molecular classification, in which one or more of the following proteins (i.e., the presence or absence and/or their levels) are determined in an isolated sample of the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, a uterine fluid, uterine washings and combinations thereof: LBP, VWF, GPLD1, SAA4, APOF, C4BPA, SPRR1A, SERPIND1, APOB, SCEL, LCAT, SERPINA3, LMO7, C1R, MUC4, FN1, SPRR1B, C1QA, ITIH2, TIMP2, APOC1, GRN, ANXA3, S100A9, PLBD1, PIGR, SERPINH1, HSPE1.

All the particular embodiments regarding, for example, the type of sample, or number of markers that are determined indicated for the method of the first aspect do also apply to the method that allows the diagnosis of type of endometrial cancer in terms of molecular distinction between them.

In a particular example of the method for the diagnosis of endometrial cancer molecular subtype, the subtypes are selected from the group consisting of POLE ultramutated or POLE mutated (POLEmut), MSI: microsatellite instable (MSI) or MMR deficient, based on loss of mismatch repair protein expression (MMRd); Copy Number Low (CN-Low) or no specific molecular profile (NSMP); Copy Number High (CN-High) or p53 abnormal, based on mutant-like immunostaining (p53abn).

Data in examples illustrate the statistical significance between EC subtypes obtained with each of these markers analysed in a pap-smear like sample of patients (also called cervical sample, cervical fluid).

In a particular embodiment of method for the diagnosis of for the diagnosis of endometrial cancer subtype, the method comprises the following steps:
a) determining, in vitro, the level of expression of one or more of the listed proteins, in the isolated sample from the genital tract of the female;
b) comparing the level of these one or more other proteins of step (a) with a corresponding reference value or reference interval for each protein, said reference value or interval selected from a value or interval of values from a subject suffering from a particular endometrial cancer subtype; and
c) wherein the subject is diagnosed of said endometrial cancer subtype if at least the level of one of the proteins is within the value or interval of values from a subject suffering from that endometrial cancer subtype.

In another particular embodiment of the kits of the invention, the means for detecting the level of expression of the proteins are means for carrying out an assay or technology selected from the group consisting of an immunoassay, a bioluminescence assay, a fluorescence assay, a chemiluminescence assay, an electrochemistry assay, a lateral flow assay, mass spectrometry, and combinations thereof.

In even a more particular embodiment, the means for detecting the level of expression of the proteins in the kits are antibodies or fragments thereof.

In yet another more particular embodiment, the kits are for carrying out an enzyme-linked immunosorbent assay (ELISA).

In another particular embodiment of the kits of the invention, they further comprise a panel diagram, to categorize an individual sample.

In a preferred embodiment, the reagent means for assaying the levels of the different biomarkers (proteins) comprise at least 10%, at least 20%, at least 30%, at least 40%, at least 50%, at least 60%, at least 70%, at least 80%, at least 90% or at least 100% of the total amount of reagents for assaying biomarkers forming the kit. Thus, in the particular case of kits comprising reagents for assaying the levels of MDK (and optionally of one or more of the previous listed proteins), the reagents specific for said biomarkers (i.e.

antibodies which bind specifically to the proteins) comprise at least 10%, at least 20%, at least 30%, at least 40%, at least 50%, at least 60%, at least 70%, at least 80%, at least 90% or at least 100% of the antibodies present in the kit. These kits are, thus, simplified kits including mainly the reagent means for detecting the levels of the MDK (and optionally of one or more of the previous listed proteins).

In another particular embodiment, the kits of the invention are conceived as point of care tests. More in particular they are in form of lateral flow tests or an electrochemical sensor.

In another particular embodiment the kit according to the invention comprises a support (in particular a solid support) and one or more sample inlet ports for deposition of a biofluid sample, in particular whole fluid of the cervix; a reaction area comprising the means/reagents that bind specifically to the marker proteins, in particular antibodies; and wherein the sample inlet port is connected with the reaction area. In another more particular embodiment, the kit comprises as many sample inlet ports as markers (one, two or three or four) to be detected and corresponding reaction areas connected thereto. In another embodiment the kit comprises one single inlet import and as capillary tracks connecting to as many reactive areas, said capillary tracks conducting part of the sample to each corresponding connected reaction area. The kits comprising more than one reaction areas are multiplex kits.

In a more particular embodiment, the kits comprise means for detecting the level of expression of a combination from 2 to 80 sets.

In the particular case when the kits of the invention are ELISA kits, they comprise a solid support and antibodies or fragments thereof which specifically bind to the target proteins to be detected, these antibodies being conjugated with a reporter molecule capable of producing a signal.

The “solid support” includes a nitrocellulose membrane, glass or a polymer. The most commonly used polymers being cellulose, polyacrylamide, nylon, polystyrene, polyvinyl chloride or polypropylene. The solid supports may be in the form of strips, tubes, beads, discs or microplates, or any other surface suitable for conducting an immunoassay.

The “reporter molecule” as used in the present specification is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e., radioisotopes). In the case of an enzyme immunoassay, an enzyme is conjugated to the second antibody, generally by means of glutaraldehyde or periodate. As will be readily recognized, however, a wide variety of different conjugation techniques exist, which are readily available to those skilled in the art. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable colour change. For example, 5-bromo-4-chloro-3-indolyl phosphate/nitroblue tetrazolium is suitable for use with alkaline phosphatase conjugates; for peroxidase conjugates, 1,2-phenylenediamine, 5-aminosalicylic acid, 3,3:5,5:tetra methyl benzidine or tolidine are commonly used. It is also possible to employ fluorogenic substrates, which yield a fluorescent product rather than the chromogenic substrates noted above. Examples of fluorogenic substrates are fluorescein and rhodamine. When activated by illumination with light of a particular wave-length, the fluorochrome-labelled antibody absorbs the light energy, inducing a state of excitability in the molecule, followed by emission of the light at a characteristic colour visually detectable with a light microscope. Immunofluorescence and EIA techniques are both well established in the art and are particularly preferred for the present method. However, other reporter molecules, such as radioisotope, chemiluminescent, and bioluminescent molecules and/or dyes and other chromogenic substances, may also be employed.

The choice of a particular reporter molecule conjugated antibody will be, for the most part, determined by the intended use and user of the test kit of the present invention.

Binding assays for measuring biomarker levels may use solid phase or homogenous formats. Suitable assay methods include sandwich or competitive binding assays.

Examples of sandwich immunoassays are described in U.S. Pat. Nos. 4,168,146 and 4,366,241, both of which are incorporated herein by reference in their entireties. Examples of competitive immunoassays include those disclosed in U.S. Pat. Nos. 4,235,601, 4,442,204 and 5,208,535, each of which are incorporated herein by reference in their entireties.

Multiple biomarkers may be measured using a multiplexed assay format, e.g., multiplexing through the use of binding reagent arrays, multiplexing using spectral discrimination of labels, multiplexing of flow cytometric analysis of binding assays carried out on particles, e.g., using the Luminex® system.

The assays (methods and kits) of the present invention may be conducted by any suitable method. In one embodiment, biomarker levels are measured in a single sample, and those measurement may be conducted in a single assay chamber or assay device, including but not limited to a single well of an assay plate, a single assay cartridge, a single lateral flow device, a single assay tube, etc. Biomarker levels may be measured using any of a number of techniques available to the person of ordinary skill in the art, e.g., direct physical measurements (e.g., mass spectrometry) or binding assays (e.g., immunoassays, agglutination assays and immunochromatographic assays). The method may also comprise measuring a signal that results from a chemical reactions, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

In another aspect, the invention relates to the use of the kit of the invention according to the third aspect and/or any one of its embodiments, for the diagnosis and/or prognosis of EC.

Thus, in a particular embodiment, the invention relates to the use of the kit of the invention in any of the methods of the invention.

In any of the embodiments provided above or below, for any of the aspects and embodiments of the invention, the level of expression of MDK and optionally of any other protein is determined at the protein level. In this embodiment, the protein marker(s) include, but do not limit to, native-sequence peptides, isoforms, chimeric polypeptides, all homologs, fragments, and precursors of the markers, including modified forms of the polypeptides and derivatives thereof.

In particular embodiments provided above or below, the level of expression is determined by immunochemistry, as indicated. Next paragraphs illustrate in more detail this option.

The term “immunochemistry” as used herein refers to a variety of techniques for detecting antigens (usually proteins and peptides, and in the present case any of the proteins listed above alone or in combination) in a sample by exploiting the principle of antibodies binding specifically to said antigens. Visualizing an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as fluorescein or rhodamine. The immunochemistry technique can be direct or indirect. The direct method is a one-step staining method and involves a labeled antibody (e.g. FITC-conjugated antiserum) reacting directly with the antigen. While this technique utilizes only one antibody and therefore is simple and rapid, the sensitivity is lower due to little signal amplification, such as with indirect methods, and is less commonly used than indirect methods. The indirect method involves an unlabeled primary antibody (first layer) that binds to the target antigen in the sample and a labeled secondary antibody (second layer) that reacts with the primary antibody. This method is more sensitive than direct detection strategies because of signal amplification due to the binding of several secondary antibodies to each primary antibody if the secondary antibody is conjugated to the fluorescent or enzyme reporter.

Further amplification can be achieved if the secondary antibody is conjugated to several biotin molecules, which can recruit complexes of avidin-, streptavidin or Neutravidin-enzyme. The indirect method, aside from its greater sensitivity, also has the advantage that only a relatively small number of standard conjugated (labeled) secondary antibodies needs to be generated. With the direct method, it would be necessary to label each primary antibody for every antigen of interest. It must be borne in mind that immunochemistry techniques can also be used to detect certain nucleic acid sequences if a tagged nucleic acid probe (designed to specifically bind to a certain target nucleic acid sequence) can later on be detected with a labelled antibody. Thus, the detection of the protein could be performed by using a tagged nucleic acid designed to bind a specific sequence of the target protein RNA, and then detecting said tagged nucleic acid with a labelled antibody which selectively binds to the tag.

Immunoassay procedures suitable include enzyme-linked immunosorbent assays (ELISA, such as multiplex ELISA), enzyme immunodot assay, agglutination assay, antibody-antigen-antibody sandwich assay, antigen-antibody-antigen sandwich assay, immunocromatography, or other immunoassay formats well-known to the ordinarily skilled artisan, such as radioimmunoassay, chemoluminiscent assays, lateral flow assays or electrochemical assay, as well as protein microarray formats.

Thus, in some embodiments of any of the aspects above or below, the level of expression of protein is determined by an immunoassay. In another embodiment, in combination with any of the embodiments provided above or below, the level of expression of protein is determined by ELISA; more in particular a multiplex ELISA.

Alternatively, the level of expression of a protein can be determined by bioluminescence, fluorescence, chemiluminescence, electrochemistry, or mass spectrometry.

Alternatively, the level of expression of protein can be determined by measuring the levels of proteotypic peptides of the protein (peptides with an amino acid sequence uniquely associated with the studied protein in a given proteome) by mass spectrometry.

As previously indicated, in some embodiment of any of the aspects of the invention, in combination with any of the embodiments provided above or below, the level of expression of protein is determined using an antibody or a fragment thereof able to bind to the target protein(s). These antibodies can be used as “means” for determining the expression of the target proteins in the kits of the invention. Next paragraphs illustrate in more detail this option.

The term “antibody or a fragment thereof able to bind to the target protein(s)” is to be understood as any immunoglobulin or fragment thereof able to selectively bind the target protein. It includes monoclonal and polyclonal antibodies. The term “fragment thereof encompasses any part of an antibody having the size and conformation suitable to bind an epitope of the target protein. Suitable fragments include F(ab), F(ab′) and Fv. An “epitope” is the part of the antigen being recognized by the immune system (B-cells, T-cells or antibodies).

The antibodies used for specific detection can be polyclonal or monoclonal. There are well known means in the state of the art for preparing and characterizing antibodies. Methods for generating polyclonal antibodies are well known in the prior art. Briefly, one prepares polyclonal antibodies by immunizing an animal with the protein; then, serum from the immunized animal is collected and the antibodies isolated. A wide range of animal species can be used for the production of the antiserum. Typically, the animal used for production of antisera can be a rabbit, mouse, rat, hamster, guinea pig or goat.

Moreover, monoclonal antibodies (MAbs) can be prepared using well-known techniques. Typically, the procedure involves immunizing a suitable animal with the protein associated with the disease. The immunizing composition can be administered in an amount effective to stimulate antibody producing cells. Methods for preparing monoclonal antibodies are initiated generally following the same lines as the polyclonal antibody preparation. The immunogen is injected into animals as antigen. The antigen may be mixed with adjuvants such as complete or incomplete Freund's adjuvant. At intervals of two weeks, approximately, the immunization is repeated with the same antigen.

The antibody or fragment thereof for detecting the target protein(s) (i.e., MDK and optionally one or more of the proteins listed) can be included in a kit. The kit may additionally comprise other means (additives, solvents) to visualize the antibody-protein interactions.

Alternatively, the level of expression of MDK and if determined of one or more of the above-listed proteins, is determined at the mRNA level. Next paragraphs illustrate in more detail this option.

Thus, in another embodiment of the kits or their use, the kit is a microarray.

In another embodiment, the kit is a microarray including a defined set of genes encoding protein endometrial cancer markers. All the embodiments provided above for particular proteins to be analyzed (from two to eleven of the list), whose expression is significantly altered by endometrial disease, are also particular embodiments of microarrays.

Additionally, the kits of the invention comprise reagents for detecting a protein encoded by a constitutive gene. The availability of said additional reagents allows normalizing the measurements performed in different samples (for example, the sample to be analysed and the control sample) to rule out that the differences in the expression of the biomarkers are due to a different quantity of total protein amount in the sample more than the real differences in the relative levels of expression. The constitutive genes in the present invention are genes that are always active or being transcribed constantly and which encode for proteins that are expressed constitutively and carry out essential cellular functions. Proteins that are expressed constitutively and can be used in the present invention include, without limitation, 3-2-microglobulin (B2M), ubiquitin, 18-S ribosomal protein, cyclophilin, GAPDH, PSMB4, tubulin and actin.

In one embodiment, the amount of mRNA of each one of the markers are detected via polymerase chain reaction using, for example, oligonucleotide primers that hybridize to one or more polynucleotide endometrial cancer markers or complements of such polynucleotides. Within other embodiments, the amount of mRNA is detected using a hybridization technique, employing oligonucleotide probes that hybridize to one or more polynucleotide endometrial cancer markers or complements of such polynucleotides.

When using mRNA detection, the methods of the invention may be carried out by combining isolated mRNA with reagents to convert to cDNA according to standard methods well known in the art, treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more of the polynucleotide endometrial cancer markers in the sample. For mRNA, the analyzing step may be accomplished using Northern Blot analysis to detect the presence of polynucleotide endometrial cancer markers in the sample. The analysis step may be further accomplished by quantitatively detecting the presence of polynucleotide endometrial cancer markers in the amplification product, and comparing the quantity of marker detected against a panel of expected values for the known presence or absence of such markers in normal and malignant tissue derived using similar primers.

In another embodiment, the invention provides a method wherein mRNA is detected by: (a) isolating mRNA from a sample and combining the mRNA with reagents to convert it to cDNA; (b) treating the converted cDNA with amplification reaction reagents and nucleic acid primers that hybridize to one or more of the polynucleotide endometrial cancer markers endometrial cancer marker to produce amplification products; (c) analyzing the amplification products for determining the amount of mRNA present encoding the protein endometrial cancer marker; and (d) comparing the determined amount of mRNA to an amount detected against a panel of expected values for normal and diseased tissue (e.g., malignant tissue) derived using similar methods.

In particular embodiments of the invention, RT-PCR can be used to amplify the mRNA for protein endometrial cancer markers for detection and analysis. Other embodiments of the invention use quantitative RT-PCR to quantitatively determine amount of mRNA for protein endometrial cancer markers. Further embodiments of the invention use real time RT-PCR for quantification and analysis.

UniprotKB (https://www.uniprot.org/help/uniprotkb) database accession numbers of all the herewith listed proteins or in the corresponding Tables of this description correspond to versions (database release) accessible on Jul. 23, 2021.

AGRIN has the Uniprot database accession number 000468. This protein is a heparin sulfate basal lamina glycoprotein involved in the formation and the maintenance of the neuromuscular junction.

PIGR, also known as polymeric immunoglobulin receptor, has the Uniprot database accession number P01833, Jun. 26, 2007—v4. This receptor binds polymeric IgA and IgM at the basolateral surface of epithelial cells.

CD44, also known as CD44 antigen, has the Uniprot database accession number P16070, Oct. 5, 2010—v3. Mediates cell-cell and cell-matrix interactions through its affinity for HA, and possibly also through its affinity for other ligands such as osteopontin, collagens, and matrix metalloproteinases (MMPs).

LDHA, also known as L-lactate dehydrogenase A chain, has the Uniprot database accession number P00338, Jan. 23, 2007—v2. This protein is involved in step 1 of the subpathway that synthesizes (S)-lactate from pyruvate.

Some of these proteins have been related with EC grading/diagnosis in tissue samples. However, no meaningful data are correlated with values in a sample from the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, a uterine fluid, uterine washings and combinations thereof. As above exposed, detection of markers in these kinds of samples imply the advantage of collecting data from routine sampling in clinical practice of the gynecological diseases, avoiding costs and importantly, invasive tissue biopsies.

A further aspect of the present invention was a method of deciding or recommending whether to initiate a medical regimen of a subject suffering endometrial cancer, in particular in function of the prognosis. In a particular embodiment of this method, it comprises
a) determining, in vitro, the presence and/or the level of expression of midkine (MDK) in a sample from the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, a uterine fluid, uterine washings and combinations thereof; and
b) comparing the level of MDK of step (a) with a corresponding reference value or reference interval for each protein, said reference value or interval selected from a value or interval of values from a subject suffering from endometrial cancer, and/or comparing with a cut-off value discriminating among endometrial cancer and other gynecological disorders or conditions, and
c) wherein the subject is diagnosed of endometrial cancer if the level of MDK is within the value or interval of values from a subject suffering from this cancer, and/or if the level of MDK in relation to the cut-off value is classified to the endometrial cancer group, in such a way that:
   i) if the subject is diagnosed of suffering from endometrial carcinoma, or of being suspicious of suffering from endometrial carcinoma, then the initiation of the medical regimen is recommended, which consists of total hysterectomy and bilateral salpingo-oophorectomy, optionally complemented with pelvic and para-aortic lymphadenectomy, and omentectomy;
   ii) if the patient is diagnosed of not suffering from endometrial carcinoma, the follow-up is performed optionally in consideration of the result of an examination of the patient by a physician.

In another particular embodiment method of deciding or recommending whether to initiate a medical regimen of a subject suffering endometrial cancer, usually in function of the established prognosis, an adjuvant treatment is recommended selected from radiotherapy, brachyterapy, hormone therapy, chemotherapy, targeted therapies and combinations thereof.

This method of deciding or recommending whether to initiate a medical regimen of a subject suffering endometrial cancer is also applicable to the previously disclosed methods for the diagnosis of recurrence or risk of recurrence of endometrial cancer; and/or for the diagnosis of endometrial cancer subtype and/or endometrial cancer molecular classification.

Thus, herewith disclosed is also a method for the treatment of EC in a subject suffering from this disease, the method comprising carrying out the method of the diagnosis and/or prognosis of the first aspect or of any one of its embodiments; and further comprising the step of treating the subject by means of total hysterectomy and/or bilateral salpingo-oophorectomy, optionally complemented with pelvic and para-aortic lymphadenectomy, and/or omentectomy. In a particular embodiment of the proposed method of treatment, the subject also receives an adjuvant treatment is recommended selected from radiotherapy, brachyterapy, hormone therapy, chemotherapy, targeted therapies and combinations thereof.

Also disclosed is, moreover, a method of detecting, in an isolated sample of a subject, the level of MDK, and/or optionally one or more of the proteins listed in Table 1, the method comprising:
(a) obtaining a sample from the subject, said sample from the female genital tract part including one or more of the vulvae, vagina, cervix, uterus, fallopian tubes, and ovaries and selected from a gynecologic sampling, including a cervical fluid, a cytology, a pap-smear sample, an endometrial biopsy, an uterine fluid, uterine washings and combinations thereof; and
(b) detecting the presence and/or the level of expression of midkine (MDK) in the isolated sample by: (i) contacting said sample with means capable of binding the corresponding protein and detecting said binding; or (ii) contacting said sample with means capable of binding corresponding RNA going to be translated to the said protein and detecting said binding.

Another aspect of present invention is to provide an algorithm for carrying out any of the methods of diagnosis and/or of prognosis as defined in the above aspects and embodiments, or for carrying out any of the methods disclosed above for the diagnosis of recurrence or risk of recurrence of endometrial cancer and/or endometrial cancer subtype and/or endometrial cancer molecular classification.

As indicated, this fourth aspect relates to computer-implemented methods for carrying out the method as defined in the first aspect, in which after the determination of the level of expression of MDK, and optionally of the one or more of the proteins for the diagnosis and/or for the prognosis of endometrial cancer, said level(s) are given a value and/or a score, and optionally are computed in a mathematical formula to obtain a computed value; wherein in function of the said level(s), score(s) and or computed value(s), a decision is taken between the options of suffering or not from EC and/or between the options of suffering among EC presenting different prognosis, including different histological subtypes and grades and different molecular features.

In a particular embodiment the algorithm is a computer-implemented method for diagnosing EC and/or for prognosing EC, in particular for the prognosis of the disease by determining EC subtype, in particular EEC or NEEC. This algorithm allows taking the decision of a sample being from a subject suffering from EC or not; and also if a sample being from a subject suffering from EC is suffering from EEC or from NEEC. In a more particular embodiment, the algorithm provides with recommended treatment. Therefore, there is also provided a computer-implemented method for carrying out the method as defined above, in which after the determination of the level of expression of one or more of the proteins for the diagnosis and/or for the prognosis of EC, said level(s) are given a value and/or a score, and optionally are computed in a mathematical formula to obtain a computed value; wherein in function of the said level(s), score(s) and or computed value(s), a decision is taken between the options of suffering or not from EC and/or between the options of suffering among different EC subtypes.

In other words, in another particular embodiment of the algorithm of the invention, after the determination of the level of expression of one or more of the proteins for the diagnosis and/or for the prognosis of EC, said level(s) are given a value and/or a score, and optionally are computed in a mathematical formula to obtain a computed value; wherein in function of the said level(s), score(s) and or computed value(s), a decision is taken between the options of suffering or not from EC and/or between the options of suffering among different EC subtypes.

In a particular embodiment the algorithm is a computer-implemented method for diagnosing EC and/or for prognosing EC, in particular for the prognosis of the disease by determining EC grading, in particular low or high grade EC, according to known gradation or staging systems, such as the one of FIGO. In another particular embodiment the algorithm is a computer-implemented method for diagnosing EC and/or for prognosing EC, in particular for the prognosis of the disease by determining the EC hystological subtype, such as endometrioid vs non-endometrioid endometrial cancer. This algorithm allows taking the decision of a sample being from a subject suffering from EC or not; and also if a sample being from a subject suffering from EC is suffering from low or from high grade EC. In a more particular embodiment, the algorithm provides with recommended treatment. Therefore, there is also provided a computer-implemented method for carrying out the method as defined above, in which after the determination of the level of expression of one or more of the proteins for the diagnosis and/or for the prognosis of EC, said level(s) are given a value and/or a score, and optionally are computed in a mathematical formula to obtain a computed value; wherein in function of the said level(s), score(s) and or computed value(s), a decision is taken between the options of suffering or not from EC and/or between the options of suffering among different EC grades.

In other words, in another particular embodiment of the algorithm of the invention, after the determination of the level of expression of one or more of the proteins for the diagnosis and/or for the prognosis of EC, said level(s) are given a value and/or a score, and optionally are computed in a mathematical formula to obtain a computed value; wherein in function of the said level(s), score(s) and or computed value(s), a decision is taken between the options of suffering or not from EC and/or between the options of suffering among different EC grades.

The in vitro methods of the invention provide diagnostic and/or prognostic information. In one embodiment, the methods of the invention further comprise the steps of (i) collecting the diagnostic and/or prognostic information, and (ii) saving the information in a data carrier.

In the sense of the invention a “data carrier” is to be understood as any means that contain meaningful information data for the diagnosis and/or prognosis of endometrial carcinoma, such as paper. The carrier may also be any entity or device capable of carrying the prognosis data. For example, the carrier may comprise a storage medium, such as a ROM, for example a CD ROM or a semiconductor ROM, or a magnetic recording medium, for example a floppy disc or hard disk. Further, the carrier may be a transmissible carrier such as an electrical or optical signal, which may be conveyed via electrical or optical cable or by radio or other means. When the diagnosis/prognosis data are embodied in a signal that may be conveyed directly by a cable or other device or means, the carrier may be constituted by such cable or other device or means. Other carriers relate to USB devices and computer archives. Examples of suitable data carrier are paper, CDs, USB, computer archives in PCs, or sound registration with the same information.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### Example 1

### Discovery Cohort. Validation Cohort and Analysis of the Information of the Markers. Informative Data from Analysis of MDK Levels in Cervical Fluid Samples (Routine Non-Invasive Gynecological Sampling)

Materials and Methods: The discovery phase to identify potential biomarkers using the fluid contained in pap-smears, i.e. cervical fluid samples was performed using a shotgun label-free proteomic approach. The study included 60 patients (20 EC patients, 20 controls suffering AVB without endometrial cancer or cervical pathology, and 20 controls without endometrial pathology but cervical pathology). The data was analyzed using MaxQuant and R software. The levels of 110 peptides corresponding to 75 proteins identified in the discovery phase, were further measured in a verification phase of 242 (106 non-EC, 129 EC, 7 premalignancies of EC) patients by mass spectrometry (LC-PRM). Analysis was performed using Skyline software, SPSS and R software. The clinicopathological features of the patients included in the verification study are shown in Table 2.

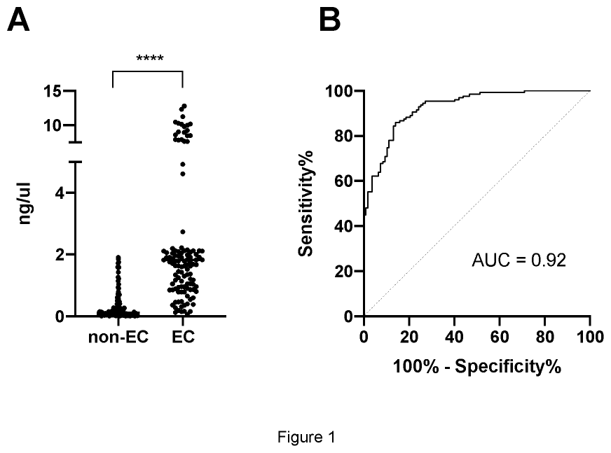

Results: The discovery study permitted to determine a total number of 2,888 proteins identified with more than a single peptide in our samples. Statistical analysis permitted to identify 75 potential proteins differently expressed between EC and non-EC patients to be further assessed and verified. Verification phase revealed the great potential of 60 of those proteins measured in the cervical samples to reach a non-invasive diagnosis of EC comparing EC vs non-EC patients (adj.p.value<0.05, Fold-Change>2, AUC>0.7) (Table 3). Specifically, 19 proteins achieved an AUC>0.75, and 7 proteins an AUC>0.8. Additionally, an ELISA assay of the best performing protein was tested in all the samples reproducing the results obtained by mass-spectrometry and reaching an AUC=0.91 in this dataset to diagnose EC (comparing 127 EC vs 106 non-EC patients) (FIG. 1). The verification study allowed to identify a set of significant proteins as biomarkers of prognosis to differentiate between histological subtypes and grades of EC patients. Specifically, a set 34 protein biomarkers had a significant adjusted p-value (Table 3, in bold) and 39 proteins a fold-change higher than 1.3 (Table 3, shadowed) when comparing the 35 low grade (G1) vs. the 37 high grade (G3) EC patients. A comparison between low and intermediate grades, G1 and G2, against high grade (G3) was also performed. Significant and highly-expressed proteins are illustrated in Table 3. Regarding histological subtype, a set of 33 proteins were differentially expressed (fold-change higher than 1.3) and 31 proteins were identified as significant (adj. p-value <0.05) when comparing 102 endometrioid endometrial cancer (EEC) against 25 non-endometrioid endometrial cancer patients. These results are all shown in Table 3. Tables 8 and 9 illustrate also the 2 or 3 5 protein panels that in combination allowed to differentiate between histological subtypes and grades of EC patients.

Next Table 3 shows AUC, SE and SP of MDK an other markers. Note that MDK provided the best values.

TABLE 2
Clinicopathological features of the patients
included in the verification phase.

| | EC | non-EC | Hyperplasia |
|---|---|---|---|
| | (n = 129) | (n = 106) | (n = 7) |
| Age (years) | | | |
| Mean | 64 | 64 | 50 |
| Minimum | 32 | 31 | 44 |
| Maximum | 92 | 95 | 55 |
| Uterine condition | | | |
| Premenopausal | 29 | 13 | 5 |
| Postmenopausal | 100 | 90 | 2 |
| Histological type | | | |
| Endometrioid | 103 | | |
| Serous | 9 | | |
| Others (carcinosarcoma) | 16 | | |
| Histological grade | | | |
| Grade 1 | 36 | | |
| Grade 2 | 56 | | |
| Grade 3 | 37 | | |
| FIGO stage | | | |
| IA | 62 | | |
| IB | 19 | | |
| II | 26 | | |
| IIIA | 4 | | |
| IIIB | 2 | | |
| IIIC1 | 2 | | |
| IIIC2 | 7 | | |
| IVA | 2 | | |
| IVB | 5 | | |
| Miometrial invasion | | | |
| <50% | 80 | | |
| >50% | 49 | | |
| Lymphobascular invasion | | | |
| Yes | 35 | | |
| No | 84 | | |

TABLE 3
1-protein biomarker detection for EC diagnosis and prognosis, including the differentiation between
histological grades and subtypes of ECs. Significant proteins with adjusted p-value < 0.05 are in
bold, and significant proteins with fold-change (FC) higher than 1.3 are shown with a ¹ symbol.

| | | | | Prognostic biomarkers | | | Prognostic | | | Prognostic biomarkers | | |
|---|---|---|---|---|---|---|---|---|---|---|---|---|
| | Diagnostic | | | Histological grade | | | biomarkers | | | Histological subtype | | |
| | biomarkers (EC | | | (Low G1 vs High G3 | | | (G1G2 vs High | | | (Endometrioid vs | | |
| | vs non-EC) | | | grade) | | | G3 grade) | | | Non-Endometrioid ECs) | | |
| | FC | adj. p | AUC | FC | adj. p | AUC | FC | adj. p | AUC | FC | adj. p | AUC |
| A2ML1 | 0.42 | 0.00 | 0.63 | 0.69 | 0.06 | 0.59 | 0.89 | 0.84 | 0.51 | 0.84 | 0.74 | 0.52 |
| AGRN | 2.32¹ | 0.00¹ | 0.72¹ | 2.90¹ | 0.01¹ | 0.65¹ | 1.67¹ | 0.11¹ | 0.59¹ | 1.75¹ | 0.06¹ | 0.61¹ |
| ANXA3 | 0.55 | 0.00 | 0.69 | 0.61 | 0.03 | 0.67 | 0.69 | 0.06 | 0.64 | 0.73 | 0.35 | 0.58 |
| ANXA7 | 0.52 | 0.00 | 0.72 | 1.68¹ | 0.12¹ | 0.62¹ | 1.49¹ | 0.23¹ | 0.59¹ | 1.73¹ | 0.15¹ | 0.61¹ |
| APOB | 3.02¹ | 0.00¹ | 0.81¹ | 2.25¹ | 0.00¹ | 0.76¹ | 1.49¹ | 0.03¹ | 0.66¹ | 1.69¹ | 0.03¹ | 0.68¹ |
| APOC1 | 1.96¹ | 0.00¹ | 0.77¹ | 1.49¹ | 0.03¹ | 0.67¹ | 1.22 | 0.25 | 0.59 | 1.21 | 0.26 | 0.59 |
| APOF | 2.20¹ | 0.00¹ | 0.77¹ | 2.08¹ | 0.00¹ | 0.74¹ | 1.40¹ | 0.02¹ | 0.67¹ | 1.76¹ | 0.02¹ | 0.70¹ |
| APP | 2.85¹ | 0.00¹ | 0.79¹ | 1.68¹ | 0.08¹ | 0.64¹ | 0.54 | 0.34 | 0.56 | 0.99 | 0.40 | 0.57 |
| ATP6V1G1 | 0.94 | 0.33 | 0.54 | 1.32¹ | 0.41¹ | 0.57¹ | 1.30 | 0.45 | 0.55 | 1.51¹ | 0.15¹ | 0.61¹ |
| BTD | 1.45¹ | 0.00¹ | 0.71¹ | 1.06 | 0.89 | 0.51 | 0.98 | 0.88 | 0.51 | 1.08 | 0.73 | 0.53 |
| C1QA | 2.15¹ | 0.00¹ | 0.81¹ | 1.57¹ | 0.00¹ | 0.75¹ | 1.18 | 0.09 | 0.62 | 1.33¹ | 0.10¹ | 0.62¹ |
| C1R | 1.89¹ | 0.00¹ | 0.75¹ | 1.62¹ | 0.00¹ | 0.76¹ | 1.20 | 0.08 | 0.63 | 1.41¹ | 0.05¹ | 0.65¹ |
| C4BPA | 2.14¹ | 0.00¹ | 0.75¹ | 2.36¹ | 0.00¹ | 0.79¹ | 1.52¹ | 0.03¹ | 0.66¹ | 1.78¹ | 0.02¹ | 0.69¹ |
| CA1 | 0.89 | 0.00 | 0.61 | 1.39¹ | 0.35¹ | 0.58¹ | 1.02 | 0.51 | 0.55 | 1.63¹ | 0.38¹ | 0.57¹ |
| CALU | 1.10 | 0.01 | 0.60 | 1.50¹ | 0.01¹ | 0.70¹ | 1.16 | 0.25 | 0.58 | 1.31¹ | 0.04¹ | 0.66¹ |
| CAMP | 0.85 | 0.62 | 0.52 | 0.58 | 0.08 | 0.64 | 0.76 | 0.16 | 0.60 | 0.95 | 0.82 | 0.52 |
| CAPS | 1.49¹ | 0.00¹ | 0.62¹ | 0.53 | 0.73 | 0.53 | 0.57 | 0.19 | 0.60 | 0.69 | 0.22 | 0.60 |
| CCT6A | 1.94¹ | 0.00¹ | 0.76¹ | 1.55¹ | 0.06¹ | 0.65¹ | 1.23 | 0.33 | 0.58 | 1.67¹ | 0.05¹ | 0.65¹ |
| CD44 | 0.99 | 0.30 | 0.54 | 1.30¹ | 0.36¹ | 0.58¹ | 1.19 | 0.84 | 0.52 | 1.02 | 0.75 | 0.53 |
| CHMP4B | 0.69 | 0.01 | 0.61 | 1.67¹ | 0.12¹ | 0.62¹ | 1.57¹ | 0.16¹ | 0.60¹ | 1.65¹ | 0.22¹ | 0.60¹ |
| COL12A1 | 3.93¹ | 0.00¹ | 0.75¹ | 1.02 | 0.40 | 0.57 | 0.52 | 0.72 | 0.53 | 0.52 | 0.60 | 0.54 |
| COL1A1 | 4.30¹ | 0.00¹ | 0.83¹ | 1.37¹ | 0.41¹ | 0.56¹ | 1.31¹ | 0.36¹ | 0.57¹ | 1.35¹ | 0.41¹ | 0.57¹ |
| COL3A1 | 2.23¹ | 0.00¹ | 0.69¹ | 1.25 | 0.36 | 0.57 | 1.46¹ | 0.14¹ | 0.60¹ | 1.63¹ | 0.24¹ | 0.59¹ |
| CSE1L | 1.71¹ | 0.00¹ | 0.65¹ | 2.16¹ | 0.01¹ | 0.70¹ | 1.75¹ | 0.06¹ | 0.63¹ | 2.25¹ | 0.00¹ | 0.74¹ |
| CTNNB1 | 1.11 | 0.01 | 0.60 | 1.60¹ | 0.08¹ | 0.64¹ | 1.13 | 0.65 | 0.53 | 1.28 | 0.22 | 0.59 |
| EPS8L1 | 0.58 | 0.00 | 0.65 | 1.12 | 0.95 | 0.51 | 1.09 | 0.82 | 0.52 | 1.09 | 0.88 | 0.51 |
| FAM107B | 0.59 | 0.00 | 0.68 | 0.79 | 0.36 | 0.57 | 0.84 | 0.41 | 0.56 | 0.86 | 0.53 | 0.55 |
| FCGBP | 0.34 | 0.00 | 0.69 | 0.36 | 0.00 | 0.78 | 0.47 | 0.01 | 0.70 | 0.44 | 0.02 | 0.68 |
| FGFBP1 | 2.48¹ | 0.00¹ | 0.78¹ | 1.11 | 0.75 | 0.53 | 0.85 | 0.41 | 0.56 | 1.01 | 0.60 | 0.54 |
| FN1 | 2.34¹ | 0.00¹ | 0.81¹ | 1.86¹ | 0.00¹ | 0.75¹ | 1.32¹ | 0.05¹ | 0.65¹ | 1.24 | 0.07 | 0.64 |
| GPLD1 | 1.63¹ | 0.00¹ | 0.71¹ | 1.58¹ | 0.00¹ | 0.73¹ | 1.27 | 0.05 | 0.64 | 1.45¹ | 0.04¹ | 0.66¹ |
| GRN | 0.51 | 0.00 | 0.67 | 0.69 | 0.03 | 0.67 | 0.85 | 0.13 | 0.61 | 0.91 | 0.31 | 0.58 |
| GYG1 | 0.68 | 0.02 | 0.59 | 0.84 | 0.89 | 0.51 | 0.94 | 0.93 | 0.51 | 1.06 | 0.53 | 0.55 |
| HCLS1 | 0.74 | 0.09 | 0.57 | 0.73 | 0.44 | 0.56 | 0.80 | 0.40 | 0.56 | 0.93 | 0.93 | 0.51 |
| HSPE1 | 0.95 | 0.30 | 0.54 | 2.15¹ | 0.00¹ | 0.83¹ | 1.44¹ | 0.01¹ | 0.70¹ | 1.33¹ | 0.02¹ | 0.69¹ |
| IGFALS | 1.68¹ | 0.00¹ | 0.71¹ | 1.30 | 0.08 | 0.64 | 1.10 | 0.41 | 0.56 | 1.20 | 0.35 | 0.58 |
| IMPA1 | 0.70 | 0.00 | 0.66 | 0.99 | 0.90 | 0.51 | 0.99 | 0.89 | 0.51 | 1.10 | 0.42 | 0.56 |
| ITGB2 | 0.82 | 0.48 | 0.53 | 0.79 | 0.39 | 0.57 | 0.89 | 0.51 | 0.55 | 1.08 | 0.74 | 0.53 |
| ITIH2 | 1.88¹ | 0.00¹ | 0.76¹ | 1.43¹ | 0.02¹ | 0.69¹ | 1.17 | 0.14 | 0.61 | 1.28 | 0.11 | 0.62 |
| KRT8 | 1.07 | 0.00 | 0.61 | 2.15¹ | 0.40¹ | 0.57¹ | 1.33¹ | 0.81¹ | 0.52¹ | 0.88 | 0.98 | 0.50 |
| LBP | 2.58¹ | 0.00¹ | 0.76¹ | 2.28¹ | 0.02¹ | 0.68¹ | 1.80¹ | 0.03¹ | 0.66¹ | 2.69¹ | 0.02¹ | 0.71¹ |
| LCAT | 1.89¹ | 0.00¹ | 0.75¹ | 1.71¹ | 0.00¹ | 0.74¹ | 1.26 | 0.05 | 0.65 | 1.48¹ | 0.03¹ | 0.67¹ |
| LCN2 | 0.48 | 0.00 | 0.75 | 0.50 | 0.00 | 0.77 | 0.57 | 0.01 | 0.70 | 0.53 | 0.02 | 0.68 |
| LDHA | 0.96 | 0.58 | 0.52 | 1.32¹ | 0.18¹ | 0.61¹ | 1.17 | 0.45 | 0.56 | 1.43¹ | 0.02¹ | 0.69¹ |
| LGALS1 | 1.14 | 0.03 | 0.59 | 1.65¹ | 0.05¹ | 0.66¹ | 1.40¹ | 0.25¹ | 0.59¹ | 1.74¹ | 0.02¹ | 0.69¹ |
| LMNB1 | 0.86 | 0.62 | 0.52 | 1.18 | 0.08 | 0.64 | 1.08 | 0.34 | 0.57 | 1.22 | 0.09 | 0.63 |
| LMO7 | 0.34 | 0.00 | 0.73 | 0.70 | 0.17 | 0.61 | 0.77 | 0.34 | 0.57 | 0.53 | 0.05 | 0.65 |
| LSP1 | 0.79 | 0.21 | 0.55 | 0.97 | 0.70 | 0.53 | 0.97 | 0.99 | 0.50 | 1.11 | 0.56 | 0.55 |
| MDK | 5.54¹ | 0.00¹ | 0.91¹ | 1.81¹ | 0.14¹ | 0.61¹ | 1.45¹ | 0.41¹ | 0.56¹ | 0.69 | 0.53 | 0.55 |
| MMP8 | 0.74 | 0.30 | 0.54 | 0.88 | 0.18 | 0.61 | 1.02 | 0.32 | 0.58 | 1.21 | 0.82 | 0.52 |
| MUC4 | 0.39 | 0.00 | 0.75 | 0.41 | 0.01 | 0.70 | 0.47 | 0.01 | 0.70 | 0.55 | 0.03 | 0.67 |
| NAMPT | 0.92 | 0.90 | 0.51 | 1.39¹ | 0.07¹ | 0.64¹ | 1.22 | 0.36 | 0.57 | 1.57¹ | 0.03¹ | 0.67¹ |
| NEDD8-MDP1 | 0.71 | 0.00 | 0.66 | 0.99 | 0.56 | 0.55 | 1.03 | 0.84 | 0.52 | 1.21 | 0.49 | 0.56 |
| PEA15 | 0.66 | 0.01 | 0.61 | 1.36¹ | 0.34¹ | 0.58¹ | 1.19 | 0.59 | 0.54 | 1.37¹ | 0.26¹ | 0.59¹ |
| PIGR | 1.11 | 0.30 | 0.54 | 0.17 | 0.00 | 0.91 | 0.23 | 0.00 | 0.83 | 0.20 | 0.00 | 0.85 |
| PKM | 0.85 | 0.07 | 0.57 | 3.89¹ | 0.00¹ | 0.78¹ | 2.45¹ | 0.01¹ | 0.71¹ | 1.79¹ | 0.00¹ | 0.75¹ |
| PLBD1 | 0.49 | 0.00 | 0.68 | 0.72 | 0.09 | 0.63 | 0.80 | 0.18 | 0.60 | 0.85 | 0.53 | 0.55 |
| PPL | 0.39 | 0.00 | 0.72 | 0.79 | 0.21 | 0.60 | 0.84 | 0.40 | 0.56 | 0.71 | 0.10 | 0.62 |
| RAB21 | 0.82 | 0.10 | 0.57 | 1.27 | 0.18 | 0.61 | 1.26 | 0.26 | 0.58 | 1.50¹ | 0.03¹ | 0.67¹ |
| S100A9 | 0.32 | 0.00 | 0.73 | 0.71 | 0.03 | 0.67 | 0.93 | 0.30 | 0.58 | 0.53 | 0.37 | 0.57 |
| SAA2-SAA4; SAA4 | 2.15¹ | 0.00¹ | 0.76¹ | 1.98¹ | 0.00¹ | 0.74¹ | 1.55¹ | 0.02¹ | 0.67¹ | 1.94¹ | 0.02¹ | 0.70¹ |
| SCEL | 0.34 | 0.00 | 0.71 | 0.56 | 0.01 | 0.71¹ | 0.65 | 0.16 | 0.60 | 0.37 | 0.03 | 0.68 |
| SERPINA3 | 1.87¹ | 0.00¹ | 0.69¹ | 1.61¹ | 0.03¹ | 0.67¹ | 1.29 | 0.07 | 0.63 | 1.75¹ | 0.04¹ | 0.66¹ |
| SERPIND1 | 2.51¹ | 0.00¹ | 0.80¹ | 1.82¹ | 0.01¹ | 0.72¹ | 1.30 | 0.06 | 0.64 | 1.60¹ | 0.02¹ | 0.68¹ |
| SERPINH1 | 2.55¹ | 0.00¹ | 0.72¹ | 2.63¹ | 0.00¹ | 0.76¹ | 2.14¹ | 0.01¹ | 0.71¹ | 2.62¹ | 0.00¹ | 0.80¹ |
| SPP1 | 3.49¹ | 0.00¹ | 0.79¹ | 1.34¹ | 0.39¹ | 0.57¹ | 1.28 | 0.41 | 0.56 | 1.39¹ | 0.42¹ | 0.56¹ |
| SPRR1A | 0.32 | 0.00 | 0.73 | 0.34 | 0.01 | 0.71 | 0.46 | 0.03 | 0.66¹ | 0.25 | 0.02 | 0.68 |
| SPRR1B | 0.30 | 0.00 | 0.74 | 0.41 | 0.01 | 0.71 | 0.55 | 0.11 | 0.62 | 0.27 | 0.07 | 0.64 |
| STMN1 | 1.04 | 0.72 | 0.51 | 1.91¹ | 0.06¹ | 0.65¹ | 1.69¹ | 0.13¹ | 0.61¹ | 2.06¹ | 0.02¹ | 0.68¹ |
| STX7 | 0.52 | 0.00 | 0.68 | 0.78 | 0.40 | 0.57 | 0.86 | 0.48 | 0.55 | 0.89 | 0.86 | 0.51 |
| TCP1 | 2.26¹ | 0.00¹ | 0.78¹ | 1.46¹ | 0.12¹ | 0.62¹ | 1.21 | 0.41 | 0.56 | 1.51¹ | 0.05¹ | 0.65¹ |
| TIMP2 | 0.59 | 0.00 | 0.72 | 0.53 | 0.00 | 0.74 | 0.70 | 0.11 | 0.62 | 0.67 | 0.10 | 0.62 |
| TMPRSS11E | 0.52 | 0.00 | 0.70 | 0.29 | 0.00 | 0.75 | 0.34 | 0.06 | 0.63 | 0.12 | 0.02 | 0.69 |
| VIM | 1.69¹ | 0.00¹ | 0.64¹ | 1.08 | 0.08 | 0.64 | 1.01 | 0.57 | 0.54 | 1.28 | 0.11 | 0.62 |
| VWF | 3.38¹ | 0.00¹ | 0.82¹ | 2.41¹ | 0.00¹ | 0.81¹ | 1.52¹ | 0.01¹ | 0.70¹ | 1.78¹ | 0.02¹ | 0.71¹ |

AUC: Area Under the Curve;
FC: Fold Change;
adj. p.: adjusted p-value;
EC: endometrial cancer;
G: Grade

When MDK was combined in a two-pannel and three-pannel of protein biomarkers, the AUC value of the combinations was increased in comparison to MDK alone, mainly by increasing the sensitivity to diagnose EC. Data are illustrated in next Tables 4 and 5. As well, proteins providing significant information on prognosis were combined in Tables 6 and 7. As indicated before, the sensitivity is of importance considering the symptomatology of the disease, common with other disorders of the female genital tract.

TABLE 4
2-protein biomarker detection for EC diagnosis

| Prot 1 | Prot 2 | AUC | Prot 1 | Prot 2 | AUC | Prot 1 | Prot 2 | AUC |
|---|---|---|---|---|---|---|---|---|
| MDK | LCN2 | 0.94 | MDK | TMPRSS11E | 0.92 | MDK | TCP1 | 0.91 |
| MDK | MUC4 | 0.94 | MDK | LMNB1 | 0.92 | MDK | CTNNB1 | 0.91 |
| MDK | MUC4 | 0.94 | MDK | LMNB1 | 0.92 | MDK | CAPS | 0.91 |
| MDK | LCN2 | 0.94 | MDK | SPRR1A | 0.92 | COL1A1 | TCP1 | 0.91 |
| MDK | APOB | 0.94 | MDK | PEA15 | 0.92 | COL1A1 | CCT6A | 0.90 |
| MDK | APOB | 0.94 | MDK | VIM | 0.92 | TCP1 | ANXA7 | 0.90 |
| MDK | FN1 | 0.94 | MDK | GYG1 | 0.92 | CCT6A | ANXA7 | 0.90 |
| MDK | VWF | 0.94 | MDK | IGFALS | 0.92 | COL1A1 | TCP1 | 0.90 |
| MDK | APOF | 0.93 | MDK | MMP8 | 0.92 | COL1A1 | CCT6A | 0.89 |
| MDK | PPL | 0.93 | MDK | ITGB2 | 0.92 | CCT6A | ANXA7 | 0.89 |
| MDK | SAA4 | 0.93 | MDK | RAB21 | 0.92 | APP | FGFBP1 | 0.89 |
| MDK | VWF | 0.93 | MDK | CAMP | 0.92 | VWF | FGFBP1 | 0.89 |
| MDK | FCGBP | 0.93 | MDK | FGFBP1 | 0.91 | TCP1 | ANXA7 | 0.89 |
| MDK | LMO7 | 0.93 | MDK | LSP1 | 0.91 | COL1A1 | FGFBP1 | 0.89 |
| MDK | SERPIND1 | 0.93 | MDK | PKM | 0.91 | APP | LCN2 | 0.89 |
| MDK | STX7 | 0.93 | MDK | COL12A1 | 0.91 | APOB | APP | 0.89 |
| MDK | FCGBP | 0.93 | MDK | APP | 0.91 | C1QA | SPP1 | 0.89 |
| MDK | ANXA7 | 0.93 | MDK | CHMP4B | 0.91 | COL1A1 | FGFBP1 | 0.89 |
| MDK | ITIH2 | 0.93 | MDK | HSPE1 | 0.91 | APOB | SPP1 | 0.89 |
| MDK | C1QA | 0.93 | MDK | BTD | 0.91 | TCP1 | PEA15 | 0.88 |
| MDK | ITIH2 | 0.93 | MDK | A2ML1 | 0.91 | APOB | SPP1 | 0.88 |
| MDK | S100A9 | 0.93 | MDK | SERPINA3 | 0.91 | CCT6A | PEA15 | 0.88 |
| MDK | LBP | 0.93 | MDK | CA1 | 0.91 | VWF | FGFBP1 | 0.88 |
| MDK | APOF | 0.93 | MDK | TMPRSS11E | 0.91 | VWF | APP | 0.88 |
| MDK | ANXA3 | 0.93 | MDK | NAMPT | 0.91 | SERPIND1 | APP | 0.88 |
| MDK | IMPA1 | 0.93 | MDK | CALU | 0.91 | APP | LCN2 | 0.88 |
| MDK | TIMP2 | 0.93 | MDK | FGFBP1 | 0.91 | C1QA | SPP1 | 0.88 |
| MDK | C4BPA | 0.93 | MDK | SERPINH1 | 0.91 | COL1A1 | CSE1L | 0.88 |
| MDK | APOC1 | 0.93 | MDK | MDK | 0.91 | FN1 | FGFBP1 | 0.88 |
| MDK | SERPIND1 | 0.93 | MDK | COL3A1 | 0.91 | APOB | APP | 0.88 |
| MDK | LCAT | 0.93 | MDK | LDHA | 0.91 | COL1A1 | C1QA | 0.88 |
| MDK | SCEL | 0.93 | MDK | STMN1 | 0.91 | SERPIND1 | APP | 0.88 |
| MDK | C4BPA | 0.92 | MDK | HSPE1 | 0.91 | C1QA | APP | 0.88 |
| MDK | EPS8L1 | 0.92 | MDK | LGALS1 | 0.91 | APP | TCP1 | 0.88 |
| MDK | CD44 | 0.92 | MDK | CCT6A | 0.91 | COL1A1 | APOB | 0.88 |
| MDK | PLBD1 | 0.92 | MDK | AGRN | 0.91 | APOB | FGFBP1 | 0.88 |
| MDK | LCAT | 0.92 | MDK | SERPINH1 | 0.91 | APP | MUC4 | 0.88 |
| MDK | APP | 0.92 | MDK | LDHA | 0.91 | FN1 | FGFBP1 | 0.88 |
| MDK | FAM107B | 0.92 | MDK | CCT6A | 0.91 | COL1A1 | SERPINH1 | 0.88 |
| MDK | TIMP2 | 0.92 | MDK | TCP1 | 0.91 | APOB | SPP1 | 0.88 |
| MDK | CD44 | 0.92 | MDK | CALU | 0.91 | APP | FGFBP1 | 0.88 |
| MDK | SPRR1B | 0.92 | MDK | AGRN | 0.91 | COL1A1 | APOB | 0.88 |
| MDK | GRN | 0.92 | MDK | KRT8 | 0.91 | VWF | TCP1 | 0.87 |
| MDK | COL1A1 | 0.92 | MDK | STMN1 | 0.91 | FN1 | FGFBP1 | 0.87 |
| MDK | PIGR | 0.92 | MDK | CSE1L | 0.91 | APP | APOF | 0.87 |
| MDK | NEDD8-MDP1 | 0.92 | MDK | PKM | 0.91 | COL1A1 | TIMP2 | 0.87 |
| MDK | GPLD1 | 0.92 | MDK | VIM | 0.91 | APOB | SPP1 | 0.87 |
| MDK | SPRR1B | 0.92 | MDK | LGALS1 | 0.91 | VWF | CCT6A | 0.87 |
| MDK | SPP1 | 0.92 | MDK | ATP6V1G1 | 0.91 | FN1 | TCP1 | 0.87 |
| MDK | PIGR | 0.92 | MDK | CAPS | 0.91 | MUC4 | AGRN | 0.87 |
| MDK | HCLS1 | 0.92 | MDK | KRT8 | 0.91 | VWF | SPP1 | 0.87 |
| MDK | SPP1 | 0.92 | MDK | CSE1L | 0.91 | FN1 | APP | 0.87 |
| MDK | C1R | 0.92 | MDK | TCP1 | 0.91 | FN1 | SPP1 | 0.87 |

Prot.: Protein, AUC: Area Under the Curve

TABLE 5
3-protein biomarker detection for EC diagnosis

| Prot 1 | Prot 2 | Prot 3 | AUC | Prot 1 | Prot 2 | Prot 3 | AUC |
|---|---|---|---|---|---|---|---|
| MDK | FN1 | FCGBP | 0.953 | MDK | FN1 | LMNB1 | 0.945 |
| MDK | FN1 | LCN2 | 0.952 | MDK | FN1 | LMO7 | 0.945 |
| MDK | APOF | MUC4 | 0.952 | MDK | APOC1 | MUC4 | 0.945 |
| MDK | FN1 | FCGBP | 0.952 | MDK | SAA4 | MUC4 | 0.945 |
| MDK | FN1 | MUC4 | 0.951 | MDK | ITIH2 | MUC4 | 0.945 |
| MDK | ANXA7 | CSE1L | 0.951 | MDK | ANXA7 | SERPINH1 | 0.945 |
| MDK | FN1 | LCN2 | 0.951 | MDK | ANXA7 | FCGBP | 0.945 |
| MDK | FN1 | MUC4 | 0.950 | MDK | ITIH2 | MUC4 | 0.945 |
| MDK | APOB | MUC4 | 0.950 | MDK | MUC4 | CTNNB1 | 0.945 |
| MDK | APOB | MUC4 | 0.950 | MDK | COL1A1 | FCGBP | 0.944 |
| MDK | FN1 | LCN2 | 0.950 | MDK | FN1 | GRN | 0.944 |
| MDK | APOB | MUC4 | 0.950 | MDK | APOF | LCN2 | 0.944 |
| MDK | FN1 | FCGBP | 0.949 | MDK | FN1 | ANXA7 | 0.944 |
| MDK | FN1 | MUC4 | 0.949 | MDK | FN1 | CD44 | 0.944 |
| MDK | FN1 | FCGBP | 0.949 | MDK | LMO7 | FCGBP | 0.944 |
| MDK | VWF | FCGBP | 0.949 | MDK | MUC4 | TMPRSS11E | 0.944 |
| MDK | FN1 | LCN2 | 0.949 | MDK | APOB | LMO7 | 0.944 |
| MDK | FN1 | MUC4 | 0.949 | MDK | FN1 | ANXA3 | 0.944 |
| MDK | APOF | MUC4 | 0.949 | MDK | LCN2 | PPL | 0.944 |
| MDK | LCN2 | MUC4 | 0.949 | MDK | LCN2 | ANXA7 | 0.944 |
| MDK | LCN2 | MDK | 0.949 | MDK | APOB | LMO7 | 0.944 |
| MDK | SERPIND1 | MUC4 | 0.949 | MDK | APOB | IMPA1 | 0.944 |
| MDK | COL1A1 | LCN2 | 0.949 | MDK | MUC4 | SPRR1B | 0.944 |
| MDK | APOB | MUC4 | 0.948 | MDK | COL1A1 | APOB | 0.944 |
| MDK | APOB | LCN2 | 0.948 | MDK | APP | LCN2 | 0.944 |
| MDK | LCN2 | MUC4 | 0.948 | MDK | MUC4 | TIMP2 | 0.944 |
| MDK | VWF | FCGBP | 0.948 | MDK | MUC4 | CSE1L | 0.944 |
| MDK | APOB | LCN2 | 0.948 | MDK | LMO7 | FCGBP | 0.944 |
| MDK | APOF | FCGBP | 0.948 | MDK | APOB | FN1 | 0.944 |
| MDK | MUC4 | APP | 0.948 | MDK | FN1 | CD44 | 0.944 |
| MDK | APOB | LCN2 | 0.948 | MDK | SERPIND1 | FCGBP | 0.944 |
| MDK | MUC4 | FGFBP1 | 0.948 | MDK | FN1 | FGFBP1 | 0.944 |
| MDK | COL1A1 | LCN2 | 0.948 | MDK | APOB | SPRR1B | 0.944 |
| MDK | MUC4 | CCT6A | 0.948 | MDK | APOB | EPS8L1 | 0.944 |
| MDK | MUC4 | APP | 0.948 | MDK | ANXA7 | MUC4 | 0.944 |
| MDK | PPL | MUC4 | 0.947 | MDK | SPP1 | LCN2 | 0.944 |
| MDK | LCN2 | APP | 0.947 | MDK | MUC4 | TIMP2 | 0.944 |
| MDK | SPP1 | MUC4 | 0.947 | MDK | MUC4 | KRT8 | 0.944 |
| MDK | APOB | ANXA7 | 0.947 | MDK | MUC4 | PLBD1 | 0.944 |
| MDK | APOB | ANXA7 | 0.947 | MDK | MUC4 | CSE1L | 0.944 |
| MDK | VWF | LCN2 | 0.947 | MDK | STX7 | FCGBP | 0.944 |
| MDK | VWF | MUC4 | 0.947 | MDK | LBP | MUC4 | 0.944 |
| MDK | VWF | MUC4 | 0.947 | MDK | LCN2 | PPL | 0.944 |
| MDK | FN1 | SPRR1B | 0.947 | MDK | APOB | EPS8L1 | 0.944 |
| MDK | APOB | PPL | 0.947 | MDK | FN1 | EPS8L1 | 0.944 |
| MDK | CCT6A | ANXA7 | 0.947 | MDK | C4BPA | FCGBP | 0.944 |
| MDK | LCN2 | MDK | 0.947 | MDK | MUC4 | SERPINH1 | 0.944 |
| MDK | APOB | FCGBP | 0.947 | MDK | FN1 | VIM | 0.944 |
| MDK | APOB | FCGBP | 0.947 | MDK | FN1 | S100A9 | 0.944 |
| MDK | LCN2 | MUC4 | 0.947 | MDK | FN1 | STX7 | 0.943 |
| MDK | C1QA | MUC4 | 0.947 | MDK | TCP1 | MUC4 | 0.943 |
| MDK | SPP1 | MUC4 | 0.947 | MDK | MUC4 | HSPE1 | 0.943 |
| MDK | TCP1 | MUC4 | 0.947 | MDK | SERPIND1 | MUC4 | 0.943 |
| MDK | VWF | LCN2 | 0.946 | MDK | FN1 | CAPS | 0.943 |
| MDK | APOB | FCGBP | 0.946 | MDK | FN1 | SPP1 | 0.943 |
| MDK | APOB | SPP1 | 0.946 | MDK | FN1 | SCEL | 0.943 |
| MDK | APOB | SPP1 | 0.946 | MDK | FN1 | IMPA1 | 0.943 |
| MDK | APOB | PPL | 0.946 | MDK | APOC1 | MUC4 | 0.943 |
| MDK | FGFBP1 | PPL | 0.946 | MDK | LCN2 | LMO7 | 0.943 |
| MDK | VWF | MUC4 | 0.946 | MDK | MUC4 | CSE1L | 0.943 |
| MDK | FGFBP1 | MUC4 | 0.946 | MDK | LCN2 | MMP8 | 0.943 |
| MDK | APOF | FCGBP | 0.946 | MDK | PPL | APP | 0.943 |
| MDK | SERPIND1 | MUC4 | 0.946 | MDK | MUC4 | A2ML1 | 0.943 |
| MDK | SAA4 | MUC4 | 0.946 | MDK | SPP1 | APOF | 0.943 |
| MDK | FN1 | PPL | 0.946 | MDK | FGFBP1 | PPL | 0.943 |
| MDK | ANXA7 | CSE1L | 0.946 | MDK | FN1 | SPRR1B | 0.943 |
| MDK | FN1 | SCEL | 0.946 | MDK | LBP | LCN2 | 0.943 |
| MDK | CCT6A | MUC4 | 0.946 | MDK | MUC4 | C4BPA | 0.943 |
| MDK | ITIH2 | MUC4 | 0.946 | MDK | SPP1 | LCN2 | 0.943 |
| MDK | C1QA | MUC4 | 0.946 | MDK | C4BPA | MUC4 | 0.943 |
| MDK | APOB | LCN2 | 0.946 | MDK | FN1 | CSE1L | 0.943 |
| MDK | APOB | IMPA1 | 0.946 | MDK | FN1 | CSE1L | 0.943 |
| MDK | VWF | MUC4 | 0.946 | MDK | MUC4 | AGRN | 0.943 |
| MDK | ITIH2 | MUC4 | 0.946 | MDK | VWF | PPL | 0.943 |
| MDK | MUC4 | TCP1 | 0.946 | MDK | APOB | APP | 0.943 |
| MDK | COL1A1 | APOB | 0.946 | MDK | FN1 | SPP1 | 0.943 |
| MDK | APOB | SPP1 | 0.946 | MDK | LBP | LCN2 | 0.943 |
| MDK | LCN2 | APP | 0.946 | MDK | LCN2 | SPRR1B | 0.943 |
| MDK | LCN2 | CAMP | 0.946 | MDK | C4BPA | FCGBP | 0.943 |
| MDK | SCEL | MUC4 | 0.946 | MDK | FN1 | CD44 | 0.943 |
| MDK | VWF | FCGBP | 0.946 | MDK | MUC4 | A2ML1 | 0.943 |
| MDK | APOB | FCGBP | 0.945 | MDK | MUC4 | CALU | 0.943 |
| MDK | SPP1 | MUC4 | 0.945 | MDK | VWF | STX7 | 0.943 |
| MDK | LCN2 | MMP8 | 0.945 | MDK | APOB | APP | 0.943 |
| MDK | MUC4 | FCGBP | 0.945 | MDK | FN1 | ANXA7 | 0.943 |
| MDK | VWF | FCGBP | 0.945 | MDK | FN1 | LMNB1 | 0.943 |
| MDK | FN1 | S100A9 | 0.945 | MDK | LCAT | MUC4 | 0.943 |
| MDK | MUC4 | LCN2 | 0.945 | MDK | COL1A1 | FCGBP | 0.943 |
| MDK | ANXA7 | FCGBP | 0.945 | MDK | FN1 | APOF | 0.943 |
| MDK | FN1 | STX7 | 0.945 | MDK | APOB | CD44 | 0.943 |
| MDK | SPRR1A | MUC4 | 0.945 | MDK | MUC4 | HSPE1 | 0.943 |
| MDK | APOF | MUC4 | 0.945 | MDK | APOB | SPRR1B | 0.943 |
| MDK | APOB | STX7 | 0.945 | MDK | APOB | CD44 | 0.943 |
| MDK | SERPIND1 | MUC4 | 0.945 | MDK | VWF | LCN2 | 0.943 |
| MDK | FN1 | LMNB1 | 0.945 | MDK | SERPIND1 | PPL | 0.943 |
| MDK | SERPIND1 | FCGBP | 0.945 | MDK | FN1 | PLBD1 | 0.943 |
| MDK | SPP1 | LCN2 | 0.945 | MDK | SAA4 | FCGBP | 0.943 |
| MDK | APOF | LCN2 | 0.945 | MDK | C4BPA | MUC4 | 0.943 |
| MDK | APOB | SPP1 | 0.945 | MDK | MUC4 | APOF | 0.943 |
| MDK | FGFBP1 | MUC4 | 0.945 | MDK | MUC4 | SERPINH1 | 0.943 |
| MDK | PPL | FCGBP | 0.945 | MDK | FGFBP1 | LCN2 | 0.942 |
| MDK | MUC4 | STX7 | 0.945 | MDK | APOB | CD44 | 0.942 |
| MDK | FN1 | IMPA1 | 0.945 | MDK | FN1 | LMO7 | 0.942 |
| MDK | MUC4 | FCGBP | 0.945 | MDK | APP | LCN2 | 0.942 |
| MDK | MUC4 | CA1 | 0.945 | MDK | MUC4 | SPRR1B | 0.942 |
| MDK | SPRR1B | MUC4 | 0.945 | MDK | MUC4 | KRT8 | 0.942 |
| MDK | APOB | STX7 | 0.945 | MDK | MUC4 | CSE1L | 0.942 |
| MDK | FN1 | SPP1 | 0.945 | MDK | APP | MUC4 | 0.942 |
| MDK | FN1 | PLBD1 | 0.945 | MDK | APOB | S100A9 | 0.942 |
| MDK | SPP1 | MUC4 | 0.945 | MDK | FN1 | MMP8 | 0.942 |
| MDK | FGFBP1 | MUC4 | 0.945 | MDK | FN1 | CAMP | 0.942 |
| MDK | LCN2 | ANXA7 | 0.945 | MDK | SERPIND1 | FCGBP | 0.942 |
| MDK | MUC4 | PPL | 0.945 | MDK | FN1 | CD44 | 0.942 |
| MDK | C4BPA | MUC4 | 0.945 | MDK | MUC4 | SPRR1A | 0.942 |
| MDK | PPL | FCGBP | 0.945 | MDK | ANXA7 | APP | 0.942 |
| MDK | MUC4 | SPRR1B | 0.945 | MDK | STX7 | FCGBP | 0.942 |
| MDK | FN1 | SPP1 | 0.945 | MDK | FN1 | CCT6A | 0.942 |
| MDK | FN1 | FGFBP1 | 0.945 | MDK | FN1 | CAPS | 0.942 |
| MDK | SPP1 | LCN2 | 0.945 | MDK | FN1 | ANXA3 | 0.942 |
| MDK | LBP | MUC4 | 0.945 | MDK | SPP1 | APOF | 0.942 |

TABLE 6
2-protein biomarker detection for EC prognosis: low vs high histological grade

| Prot 1 | Prot 2 | AUC | Prot 1 | Prot 2 | AUC | Prot 1 | Prot 2 | AUC |
|---|---|---|---|---|---|---|---|---|
| PIGR | HSPE1 | 0.97 | PIGR | CTNNB1 | 0.93 | PIGR | PEA15 | 0.92 |
| PIGR | HSPE1 | 0.96 | PIGR | MDK | 0.93 | PIGR | SPP1 | 0.92 |
| PIGR | HSPE1 | 0.96 | PIGR | LDHA | 0.92 | PIGR | SPP1 | 0.92 |
| PIGR | HSPE1 | 0.95 | PIGR | VWF | 0.92 | PIGR | C1R | 0.91 |
| PIGR | AGRN | 0.95 | PIGR | ATP6V1G1 | 0.92 | PIGR | S100A9 | 0.91 |
| PIGR | CD44 | 0.95 | PIGR | PKM | 0.92 | PIGR | CAPS | 0.91 |
| PIGR | CD44 | 0.95 | PIGR | NAMPT | 0.92 | PIGR | LMNB1 | 0.91 |
| PIGR | KRT8 | 0.94 | PIGR | PEA15 | 0.92 | PIGR | PKM | 0.91 |
| PIGR | AGRN | 0.94 | PIGR | CSE1L | 0.92 | HSPE1 | FN1 | 0.91 |
| PIGR | MDK | 0.94 | PIGR | LMNB1 | 0.92 | PIGR | C4BPA | 0.91 |
| PIGR | KRT8 | 0.94 | PIGR | LMO7 | 0.92 | PIGR | CAMP | 0.91 |
| PIGR | CD44 | 0.94 | PIGR | CALU | 0.92 | PIGR | A2ML1 | 0.91 |
| PIGR | CD44 | 0.94 | PIGR | STMN1 | 0.92 | PIGR | TCP1 | 0.91 |
| PIGR | AGRN | 0.94 | PIGR | TCP1 | 0.92 | PIGR | C1R | 0.91 |
| PIGR | STMN1 | 0.94 | PIGR | LDHA | 0.92 | PIGR | LBP | 0.91 |
| PIGR | APP | 0.93 | PIGR | SPP1 | 0.92 | PIGR | ITIH2 | 0.91 |
| PIGR | AGRN | 0.93 | PIGR | VIM | 0.92 | PIGR | SPRR1B | 0.91 |
| PIGR | SERPINH1 | 0.93 | PIGR | CSE1L | 0.92 | PIGR | VIM | 0.91 |
| PIGR | CALU | 0.93 | PIGR | CCT6A | 0.92 | PIGR | NAMPT | 0.91 |
| PIGR | LGALS1 | 0.93 | PIGR | MDK | 0.92 | PIGR | CCT6A | 0.91 |
| PIGR | ATP6V1G1 | 0.93 | PIGR | SPP1 | 0.92 | VWF | HSPE1 | 0.91 |
| PIGR | CHMP4B | 0.93 | PIGR | IMPA1 | 0.92 | PIGR | APOB | 0.91 |
| PIGR | KRT8 | 0.93 | PIGR | APP | 0.92 | PIGR | ITIH2 | 0.91 |
| FN1 | HSPE1 | 0.93 | PIGR | CCT6A | 0.92 | PIGR | APP | 0.91 |
| PIGR | SERPINH1 | 0.93 | PIGR | STX7 | 0.92 | PIGR | CSE1L | 0.91 |
| PIGR | CHMP4B | 0.93 | PIGR | CALU | 0.92 | PIGR | LMO7 | 0.91 |
| PIGR | CALU | 0.93 | PIGR | LDHA | 0.92 | PIGR | C1QA | 0.91 |
| PIGR | STMN1 | 0.93 | PIGR | NEDD8-MDP1 | 0.92 | PIGR | APOF | 0.91 |
| PIGR | SERPINH1 | 0.93 | PIGR | EPS8L1 | 0.92 | PIGR | APOC1 | 0.91 |
| PIGR | CTNNB1 | 0.93 | PIGR | VIM | 0.92 | PIGR | PPL | 0.91 |
| PIGR | LGALS1 | 0.93 | PIGR | STMN1 | 0.92 | PIGR | NEDD8-MDP1 | 0.91 |
| PIGR | KRT8 | 0.93 | PIGR | LDHA | 0.92 | PIGR | C4BPA | 0.91 |
| PIGR | PKM | 0.93 | PIGR | VWF | 0.92 | PIGR | FN1 | 0.91 |
| PIGR | ANXA7 | 0.93 | PIGR | CAPS | 0.92 | PIGR | TIMP2 | 0.91 |
| PIGR | PKM | 0.93 | PIGR | VWF | 0.92 | PIGR | TMPRSS11E | 0.91 |
| PIGR | VWF | 0.93 | PIGR | LGALS1 | 0.92 | PIGR | GRN | 0.91 |
| PIGR | LGALS1 | 0.93 | PIGR | GYG1 | 0.92 | PIGR | PLBD1 | 0.91 |
| FN1 | HSPE1 | 0.93 | HSPE1 | FN1 | 0.92 | PIGR | COL3A1 | 0.91 |
| PIGR | LMNB1 | 0.93 | PIGR | SPRR1A | 0.92 | PIGR | COL1A1 | 0.91 |
| PIGR | RAB21 | 0.93 | PIGR | PPL | 0.92 | PIGR | C4BPA | 0.91 |
| PIGR | SERPINH1 | 0.93 | PIGR | RAB21 | 0.92 | PIGR | FN1 | 0.91 |

Prot.: Protein, AUC: Area Under the Curve

TABLE 7
3-protein biomarker detection for EC prognosis: low vs high histological grade

| Prot 1 | Prot 2 | Prot 3 | AUC | Prot 1 | Prot 2 | Prot 3 | AUC |
|---|---|---|---|---|---|---|---|
| PIGR | HSPE1 | EPS8L1 | 0.975 | PIGR | HSPE1 | CA1 | 0.965 |
| PIGR | VWF | HSPE1 | 0.975 | PIGR | HSPE1 | STMN1 | 0.965 |
| PIGR | C1QA | HSPE1 | 0.974 | PIGR | HSPE1 | STMN1 | 0.965 |
| PIGR | HSPE1 | COL3A1 | 0.974 | PIGR | HSPE1 | ANXA7 | 0.965 |
| PIGR | HSPE1 | ANXA7 | 0.973 | PIGR | HSPE1 | C1QA | 0.964 |
| PIGR | HSPE1 | COL3A1 | 0.971 | PIGR | HSPE1 | C1R | 0.964 |
| PIGR | HSPE1 | SCEL | 0.971 | PIGR | HSPE1 | NEDD8-MDP1 | 0.964 |
| PIGR | HSPE1 | LMO7 | 0.971 | PIGR | HSPE1 | COL1A1 | 0.964 |
| PIGR | HSPE1 | PKM | 0.971 | PIGR | VWF | CD44 | 0.964 |
| PIGR | VWF | HSPE1 | 0.971 | PIGR | HSPE1 | C1R | 0.964 |
| PIGR | HSPE1 | CSE1L | 0.971 | PIGR | HSPE1 | MUC4 | 0.964 |
| PIGR | HSPE1 | TCP1 | 0.970 | PIGR | HSPE1 | ANXA3 | 0.964 |
| PIGR | HSPE1 | SERPIND1 | 0.970 | PIGR | HSPE1 | LMO7 | 0.964 |
| PIGR | HSPE1 | CSE1L | 0.970 | PIGR | HSPE1 | TCP1 | 0.964 |
| PIGR | HSPE1 | CALU | 0.970 | PIGR | HSPE1 | HCLS1 | 0.964 |
| PIGR | HSPE1 | PEA15 | 0.970 | PIGR | HSPE1 | SERPINH1 | 0.963 |
| PIGR | HSPE1 | GYG1 | 0.970 | PIGR | HSPE1 | TCP1 | 0.963 |
| PIGR | HSPE1 | SPP1 | 0.970 | PIGR | HSPE1 | CA1 | 0.963 |
| PIGR | HSPE1 | CTNNB1 | 0.969 | PIGR | HSPE1 | COL1A1 | 0.963 |
| PIGR | C1QA | HSPE1 | 0.969 | PIGR | HSPE1 | GYG1 | 0.963 |
| PIGR | HSPE1 | COL3A1 | 0.968 | PIGR | HSPE1 | CALU | 0.962 |
| PIGR | HSPE1 | ANXA3 | 0.968 | PIGR | HSPE1 | NAMPT | 0.962 |
| PIGR | HSPE1 | IGFALS | 0.968 | PIGR | HSPE1 | CCT6A | 0.962 |
| PIGR | HSPE1 | COL1A1 | 0.968 | PIGR | HSPE1 | VIM | 0.962 |
| PIGR | HSPE1 | HCLS1 | 0.968 | PIGR | HSPE1 | MUC4 | 0.961 |
| PIGR | HSPE1 | TCP1 | 0.968 | PIGR | HSPE1 | FGFBP1 | 0.961 |
| PIGR | C4BPA | HSPE1 | 0.968 | PIGR | HSPE1 | EPS8L1 | 0.961 |
| PIGR | HSPE1 | LDHA | 0.968 | PIGR | HSPE1 | PEA15 | 0.961 |
| PIGR | HSPE1 | CA1 | 0.968 | PIGR | HSPE1 | PKM | 0.961 |
| PIGR | HSPE1 | RAB21 | 0.967 | PIGR | HSPE1 | SERPINH1 | 0.961 |
| PIGR | HSPE1 | TCP1 | 0.966 | PIGR | HSPE1 | SERPIND1 | 0.961 |
| PIGR | HSPE1 | LDHA | 0.966 | PIGR | HSPE1 | PEA15 | 0.961 |
| PIGR | HSPE1 | MMP8 | 0.966 | PIGR | HSPE1 | CD44 | 0.961 |
| PIGR | HSPE1 | ATP6V1G1 | 0.966 | PIGR | HSPE1 | APP | 0.961 |
| PIGR | HSPE1 | PKM | 0.966 | PIGR | CD44 | AGRN | 0.961 |
| PIGR | HSPE1 | LSP1 | 0.966 | PIGR | CD44 | SPP1 | 0.961 |
| PIGR | HSPE1 | EPS8L1 | 0.966 | PIGR | HSPE1 | CSE1L | 0.961 |
| PIGR | HSPE1 | HSPE1 | 0.965 | PIGR | HSPE1 | GRN | 0.961 |

Prot.: Protein, AUC: Area Under the Curve

TABLE 8
2-protein biomarker detection for EC diagnosis and prognosis, including the differentiation between
histological grades; and subtypes of ECs (i.e., Endometrioid, Serous, Others (carcinosarcoma).

| Prot 1 | Prot 2 | AUC | Prot 1 | Prot 2 | AUC |
|---|---|---|---|---|---|
| PIGR_ILLN *** | RAB21_GIEE ** | 0.89 | PIGR_ILLN *** | ANXA7_SEID * | 0.86 |
| PIGR_ILLN *** | PKM_NTGI ** | 0.89 | PIGR_ILLN *** | PEA15_ISEE. | 0.86 |
| PIGR_ILLN *** | LDHA_VTLT ** | 0.89 | PIGR_ILLN *** | TCP1_SSLG. | 0.86 |
| PIGR_ILLN *** | LDHA_LVII ** | 0.89 | PIGR_ILLN *** | HSPE1_VLLP. | 0.86 |
| PIGR_VYTV *** | LDHA_VTLT ** | 0.89 | PIGR_ILLN *** | CHMP4B_GGPT. | 0.86 |
| PIGR_VYTV *** | LDHA_LVII ** | 0.89 | PIGR_ILLN *** | CCT6A_GIDP. | 0.86 |
| PIGR_VYTV *** | RAB21_GIEE ** | 0.89 | PIGR_ILLN *** | S100A9_LGHP | 0.86 |
| PIGR_ILLN *** | GYG1_GALV * | 0.88 | PIGR_VYTV *** | CCT6A_VATA. | 0.86 |
| PIGR_VYTV *** | PKM_NTGI ** | 0.88 | SERPINH1_GVVE *** | CAPS_EAVI ** | 0.86 |
| PIGR_ILLN *** | SERPINH1_DTQS ** | 0.88 | PIGR_ILLN *** | SPRR1A_VPEP | 0.86 |
| PIGR_ILLN *** | NAMPT_YLLE ** | 0.88 | PIGR_ILLN *** | STX7_ITQC * | 0.86 |
| SERPINH1_DTQS *** | CAPS_SGDG *** | 0.88 | PIGR_ILLN *** | STMN1_AIEE * | 0.86 |
| PIGR_ILLN *** | CSE1L_SANV ** | 0.88 | PIGR_ILLN *** | STMN1_SHEA * | 0.86 |
| PIGR_VYTV *** | SERPINH1_DTQS ** | 0.88 | PIGR_ILLN *** | CALU_HLVY * | 0.86 |
| PIGR_ILLN *** | SERPINH1_GVVE ** | 0.88 | PIGR_VYTV *** | CCT6A_GIDP. | 0.86 |
| PIGR_ILLN *** | CSE1L_LLQT ** | 0.88 | PIGR_ILLN *** | LMNB1_AEHD * | 0.86 |
| PIGR_VYTV *** | PKM_APII * | 0.88 | PIGR_ILLN *** | CTNNB1_LVQL * | 0.86 |
| PIGR_ILLN *** | PKM_APII. | 0.88 | PIGR_ILLN *** | APP_VESL | 0.86 |
| PIGR_ILLN *** | IMPA1_YPSH ** | 0.88 | PIGR_VYTV *** | TCP1_SSLG. | 0.86 |
| PIGR_VYTV *** | NAMPT_YLLE ** | 0.88 | PIGR_VYTV *** | ITGB2_ALNE ** | 0.86 |
| PIGR_ILLN *** | LBP_LAEG * | 0.87 | PIGR_VYTV *** | HSPE1_VLLP | 0.86 |
| SERPINH1_GVVE *** | CAPS_SGDG *** | 0.87 | PIGR_VYTV *** | ANXA7_SEID * | 0.86 |
| PIGR_ILLN *** | CD44_ALSI * | 0.87 | PIGR_VYTV *** | CALU_HLVY * | 0.86 |
| PIGR_VYTV *** | SERPINH1_GVVE ** | 0.87 | PIGR_VYTV *** | LGALS1_SFVL * | 0.86 |
| PIGR_VYTV *** | CSE1L_SANV ** | 0.87 | PIGR_VYTV *** | LMNB1_IGDT * | 0.86 |
| PIGR_VYTV *** | GYG1_GALV * | 0.87 | PIGR_ILLN *** | MUC4_SLEP | 0.86 |
| PIGR_VYTV *** | IMPA1_YPSH * | 0.87 | PIGR_ILLN *** | COL12A1_LNWN | 0.86 |
| PIGR_VYTV *** | CSE1L_LLQT ** | 0.87 | PIGR_ILLN *** | CALU_TFDQ. | 0.86 |
| PIGR_ILLN *** | LGALS1_LPDG ** | 0.87 | PIGR_VYTV *** | CTNNB1_LVQL * | 0.86 |
| PIGR_VYTV *** | LBP_LAEG * | 0.87 | PIGR_VYTV *** | CHMP4B_GGPT. | 0.86 |
| PIGR_ILLN *** | AGRN_VLGA ** | 0.87 | PIGR_VYTV *** | MMP8_YYAF ** | 0.86 |
| PIGR_VYTV *** | CD44_ALSI * | 0.87 | PIGR_ILLN *** | HCLS1_SAVG * | 0.86 |
| PIGR_ILLN *** | ITGB2_ALNE ** | 0.87 | PIGR_VYTV *** | S100A9_LGHP | 0.86 |
| PIGR_ILLN *** | CD44_YGFI * | 0.87 | PIGR_ILLN *** | CAPS_SGDG | 0.86 |
| PIGR_ILLN *** | MMP8_YYAF ** | 0.87 | PIGR_ILLN *** | ITIH2_FYNQ | 0.86 |
| PIGR_VYTV *** | ATP6V1G1_EEAQ * | 0.87 | PIGR_VYTV *** | PEA15_ISEE | 0.86 |
| PIGR_ILLN *** | HSPE1_FLPL * | 0.87 | PIGR_ILLN *** | TMPRSS11E_MLCA | 0.86 |
| PIGR_VYTV *** | LGALS1_LPDG ** | 0.87 | PIGR_ILLN *** | TCP1_GAND. | 0.86 |
| SERPINH1_DTQS *** | CAPS_EAVI ** | 0.87 | PIGR_VYTV *** | NEDD8-MDP1_EIEI * | 0.86 |
| PIGR_ILLN *** | LGALS1_SFVL ** | 0.87 | PIGR_VYTV *** | STMN1_AIEE * | 0.86 |
| PIGR_ILLN *** | LSP1_EDSD * | 0.87 | PIGR_ILLN *** | SPRR1B_VPEP | 0.86 |
| PIGR_VYTV *** | CD44_YGFI * | 0.87 | PIGR_VYTV *** | VIM_ISLP * | 0.86 |
| PIGR_ILLN *** | ATP6V1G1_EEAQ * | 0.87 | PIGR_ILLN *** | VIM_ISLP * | 0.86 |
| PIGR_VYTV *** | AGRN_VLGA ** | 0.87 | PIGR_ILLN *** | ANXA3_DYPD * | 0.86 |
| PIGR_ILLN *** | LMNB1_IGDT * | 0.87 | PIGR_VYTV *** | STMN1_SHEA * | 0.86 |
| PIGR_ILLN *** | NEDD8-MDP1_EIEI * | 0.87 | PIGR_VYTV *** | CALU_TFDQ. | 0.86 |
| PIGR_ILLN *** | CCT6A_VATA. | 0.86 | PIGR_VYTV *** | APP_VESL | 0.86 |
| PIGR_VYTV *** | HSPE1_FLPL * | 0.86 | PIGR_ILLN *** | TMPRSS11E_TDAC | 0.86 |
| PIGR_VYTV *** | LSP1_EDSD. | 0.86 | PIGR_ILLN *** | VIM_ILLA * | 0.86 |

| Prot 1 | Prot 2 | AUC |
|---|---|---|
| PIGR_ILLN *** | AGRN_EAAC. | 0.855534 |
| PIGR_ILLN *** | EPS8L1_APEP | 0.8551456 |
| PIGR_VYTV *** | LMNB1_AEHD * | 0.8551456 |
| PIGR_ILLN *** | VWF_VIVI | 0.8547573 |
| PIGR_ILLN *** | ITIH2_IQPS | 0.8547573 |
| PIGR_ILLN *** | VWF_YAGS | 0.8543689 |
| PIGR_ILLN *** | C4BPA_LSCS | 0.8543689 |
| PIGR_VYTV *** | TMPRSS11E_MLCA | 0.8543689 |
| PIGR_ILLN *** | GRN_QHCC | 0.8539806 |
| PIGR_ILLN *** | SPP1_DSYE * | 0.8539806 |
| PIGR_VYTV *** | AGRN_EAAC * | 0.8539806 |
| PIGR_ILLN *** | PLBD1_LGLD | 0.8535922 |
| PIGR_ILLN *** | KRT8_WSLL | 0.8535922 |
| PIGR_VYTV *** | SPRR1A_VPEP | 0.8535922 |
| PIGR_VYTV *** | HCLS1_SAVG. | 0.8535922 |
| PIGR_VYTV *** | TCP1_GAND. | 0.8532039 |
| PIGR_VYTV *** | STX7_ITQC. | 0.8532039 |
| PIGR_ILLN *** | LCN2_VPLQ. | 0.8528155 |
| PIGR_ILLN *** | C1QA_SLGF | 0.8528155 |
| PIGR_ILLN *** | PPL_VVLQ | 0.8528155 |
| PIGR_ILLN *** | SPRR1B_QPCT | 0.8528155 |
| PIGR_ILLN *** | CAPS_EAVI | 0.8528155 |
| PIGR_ILLN *** | FAM107B_LEQL | 0.8528155 |
| PIGR_VYTV *** | MUC4_SLEP | 0.8528155 |
| PIGR_ILLN *** | FCGBP_ISVA. | 0.8524272 |
| PIGR_ILLN *** | C1R_ESEQ | 0.8524272 |
| PIGR_VYTV *** | COL12A1_LNWN | 0.8524272 |
| PIGR_ILLN *** | MUC4_LECL | 0.8520388 |
| PIGR_ILLN *** | SERPINA3_NLAV | 0.8520388 |
| PIGR_ILLN *** | APOB_FVTQ | 0.8516505 |
| PIGR_VYTV *** | ITIH2_FYNQ | 0.8516505 |
| PIGR_VYTV *** | CAPS_SGDG | 0.8516505 |
| PIGR_ILLN *** | APOF_SGVQ | 0.8512621 |
| PIGR_ILLN *** | LCN2_ELTS. | 0.8512621 |
| PIGR_ILLN *** | C4BPA_FSAI | 0.8512621 |
| PIGR_ILLN *** | FGFBP1_LVSS | 0.8512621 |
| PIGR_VYTV *** | VWF_VIVI | 0.8512621 |
| PIGR_ILLN | PIGR_VYTV | 0.8508738 |
| PIGR_ILLN *** | APOF_SLPT | 0.8508738 |
| PIGR_ILLN *** | SERPIND1_NYNL | 0.8508738 |
| PIGR_VYTV *** | SPRR1B_VPEP | 0.8508738 |
| PIGR_VYTV *** | KRT8_WSLL | 0.8508738 |
| PIGR_ILLN *** | FCGBP_AGCV. | 0.8504854 |
| PIGR_ILLN *** | FN1_WCGT | 0.8504854 |
| PIGR_ILLN *** | FGFBP1_DTLG | 0.8504854 |
| PIGR_VYTV *** | KRT8_LSEL | 0.8504854 |
| PIGR_ILLN *** | SERPIND1_SVND | 0.8500971 |
| PIGR_ILLN *** | APOB_LLSG | 0.8500971 |
| PIGR_ILLN *** | IGFALS_LWLE | 0.8500971 |
| PIGR_ILLN *** | KRT8_LSEL | 0.8500971 |
| PIGR_VYTV *** | FCGBP_ISVA. | 0.8500971 |

Prot.: Protein,
AUC: Area Under the Curve

TABLE 9
3- protein biomarker detection for EC diagnosis and prognosis, including
the differentiation between histological grades; and subtypes of
ECs (i.e., Endometrioid, Serous, Others (carcinosarcoma).

| Prot 1 | Prot 2 | Prot 3 | AUC |
|---|---|---|---|
| PIGR_ILLN *** | PKM_APII ** | MMP8_YYAF *** | 0.933 |
| PIGR_VYTV *** | PKM_APII ** | MMP8_YYAF *** | 0.929 |
| PIGR_ILLN ** | SERPINH1_DTQS *** | CAPS_SGDG ** | 0.918 |
| PIGR_VYTV ** | SERPINH1_DTQS *** | CAPS_SGDG ** | 0.913 |
| PIGR_ILLN *** | LMO7_EQVP * | PKM_NTGI ** | 0.912 |
| PIGR_ILLN *** | LBP_LAEG. | PKM_NTGI ** | 0.911 |
| PIGR_VYTV *** | LBP_LAEG * | PKM_NTGI ** | 0.911 |
| PIGR_ILLN *** | RAB21_GIEE *** | CAPS_SGDG. | 0.910 |
| PIGR_ILLN *** | PKM_NTGI *** | EPS8L1_APEP * | 0.910 |
| PIGR_ILLN *** | CHMP4B_GGPT ** | MMP8_YYAF *** | 0.910 |
| PIGR_ILLN *** | ATP6V1G1_EEAQ ** | MMP8_YYAF *** | 0.910 |
| PIGR_ILLN ** | SERPINH1_GVVE *** | CAPS_SGDG * | 0.909 |
| PIGR_VYTV *** | LMO7_EQVP ** | PKM_NTGI ** | 0.909 |
| PIGR_ILLN *** | PKM_APII ** | CAMP_AIDG *** | 0.908 |
| PIGR_ILLN *** | PKM_APII *** | LCN2_VPLQ *** | 0.908 |
| PIGR_ILLN *** | PKM_NTGI ** | CAPS_SGDG. | 0.908 |
| PIGR_ILLN *** | PKM_APII ** | ITGB2_ALNE *** | 0.907 |
| PIGR_ILLN *** | PKM_NTGI * | MMP8_YYAF * | 0.907 |
| PIGR_ILLN *** | LBP_LAEG. | NAMPT_YLLE ** | 0.906 |
| PIGR_VYTV *** | PKM_APII *** | CAMP_AIDG *** | 0.906 |
| PIGR_VYTV *** | PKM_NTGI *** | EPS8L1_APEP * | 0.906 |
| PIGR_ILLN *** | PKM_APII *** | LCN2_ELTS *** | 0.906 |
| PIGR_ILLN *** | CSE1L_SANV ** | CAPS_SGDG * | 0.906 |
| PIGR_ILLN *** | LBP_LAEG * | CD44_ALSI * | 0.906 |
| PIGR_ILLN *** | LDHA_LVII ** | CAPS_SGDG. | 0.906 |
| PIGR_VYTV ** | SERPINH1_GVVE *** | CAPS_SGDG * | 0.905 |
| PIGR_ILLN ** | SERPINH1_DTQS *** | CAPS_EAVI * | 0.905 |
| SERPINH1_DTQS *** | PKM_APII * | CAPS_SGDG *** | 0.905 |
| PIGR_ILLN *** | LDHA_LVII ** | ITIH2_FYNQ. | 0.904 |
| PIGR_ILLN *** | LDHA_LVII *** | APP_VESL * | 0.904 |
| SERPINH1_DTQS *** | LBP_LAEG * | CAPS_SGDG ** | 0.904 |
| PIGR_ILLN * | PIGR_VYTV. | RAB21_GIEE *** | 0.904 |
| PIGR_ILLN *** | LDHA_VTLT ** | ITIH2_FYNQ. | 0.904 |
| PIGR_ILLN *** | NAMPT_YLLE *** | LMO7_EQVP * | 0.904 |
| PIGR_ILLN *** | VWF_YAGS | PKM_NTGI ** | 0.904 |
| PIGR_ILLN *** | LDHA_VTLT ** | APP_VESL * | 0.904 |
| PIGR_VYTV ** | SERPINH1_DTQS *** | CAPS_EAVI * | 0.904 |
| PIGR_ILLN *** | LBP_LAEG | LDHA_VTLT * | 0.903 |
| PIGR_ILLN *** | LDHA_VTLT | RAB21_GIEE. | 0.903 |
| PIGR_ILLN *** | LDHA_VTLT ** | COL12A1_LNWN | 0.903 |
| PIGR_VYTV *** | PKM_APII *** | LCN2_ELTS *** | 0.903 |

| Prot 1 | Prot 2 | Prot 3 | |
|---|---|---|---|
| PIGR_ILLN *** | LDHA_LVII | RAB21_GIEE | 0.903 |
| PIGR_VYTV *** | LBP_LAEG | LDHA_LVII * | 0.903 |
| PIGR_VYTV *** | NAMPT_YLLE ** | LMO7_EQVP * | 0.903 |
| PIGR_ILLN *** | LBP_LAEG | LDHA_LVII ** | 0.903 |
| PIGR_ILLN *** | VWF_VIVI | PKM_NTGI ** | 0.903 |
| PIGR_ILLN *** | LDHA_VTLT ** | S100A9_LGHP | 0.903 |
| PIGR_ILLN *** | LDHA_LVII. | NAMPT_YLLE | 0.903 |
| PIGR_ILLN *** | LDHA_LVII ** | ITIH2_IQPS. | 0.903 |
| PIGR_ILLN *** | PKM_NTGI ** | COL3A1_FTYT. | 0.903 |
| PIGR_ILLN *** | LBP_LAEG | RAB21_GIEE ** | 0.902 |
| PIGR_ILLN *** | LBP_LAEG * | ATP6V1G1_EEAQ * | 0.902 |
| PIGR_ILLN *** | LDHA_VTLT ** | ITIH2_IQPS. | 0.902 |
| PIGR_ILLN *** | LDHA_VTLT ** | CAPS_SGDG | 0.902 |
| PIGR_ILLN *** | LDHA_LVII ** | ANXA7_SEID | 0.902 |
| PIGR_VYTV *** | PKM_APII ** | ITGB2_ALNE *** | 0.902 |
| PIGR_VYTV *** | CSE1L_SANV ** | CAPS_SGDG * | 0.902 |
| PIGR_VYTV *** | LBP_LAEG | LDHA_VTLT * | 0.902 |
| PIGR_ILLN *** | TMPRSS11E_MLCA | LDHA_LVII ** | 0.901 |
| PIGR_ILLN *** | LDHA_VTLT * | IMPA1_YPSH | 0.901 |
| PIGR_VYTV *** | LDHA_LVII ** | CAPS_SGDG | 0.901 |
| PIGR_ILLN *** | LDHA_VTLT. | NAMPT_YLLE | 0.901 |
| PIGR_ILLN *** | LDHA_VTLT * | ANXA7_SEID | 0.901 |
| PIGR_ILLN *** | LDHA_LVII ** | CAPS_EAVI | 0.901 |
| PIGR_ILLN *** | RAB21_GIEE *** | CAPS_EAVI | 0.901 |
| PIGR_ILLN *** | PKM_NTGI ** | PEA15_ISEE | 0.901 |
| PIGR_ILLN *** | PKM_APII. | RAB21_GIEE ** | 0.901 |
| PIGR_ILLN *** | CSE1L_LLQT ** | CAPS_SGDG * | 0.901 |
| PIGR_ILLN *** | LBP_LAEG * | CD44_YGFI * | 0.901 |
| PIGR_ILLN *** | LDHA_LVII | PKM_NTGI | 0.901 |
| PIGR_ILLN *** | LDHA_LVII ** | SPP1_ANDE | 0.901 |
| PIGR_ILLN *** | PKM_NTGI ** | CAPS_EAVI | 0.901 |
| PIGR_VYTV *** | PKM_NTGI ** | COL3A1_FTYT * | 0.901 |
| PIGR_ILLN ** | SERPINH1_GVVE *** | CAPS_EAVI * | 0.900 |
| PIGR_ILLN *** | LDHA_LVII ** | S100A9_LGHP | 0.900 |
| PIGR_ILLN *** | LDHA_LVII ** | SPP1_DSYE | 0.900 |
| PIGR_ILLN *** | LDHA_LVII * | IMPA1_YPSH | 0.900 |
| PIGR_ILLN *** | LDHA_LVII ** | COL12A1_LNWN | 0.900 |
| PIGR_ILLN *** | PKM_NTGI ** | PPL_VVLQ | 0.900 |
| PIGR_VYTV *** | PKM_APII *** | LCN2_VPLQ ** | 0.900 |
| PIGR_VYTV *** | LDHA_LVII ** | S100A9_LGHP | 0.900 |

Prot.: Protein,
AUC: Area Under the Curve

### Example 2

### Markers Indicating Recurrence of Disease

The discovery study included 20 patients, 9 from which turned to suffer recurrence after 5-years follow-up. These recurrent women, recur between 10 and 119 months after surgery. Specifically, there were 4 endometrioid EC (2 G2 and 2 G3) and 5 non-endometrioid EC recurring, while 10 EEC (2 G1, 7 G2, 1 G3) and 1 non-EEC did not recur. Hence, it was compared the 9 recurrent vs 11 non-recurrent patients.

Statistical analysis showed 19 proteins with AUC>0.75 to differentiate between recurrent and non-recurrent EC patients (MUC1, PRSS8, PNP, APEH, MUC16, C9, SERPINC1, SERPINA1, F2, AMBP, HP, SERPINA3, CFB, ORM2, CAT, GNAI2, A1BG, FN1 and C7). Additionally, by presence-absence statistical analysis, there were identified 18 differential expressed proteins between recurrent and non-recurrent EC patients (ASTRGL1, B4GALT1, CAPS, CBX3, CD163, CDV3, DMBT1, DSG3, EHD1, GOLM1, MUC16, MUC5AC, NME1, NT5E, PDLIM5, PRSS8, RDX, and VASP).

Next Table 10 (A) and (B) illustrate the data:

(A) Statistical analysis

| Gene names | Fold change | AUC |
|---|---|---|
| MUC1 | 1.696 | 0.889 |
| PRSS8 | 1.613 | 0.889 |
| PNP | 0.975 | 0.843 |
| APEH | 0.704 | 0.778 |
| MUC16 | 1.618 | 0.778 |
| C9 | −1.398 | 0.815 |
| SERPINC1 | −0.980 | 0.88 |
| SERPINA1 | 1.446 | 0.815 |
| F2 | −1.022 | 0.806 |
| AMBP | −0.895 | 0.787 |
| HP | −1.051 | 0.792 |
| SERPINA3 | −1.883 | 0.815 |
| CFB | −0.563 | 0.778 |
| ORM2 | −0.713 | 0.759 |
| CAT | 0.687 | 0.769 |
| GNAI2 | 0.603 | 0.769 |
| A1BG | −0.626 | 0.787 |
| FN1 | −0.854 | 0.778 |
| C7 | −0.564 | 0.778 |

(B)Statistical analysis

| | No recurrent | Recurrent | |
|---|---|---|---|
| Gene names | identifications | identifications | significance |
| ADRGL1 | 7 | 2 | 0.07 |
| B4GALT1 | 8 | 2 | 0.03 |
| CAPS | 9 | 3 | 0.04 |
| CBX3 | 11 | 6 | 0.07 |
| CD163 | 4 | 9 | 0.00 |
| CDV3 | 9 | 3 | 0.04 |
| DMBT1 | 9 | 3 | 0.04 |
| DSG3 | 9 | 4 | 0.09 |
| EHD1 | 8 | 3 | 0.08 |
| GOLM1 | 9 | 3 | 0.04 |
| MUC16 | 10 | 5 | 0.09 |
| MUC5AC | 11 | 5 | 0.03 |
| NME1 | 11 | 5 | 0.03 |
| NT5E | 9 | 3 | 0.04 |
| PDLIM5 | 10 | 5 | 0.09 |
| PRSS8 | 11 | 4 | 0.01 |
| RDX | 8 | 2 | 0.03 |
| VASP | 11 | 6 | 0.07 |

Next Table 11 lists the names of the used biomarkers.

| Gene Names | Entry | Entry Name | Protein names |
|---|---|---|---|
| MUC1 PUM | P15941 | MUC1_HUMAN | Mucin-1, MUC-1 (Breast carcinoma- |
| | | | associated antigen DF3) (Cancer |
| | | | antigen 15-3, CA 15-3) (Carcinoma- |
| | | | associated mucin) (Episialin) |
| | | | (H23AG) (Krebs von den Lungen-6, |
| | | | KL-6) (PEMT) (Peanut-reactive |
| | | | urinary mucin, PUM) (Polymorphic |
| | | | epithelial mucin, PEM) (Tumor- |
| | | | associated epithelial membrane |
| | | | antigen, EMA) (Tumor-associated |
| | | | mucin) (CD antigen CD227) [Cleaved |
| | | | into: Mucin-1 subunit alpha, MUC1- |
| | | | NT, MUC1-alpha; Mucin-1 subunit |
| | | | beta, MUC1-beta (MUC1-CT)] |
| PRSS8 | Q16651 | PRSS8_HUMAN | Prostasin, EC 3.4.21.- (Channel- |
| | | | activating protease 1, CAP1) (Serine |
| | | | protease 8) [Cleaved into: Prostasin |
| | | | light chain; Prostasin heavy chain] |
| PNP NP | P00491 | PNPH_HUMAN | Purine nucleoside phosphorylase, |
| | | | PNP, EC 2.4.2.1 (Inosine phosphorylase) |
| | | | (Inosine-guanosine phosphorylase) |
| APEH D3F15S2 | P13798 | ACPH_HUMAN | Acylamino-acid-releasing enzyme, |
| D3S48E DNF15S2 | | | AARE, EC 3.4.19.1 (Acyl-peptide |
| | | | hydrolase, APH) (Acylaminoacyl- |
| | | | peptidase) (Oxidized protein |
| | | | hydrolase, OPH) |
| MUC16 CA125 | Q8WXI7 | MUC16_HUMAN | Mucin-16, MUC-16 (Ovarian cancer- |
| | | | related tumor marker CA125, CA- |
| | | | 125) (Ovarian carcinoma antigen |
| | | | CA125) |
| C9 | P02748 | CO9_HUMAN | Complement component C9 [Cleaved |
| | | | into: Complement component C9a; |
| | | | Complement component C9b] |
| SERPINC1 AT3 | P01008 | ANT3_HUMAN | Antithrombin-III, ATIII (Serpin C1) |
| PRO0309 | | | |
| SERPINA1 AAT PI | P01009 | A1AT_HUMAN | Alpha-1-antitrypsin (Alpha-1 protease |
| PRO0684 | | | inhibitor) (Alpha-1-antiproteinase) |
| PRO2209 | | | (Serpin A1) [Cleaved into: Short |
| | | | peptide from AAT, SPAAT] |
| F2 | P00734 | THRB_HUMAN | Prothrombin, EC 3.4.21.5 |
| | | | (Coagulation factor II) [Cleaved into: |
| | | | Activation peptide fragment 1; |
| | | | Activation peptide fragment 2; |
| | | | Thrombin light chain; Thrombin heavy |
| | | | chain] |
| AMBP HCP ITIL | P02760 | AMBP_HUMAN | Protein AMBP (Protein HC) [Cleaved |
| | | | into: Alpha-1-microglobulin, EC |
| | | | 1.6.2.- (Alpha-1 microglycoprotein) |
| | | | (Complex-forming glycoprotein |
| | | | heterogeneous in charge); Inter- |
| | | | alpha-trypsin inhibitor light chain, ITI- |
| | | | LC (Bikunin) (EDC1) (HI-30) (Uronic- |
| | | | acid-rich protein); Trypstatin] |
| HP | P00738 | HPT_HUMAN | Haptoglobin (Zonulin) [Cleaved into: |
| | | | Haptoglobin alpha chain; Haptoglobin |
| | | | beta chain] |
| SERPINA3 AACT | P01011 | AACT_HUMAN | Alpha-1-antichymotrypsin, ACT (Cell |
| GIG24 GIG25 | | | growth-inhibiting gene 24/25 protein) |
| | | | (Serpin A3) [Cleaved into: Alpha-1- |
| | | | antichymotrypsin His-Pro-less] |
| CFB BF BFD | P00751 | CFAB_HUMAN | Complement factor B, EC 3.4.21.47 |
| | | | (C3/C5 convertase) (Glycine-rich beta |
| | | | glycoprotein, GBG) (PBF2) |
| | | | (Properdin factor B) [Cleaved into: |
| | | | Complement factor B Ba fragment; |
| | | | Complement factor B Bb fragment] |
| ORM2 AGP2 | P19652 | A1AG2_HUMAN | Alpha-1-acid glycoprotein 2, AGP 2 |
| | | | (Orosomucoid-2, OMD 2) |
| CAT | P04040 | CATA_HUMAN | Catalase, EC 1.11.1.6 |
| GNAI2 GNAI2B | P04899 | GNAI2_HUMAN | Guanine nucleotide-binding protein |
| | | | G(i) subunit alpha-2 (Adenylate |
| | | | cyclase-inhibiting G alpha protein) |
| A1BG | P04217 | A1BG_HUMAN | Alpha-1B-glycoprotein (Alpha-1-B |
| | | | glycoprotein) |
| FN1 FN | P02751 | FINC_HUMAN | Fibronectin, FN (Cold-insoluble |
| | | | globulin, CIG) [Cleaved into: |
| | | | Anastellin; Ugl-Y1; Ugl-Y2; Ugl-Y3] |
| C7 | P10643 | CO7_HUMAN | Complement component C7 |
| ASRGL1 ALP | Q7L266 | ASGL1_HUMAN | Isoaspartyl peptidase/L- |
| CRASH | | | asparaginase, EC 3.4.19.5, EC |
| | | | 3.5.1.1 (Asparaginase-like protein 1) |
| | | | (Beta-aspartyl-peptidase) (Isoaspartyl |
| | | | dipeptidase) (L-asparagine |
| | | | amidohydrolase) [Cleaved into: |
| | | | Isoaspartyl peptidase/L-asparaginase |
| | | | alpha chain; Isoaspartyl peptidase/L- |
| | | | asparaginase beta chain] |
| B4GALT1 GGTB2 | P15291 | B4GT1_HUMAN | Beta-1,4-galactosyltransferase 1, |
| | | | Beta-1,4-GalTase 1, Beta4Gal-T1, |
| | | | b4Gal-T1, EC 2.4.1.- (Beta-N- |
| | | | acetylglucosaminyl-glycolipid beta- |
| | | | 1,4-galactosyltransferase) (Beta-N- |
| | | | acetylglucosaminylglycopeptide beta- |
| | | | 1,4-galactosyltransferase, EC |
| | | | 2.4.1.38) (Lactose synthase A |
| | | | protein, EC 2.4.1.22) (N- |
| | | | acetyllactosamine synthase, EC |
| | | | 2.4.1.90) (Nal synthase) |
| | | | (Neolactotriaosylceramide beta-1,4- |
| | | | galactosyltransferase, EC 2.4.1.275) |
| | | | (UDP-Gal:beta-GlcNAc beta-1,4- |
| | | | galactosyltransferase 1) (UDP- |
| | | | galactose:beta-N-acetylglucosamine |
| | | | beta-1,4-galactosyltransferase 1) |
| | | | [Cleaved into: Processed beta-1,4- |
| | | | galactosyltransferase 1] |
| CAPS | Q13938 | CAYP1_HUMAN | Calcyphosin (Calcyphosine) |
| CBX3 | Q13185 | CBX3_HUMAN | Chromobox protein homolog 3 |
| | | | (HECH) (Heterochromatin protein 1 |
| | | | homolog gamma, HP1 gamma) |
| | | | (Modifier 2 protein) |
| CD163 M130 | Q86VB7 | C163A_HUMAN | Scavenger receptor cysteine-rich type |
| | | | 1 protein M130 (Hemoglobin |
| | | | scavenger receptor) (CD antigen |
| | | | CD163) [Cleaved into: Soluble |
| | | | CD163, sCD163] |
| CDV3 H41 | Q9UKY7 | CDV3_HUMAN | Protein CDV3 homolog |
| DMBT1 GP340 | Q9UGM3 | DMBT1_HUMAN | Deleted in malignant brain tumors 1 |
| | | | protein (Glycoprotein 340, Gp-340) |
| | | | (Hensin) (Salivary agglutinin, SAG) |
| | | | (Surfactant pulmonary-associated D- |
| | | | binding protein) |
| DSG3 CDHF6 | P32926 | DSG3_HUMAN | Desmoglein-3 (130 kDa pemphigus |
| | | | vulgaris antigen, PVA) (Cadherin |
| | | | family member 6) |
| EHD1 PAST | Q9H4M9 | EHD1_HUMAN | EH domain-containing protein 1 |
| PAST1 | | | (PAST homolog 1, hPAST1) (Testilin) |
| CDABP0131 | | | |
| GOLM1 C9orf155 | Q8NBJ4 | GOLM1_HUMAN | Golgi membrane protein 1 (Golgi |
| GOLPH2 | | | membrane protein GP73) (Golgi |
| PSEC0242 | | | phosphoprotein 2) |
| UNQ686/PRO1326 | | | |
| MUC5AC MUC5 | P98088 | MUC5A_HUMAN | Mucin-5AC, MUC-5AC (Gastric |
| | | | mucin) (Major airway glycoprotein) |
| | | | (Mucin-5 subtype AC, tracheobronchial) |
| | | | (Tracheobronchial mucin, TBM) |
| NME1 NDPKA | P15531 | NDKA_HUMAN | Nucleoside diphosphate kinase A, |
| NM23 | | | NDK A, NDP kinase A, EC 2.7.4.6 |
| | | | (Granzyme A-activated DNase, |
| | | | GAAD) (Metastasis inhibition factor |
| | | | nm23) (NM23-H1) (Tumor metastatic |
| | | | process-associated protein) |
| NT5E NT5 NTE | P21589 | 5NTD_HUMAN | 5′-nucleotidase, 5′-NT, EC 3.1.3.5 |
| | | | (Ecto-5′-nucleotidase) (CD antigen |
| | | | CD73) |
| PDLIM5 ENH L9 | Q96HC4 | PDLI5_HUMAN | PDZ and LIM domain protein 5 |
| | | | (Enigma homolog) (Enigma-like PDZ |
| | | | and LIM domains protein) |
| RDX | P35241 | RADI_HUMAN | Radixin |
| VASP | P50552 | VASP_HUMAN | Vasodilator-stimulated phosphoprotein, VASP |

### Example 3

### ELISA-EC Diagnostic (MDK)

MDK is a good EC diagnostic biomarker when measured in both: mass spectrometry (MS) and antibody-based assay (ELISA technique). The levels of MDK in the cervical fluid of 241 patients (n=128 EC, 113 non-EC) shown statistical differences to distinguish between EC and non-EC patients, achieving AUC of 0.91 when measured by MS and 0.92 when measured by ELISA assay. Showing a correlation of 0.91 between the measurements of both approaches.

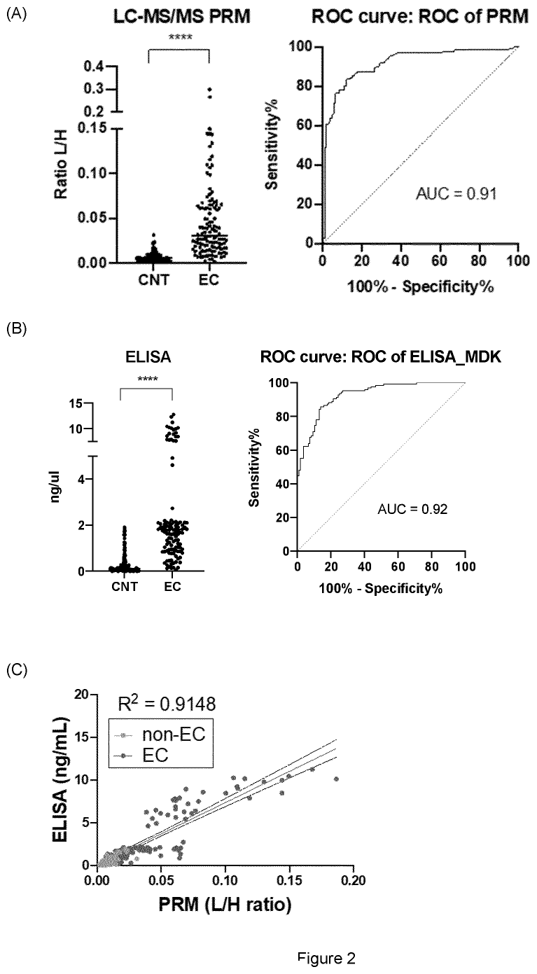

Data are depicted in FIG. 2, where in (A) mass spectrometry (LC-MS/MS PRM) data or in (B) an ELISA assay derived data for determining MDK, are illustrated. tested in samples of a cohort of subjects and comparing EC vs non-EC patients. In (C), the correlation between the measurements of both approaches, allowing to see the high correlation level, which makes the marker a one one for a point of care implementation.

### Example 4

### MDK in Uterine Fluids

The levels of MDK also demonstrated to be a potential diagnostic marker for EC in the uterine fluid obtained from endometrial biopsies, specifically, from pipelle biopsies. Specifically, MDK levels showed a p-value<0.0001 and AUC=0.69 between EC (n=118) and non-EC patients (n=129). Thus, besides the information from MDK that can be obtained from a sample type during routine gynecological inspection, this marker gives also information from other more invasive samplings, such as endometrial biopses. These biopses are sometimes done when suspection of certain diseases, so that also the analysis of the possibility of an incipient or not EC could be simultaneously detected if this marker is analysed.

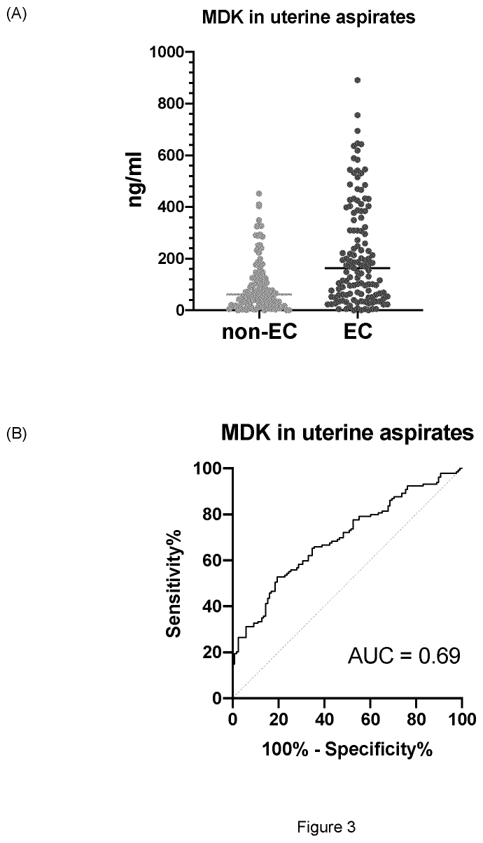

Data are depicted in FIG. 3, where data from an ELISA assay for determining MDK in uterine fluids, tested in samples of a cohort of subjects and comparing EC vs non-EC patients is illustrated. In (A) Dotplot of the distribution of protein concentration between EC and non-EC patients. In (B) the a curve of an ROC analysis (Receiver Operating Characteristic analysis) of MDK assessed by ELISA.

### Example 5

### Molecular Classification

A total number of 28 proteins (LBP, VWF, GPLD1, SAA4, APOF, C4BPA, SPRR1A, SERPIND1, APOB, SCEL, LCAT, SERPINA3, LMO7, C1R, MUC4, FN1, SPRR1B, C1QA, ITIH2, TIMP2, APOC1, GRN, ANXA3, S100A9, PLBD1, PIGR, SERPINH1, HSPE1) were able to significantly differentiate between molecular subgroups of EC: presence of POLE exonuclease domain hotspot mutation (POLE) ultramutated or POLE mutated (POLEmut), MSI: microsatellite instable (MSI) or MMR deficient, based on loss of mismatch repair protein expression (MMRd); Copy Number Low (CN-Low) or no specific molecular profile (NSMP); Copy Number High (CN-High) or p53 abnormal, based on mutant-like immunostaining (p53abn)-FC: Fold Change.

The data are depicted in Table 12:

Verification phase (n = 92)
Molecular classification

| | | | Adjusted | | Adjusted | | Adjusted |
|---|---|---|---|---|---|---|---|
| | | FC | p value | FC | p value | FC | p value |
| | | (MMRd | (MMRd | (MMRd | (MMRd | (NSMP | (NSMP |
| | Gene | vs | vs | vs p53 | vs p53 | vs | vs |
| Entry | name | NSMP) | NSMP) | mut) | mut) | p53mut) | p53mut) |
| P18428 | LBP | −1.64 | 0.04 | 1.33 | 0.85 | 2.19 | 0.02 |
| P04275 | VWF | −1.77 | 0.00 | 1.03 | 0.94 | 1.82 | 0.01 |
| P80108 | GPLD1 | −1.71 | 0.00 | −1.05 | 0.72 | 1.63 | 0.05 |
| P35542 | SAA4 | −1.64 | 0.01 | 1.14 | 0.98 | 1.86 | 0.07 |
| Q13790 | APOF | −1.87 | 0.00 | −1.16 | 0.88 | 1.61 | 0.07 |
| P04003 | C4BPA | −1.72 | 0.01 | −1.08 | 0.80 | 1.58 | 0.12 |
| P35321 | SPRR1A | 3.04 | 0.02 | −1.32 | 0.88 | −4.00 | 0.05 |
| P05546 | SERPIND1 | −1.78 | 0.00 | −1.14 | 0.88 | 1.56 | 0.07 |
| P04114 | APOB1 | −1.51 | 0.04 | 1.10 | 0.80 | 1.66 | 0.06 |
| O95171 | SCEL | 3.47 | 0.00 | 1.45 | 0.97 | −2.40 | 0.07 |
| P04180 | LCAT | −1.72 | 0.00 | −1.25 | 0.69 | 1.37 | 0.10 |
| P01011 | SERPINA3 | −1.96 | 0.00 | −1.32 | 0.61 | 1.48 | 0.13 |
| Q8WWI1 | LMO7 | 2.84 | 0.01 | 1.40 | 0.90 | −2.03 | 0.09 |
| P00736 | C1R | −1.52 | 0.02 | −1.19 | 0.70 | 1.29 | 0.13 |
| Q99102 | MUC4 | 1.74 | 0.04 | 1.03 | 0.94 | −1.70 | 0.14 |
| P02751 | FN1 | −1.36 | 0.01 | −1.07 | 0.72 | 1.28 | 0.10 |
| P22528 | SPRR1B | 2.10 | 0.01 | −1.65 | 0.84 | −3.47 | 0.10 |
| P02745 | C1QA | −1.33 | 0.02 | −1.16 | 0.61 | 1.15 | 0.19 |
| P19823 | ITIH2 | −1.61 | 0.00 | −1.24 | 0.59 | 1.30 | 0.13 |
| P16035 | TIMP2 | 1.54 | 0.04 | 1.02 | 0.94 | −1.51 | 0.12 |
| P02654 | APOC1 | −1.57 | 0.01 | −1.11 | 0.84 | 1.41 | 0.12 |
| P28799 | GRN | 2.20 | 0.00 | 1.44 | 0.35 | −1.53 | 0.12 |
| P12429 | ANXA3 | 2.15 | 0.00 | 1.44 | 0.41 | −1.49 | 0.18 |
| P06702 | S100A9 | 1.64 | 0.04 | 1.00 | 0.99 | −1.64 | 0.18 |
| Q6P4A8 | PLBD1 | 1.84 | 0.04 | 1.60 | 0.72 | −1.15 | 0.33 |
| P01833 | PIGR | 1.11 | 0.41 | −3.16 | 0.18 | −3.51 | 0.01 |
| P50454 | SERPINH1 | −1.08 | 0.32 | 1.99 | 0.14 | 2.14 | 0.00 |
| P61604 | HSPE1 | −1.32 | 0.05 | 1.04 | 0.89 | 1.37 | 0.05 |

Further aspects/embodiments are defined in the following clauses:

Clause 1. A method of diagnosis and/or for the prognosis of endometrial cancer, the method comprising determining the presence and/or the level of expression of midkine (MDK) in a sample from the female genital tract part including one or more of the vulvae, vagina, cervix, uterus, fallopian tubes, and ovaries, and selected from a gynecologic sampling, including or selected from a cervical fluid, a cytology, a pap-smear sample, an endometrial biopsy, a uterine fluid, uterine washings and combinations thereof.

Clause 2. The method according to clause 1, wherein the sample is a pap-smear, and specifically the fluid contained in a pap-smear and/or cervical fluid.

Clause 3. The method according to any of clauses 1-2, further comprising determining the presence and/or the level of expression of one or more of the proteins listed in Table 1, in particular, one or more of following proteins:
Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A) and Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Agrin (AGR), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1 (LGALS1), ATPase H+Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1(HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), and Exportin-2 (XPO2).

Clause 4. The method according to any of clauses 1-3, comprising determining the presence and/or the level of expression of two, three, or four of the proteins.

Clause 5. The method according to clause 4, comprising determining in the isolated sample the presence and/or the level of expression of the proteins in at least one binary set of the group consisting of: MDK, LCN2; MDK, FN1; MDK, PLBD1; MDK, APOB; MDK, CD44; MDK, FCGBP; MDK, EPS8L1; MDK, ANXA3; MDK, C4BPA; MDK, MMP8; MDK, NEDD-8; MDK, CAMP; MDK, TIMP2; MDK, HSPE1; MDK, CALU; MDK, C1QA; MDK, LDHA; MDK, TIMP2; MDK, PIGR; MDK, KRT8; MDK, PPL; MDK, SPRR1A; MDK, STMN1; MDK, CAPS; MDK, CA1; MDK, CCT6A; MDK, VIM; MDK, TCP1; and MDK, AGRN; and/or at least one binary set listed on any of Tables 4 and 6.

Clause 6. The method according to clause 4, comprising determining in the isolated sample the presence and/or the level of expression of the proteins in at least one ternary set of the group consisting of: MDK, ANXA7, CSE1L; MDK, LCN2, FGFBP1; MDK, LCN2, ANXA7; MDK, FGFBP1, LCN2; MDK, APOF, FCGBP; MDK, IMPA1, FCGBP; MDK, LCN2, CD44; MDK, STX7, FCGBP; MDK, ITIH2, FCGBP; MDK, LCN2, EPS8L1; MDK, FGFBP, EPS8L1; MDK, ANXA7, FCGBP; MDK, LCN2, PLBD1; MDK, FCGBP, CD44; MDK, PLBD1, FCGBP; MDK, ANXA3, FCGBP; MDK, PLBD1, LGALS1; MDK, LCN2, IMPA1; MDK, FCGBP, VIM; MDK, FCGBP, LMNB1; MDK, PLBD1, ATP6V1G1; MDK, APOB, PKM; MDK, ITIH2, LCN2; MDK, PLBD1, CALU; MDK, LCN2, APOF; MDK, PLBD1, EPS8L1; MDK, APOC1, ANXA7; MDK, FCGBP, GYG1; MDK, PLBD1, PKM; MDK, FCGBP, LSP1; MDK, TIMP2, CD44; MDK, ANXA3, PLBD1; MDK, PLBD1, HSPE1; MDK, PLBD1, CA1; MDK, FCGBP, HCLS1; MDK, STX7, IMPA1; MDK, ANXA7, STX7; MDK, STX7, EPS8L1; MDK, PLBD1, LSP1; MDK, C1R, ANXA7; MDK, STX7, PEA15; MDK, PLBD1, HCLS1; MDK, LCAT, FCGBP; MDK, PLBD1, LDHA; MDK, APOB, S100A9; MDK, ANXA7, SCEL; MDK, SERPINA3, FCGBP; MDK, FCGBP, ITGB2; MDK, SCEL, ANXA3; MDK, S100A9, PLBD1; MDK, S100A9, LSP1; MDK, C1R, FCGBP; MDK, FGFBP1, FCGBP; MDK, APOC1, S100A9; MDK, PPL, PLBD1; MDK, TCP1, PEA15; MDK, ANXA3, LSP1; MDK, PPL, IMPA1; MDK, S100A9, MMP8; MDK, FGFBP1, PPL; and MDK, S100A9, PPL; and or at least one ternary set listed on any of Tables 5 and 7.

Clause 7. The method according to clause 6, comprising determining in the isolated sample the presence and/or the level of expression of the ternary set MDK, ANXA7 and CSE1L

Clause 8. The method according to any of the clauses 1-7, comprising:
a) determining, in vitro, the level of expression of MDK, optionally in combination with one or more of the proteins listed in any one of claims 3-7, in the isolated sample from the genital tract of the female;
b) comparing the level of MDK and if determined of the one or more other the proteins of step (a) with a corresponding reference value or reference interval for each protein, said reference value or interval selected from a value or interval of values from a subject suffering from endometrial cancer, and/or comparing with a cut-off value discriminating among endometrial cancer and other endometriod disorders or conditions of (i.e., a healthy) endometrium, and wherein the subject is diagnosed of endometrial cancer if at least the level of MDK is within the value or interval of values from a subject suffering from this cancer, and/or if at least the level of MDK in relation to the cut-off value is classified to the endometrial cancer group.

Clause 9. The method according to any of clauses 1-8, wherein the level of expression is determined at the protein level.

Clause 10. The method according to any of clauses 1-9, wherein the protein level is determined by an assay or technology selected from the group consisting of an immunoassay, a bioluminescence assay, a fluorescence assay, a chemiluminescence assay, electrochemistry assay, mass spectrometry, and combinations thereof.

Clause 11. The method according to any of clauses 9-10, wherein the level of expression of protein is determined using an antibody or a fragment thereof able to bind to the protein.

Clause 12. The method according to clause 11, wherein said antibody or fragment o thereof forms part of a kit.

Clause 13. Use of MDK, as in vitro marker for the diagnosis and/or for the prognosis of endometrial cancer in a sample in a sample from the female genital tract part including one or more of the vulvae, vagina, cervix, uterus, fallopian tubes, and ovaries and selected from a gynecologic sampling, including one or more of a cervical fluid, a cytology, a pap-smear sample, an endometrial biopsy, a uterine fluid, uterine washings.

Clause 14. A kit comprising a solid support and means for detecting the presence and/or for determining the level of expression of MDK and optionally means for detecting the presence and/or for determining the level of expression of one or more proteins listed in Table 1, in particular one or more proteins selected from the group consisting of Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A) and Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Agrin (AGR), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1 (LGALS1), ATPase H+Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B) and Exportin-2 (XPO2).

Clause 15. A computer-implemented method for carrying out the method as defined in any of clauses 1-3, in which after the determination of the level of expression of MDK, and optionally of the one or more of the proteins for the diagnosis and/or for the prognosis of endometrial cancer, said level(s) are given a value and/or a score, and optionally are computed in a mathematical formula to obtain a computed value; wherein in function of the said level(s), score(s) and or computed value(s), a decision is taken between the options of suffering or not from EC and/or between the options of suffering among different EC presenting different prognosis, including different histological subtypes and grades and different molecular features.

### CITATION LIST

DeSouza LV, et al, “Endometrial cancer biomarker discovery and verification using differentially tagged clinical samples with multidimensional liquid chromatography and tandem mass spectrometry”, Mol Cell Proteomics MCP-2007, vol. no.6, pp.: 1170-8.
EP3452829B1
EP3655778A1
Tanable et al., “Midkine and its clinical significance in endometrial carcinoma”, cancer Sci-2008, vol. no. 99 (6), pp.: 1125-1130
Kemik P, et al. “Diagnostic and prognostic values of preoperative serum levels of YKL-40, HE-4 and DKK-3 in endometrial cancer”, Gynecol Oncol-2016; vol. no. 140, pp.: 64-9.
Burtis C. A. et al., 2008, Chapter 14, section “Statistical Treatment of Reference Values”

What is claimed is:

1.-19. (canceled)

20. A method of diagnosis and/or for the prognosis of endometrial cancer in a subject at risk, the method comprising:
a) obtaining an isolated sample from a female genital tract selected from the group consisting of one or more of the vulvae, vagina, cervix, uterus, fallopian tubes, and ovaries; and
b) determining a presence and/or level of expression of one or more proteins selected from the group consisting of Agrin (AGR), Midkine (MDK), Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A), Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1 (LGALS1), ATPase H+Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), Exportin-2 (XPO2), A2ML1 (Alpha-2-macroglobulin-like protein 1), APP (Amyloid-beta precursor protein), BTD (Biotinidase), CD44 (CD44 antigen), COL12A1 (Collagen alpha-1 (XII) chain), COL1A1 (Collagen alpha-1 (I) chain), COL3A1 (Collagen alpha-1 (III) chain), CTNNB1 (Catenin beta-1), FAM107B (Protein FAM107B), GPLD1 (Glycoprotein phospholipase D), GRN (Progranulin), IGFALS (Insulin-like growth factor-binding protein complex acid labile subunit), LMNB1 (Lamin-B1), LMO7 (LIM domain only protein 7), MUC4 (Mucin-4), NAMPT (Nicotinamide phosphoribosyltransferase), RAB21 (Ras-related protein), SERPINH1 (Serpin H1), SPP1 (Osteopontin), TMPRS11E (Transmembrane protease serine 11E), and VWF (von Willebrand factor);
wherein the presence of the one or more proteins and/or increased expression levels of the one or more proteins is indicative that the subject is at risk of having or developing endometrial cancer.

21. A method of treating endometrial cancer in a subject in need thereof, the method comprising carrying out the method of diagnosis and/or prognosis of claim 20; and further comprising treating the subject with a therapy appropriate for endometrial cancer.

22. The method according to claim 21, wherein the therapy is selected from the group consisting of total hysterectomy, bilateral salpingo-oophorectomy, and combinations thereof, optionally complemented with pelvic and para-aortic lymphadenectomy, and/or omentectomy.

23. The method according to claim 21, wherein the therapy further comprises an adjuvant therapy selected from the group consisting of radiotherapy, brachytherapy, hormone therapy, chemotherapy, targeted therapy, and combinations thereof.

24. The method according to claim 20, wherein the isolated sample is a fluid contained in a pap-smear and/or cervical fluid.

25. The method according to claim 20, comprising determining the presence and/or the expression level of two, three, or four of the one or more proteins.

26. The method according to claim 25, comprising determining in the isolated sample the presence and/or the expression level of the one or more proteins in at least one binary set listed on any of Tables 4 and 6.

27. The method according to claim 25, comprising determining in the isolated sample the presence and/or the expression level of the one or more proteins in at least one ternary set listed on any of Tables 5 and 7.

28. The method according to claim 27 comprising determining in the isolated sample the presence and/or the expression level of at least one binary set selected from the group consisting of AGRN and LCN2, AGRN and PLBD1, AGRN and GYG1, AGRN and FCGBP, AGRN and ANXA3, AGRN and GRN, AGRN and TIMP2, AGRN and ITGB2, AGRN and A2ML1, AGRN and SPRR1A, AGRN and TMPRSS11E, AGRN and GYG1, AGRN and COL1A1, AGRN and SPP1, and AGRN and APP.

29. The method according to claim 20, comprising:
a) determining, in vitro, the expression level of one or more of the following proteins: AGRIN, MDK, APOB, C1QA, FN1, SERPIND1, APOF, APOC1, CCT6A, LBP, SAA4, ITIH2, LCN2, LCAT, C4BPA, C1R, FGFBP1, SPRR1B, SPRR1A, TIMP2, LCN2, PLBD1, CD44 antigen, FCGBP, EPS8L1, ANXA3, MMP8, NEDD-8 protein, CAMP, HSPE1, CALU, LDHA, PIGR, KRT8, PPL, STMN1, CAPS, CA1, VIM, TCP1, ANXA7, IMPA1, STX7, ITIH2, LGALS1, ATP6V1G1, PKM, GYG1, LSP1, HCLS1, PEA15, S100A9, SCEL, SERPINA3, ITGB2, FCGBP, NEDD8-MDP1, CHMP4B, XPO2, A2ML1, APP, BTD, CD44, COL12A1, COL1A1, COL3A1, CTNNB1, FAM107B, GPLD1, GRN, IGFALS, LMNB1, LMO7, MUC4, NAMPT, RAB21, SERPINH1, SPP1, TMPRS11E, and VWF, in an isolated sample obtained from the genital tract of the female;
b) comparing the expression level of the one or more of the proteins of step (a) with a corresponding reference value or reference interval for each protein, said reference value or interval, wherein the reference value or interval is selected from a value or interval of values from a subject suffering from endometrial cancer, and/or comparing reference value or reference interval for each protein with a cut-off value determined relative to a group of subjects suffering from endometrial cancer, wherein the cut-off value discriminates among endometrial cancer and other endometriod disorders or conditions of a healthy endometrium; and
wherein the subject is diagnosed with endometrial cancer if either the expression level of the one or more of the proteins of step (a) is within the corresponding reference value or reference interval of values from a subject suffering from endometrial cancer, and/or if the expression level of the one or more of the proteins of step (a) in relation to the corresponding cut-off value is within a range of a group of subjects suffering from endometrial cancer.

30. The method according to claim 20, wherein the diagnosis is for recurrence or risk of recurrence of endometrial cancer, which further comprises determining in the isolated sample the presence and/or the expression level of one or more of the proteins selected from the group consisting of: MUC1, PRSS8, PNP, APEH, MUC16, C9, SERPINC1, SERPINA1, F2, AMBP, HP, SERPINA3, CFB, ORM2, CAT, GNAI2, A1BG, FN1, C7. ASTRGL1, B4GALT1, CAPS, CBX3, CD163, CDV3, DMBT1, DSG3, EHD1, GOLM1, MUC5AC, NME1, NT5E, PDLIM5, RDX, and VASP.

31. The method according to claim 20, wherein the diagnosis is for endometrial cancer subtype, and which further comprises determining in the isolated sample the presence and/or the expression level of one or more of the proteins selected from the group consisting of: LBP, VWF, GPLD1, SAA4, APOF, C4BPA, SPRR1A, SERPIND1, APOB, SCEL, LCAT, SERPINA3, LMO7, C1R, MUC4, FN1, SPRR1B, C1QA, ITIH2, TIMP2, APOC1, GRN, ANXA3, S100A9, PLBD1, PIGR, SERPINH1, HSPE1

32. The method according to claim 20, wherein the expression level is determined at the protein level.

33. The method according to claim 32, wherein the expression level is determined by an assay selected from the group consisting of an immunoassay, a bioluminescence assay, a fluorescence assay, a chemiluminescence assay, electrochemistry assay, mass spectrometry, and combinations thereof.

34. The method according to claim 32, wherein the expression level of protein is determined using an antibody or a fragment thereof able to bind to the protein.

35. A computer-implemented method for carrying out the method as defined in claim 20, in which after the determination of the expression level of the one or more proteins for the diagnosis and/or for the prognosis of endometrial cancer, said level(s) are given a value and/or a score, and optionally are computed in a mathematical formula to obtain a computed value;
wherein in function of the said level(s), score(s) and or computed value(s), a decision is taken between the options of suffering or not from endometrial cancer and/or between the options of suffering among different endometrial cancers presenting different prognosis, including different histological subtypes and grades and different molecular features.

36. A method of diagnosis and/or for the prognosis of endometrial cancer in a subject at risk, the method comprising:
a) providing an antibody or fragment thereof able to bind to a protein of interest to an isolated sample obtained from a female genital tract; and
b) determining, in vitro, a presence and/or level of expression of one or more proteins selected from the group consisting of Agrin (AGR), Midkine (MDK), Apolipoprotein B (APOB), Complement C1q subcomponent subunit A (C1QA), Fibronectin 1 (FN1), Serpin Family D Member 1 (SERPIND1), apolipoprotein F precursor (APOF), Apolipoprotein C1 (APOC1), Chaperonin Containing TCP1 Subunit 6A (CCT6A), lipopolysaccharide-binding protein precursor (LBP), Serum Amyloid A4 (SAA4), Inter-Alpha-Trypsin Inhibitor Heavy Chain 2 (ITIH2), Lipocalin 2 (LCN2), Lecithin: cholesterol acyltransferase (LCAT), C4b-binding protein alpha chain (C4BPA), Complement C1r (C1R), Fibroblast Growth Factor Binding Protein 1 (FGFBP1), Small Proline Rich Protein 1B (SPRR1B), Small Proline Rich Protein 1A (SPRR1A), Tissue inhibitor of metalloproteinases 2 (TIMP2), Liopocalin-2 (LCN2), Phospholipase B Domain Containing 1 (PLBD1), CD44 antigen, Fc Fragment Of IgG Binding Protein (FCGBP), Epidermal growth factor receptor kinase substrate 8-like protein 1 (EPS8L1), Annexin A3 (ANXA3), matrix metalloproteinase-8 (MMP8), NEDD-8 protein, Cathelicidin Antimicrobial Peptide (CAMP), Heat Shock Protein Family E (Hsp10) Member 1 (HSPE1), Calumenin (CALU), Lactate Dehydrogenase A (LDHA), Polymeric Immunoglobulin Receptor (PIGR), Keratin 8 (KRT8), Periplakin (PPL), Stathmin 1 (STMN1), Calcyphosin (CAPS), Carbonic anhydrase 1 (CA1), Vimentin (VIM), T complex 1 (TCP1), Annexin A7 (ANXA7), Inositol Monophosphatase 1 (IMPA1), Syntaxin 7 (STX7), Inter-Alpha-Trypsin Inhibitor Heavy Chain 2 (ITIH2), Galectin 1 (LGALS1), ATPase H+Transporting V1 Subunit G1 (ATP6V1G1), Pyruvate kinase isozymes M1/M2 (PKM), Glycogenin 1 (GYG1), Lymphocyte-specific protein 1 (LSP1), Hematopoietic Cell-Specific Lyn Substrate 1 (HCLS1), Proliferation And Apoptosis Adaptor Protein 15 (PEA15), S100 calcium-binding protein A9 (S100A9), Sciellin (SCEL), Serpin Family A Member 3 (SERPINA3), Integrin Subunit Beta 2 (ITGB2), Fc Fragment Of IgG Binding Protein (FCGBP), NEDD8-MDP1 protein (NEDD8-MDP1), Charged Multivesicular Body Protein 4B (CHMP4B), Exportin-2 (XPO2), A2ML1 (Alpha-2-macroglobulin-like protein 1), APP (Amyloid-beta precursor protein), BTD (Biotinidase), CD44 (CD44 antigen), COL12A1 (Collagen alpha-1 (XII) chain), COL1A1 (Collagen alpha-1 (l) chain), COL3A1 (Collagen alpha-1 (III) chain), CTNNB1 (Catenin beta-1), FAM107B (Protein FAM107B), GPLD1 (Glycoprotein phospholipase D), GRN (Progranulin), IGFALS (Insulin-like growth factor-binding protein complex acid labile subunit), LMNB1 (Lamin-B1), LMO7 (LIM domain only protein 7), MUC4 (Mucin-4), NAMPT (Nicotinamide phosphoribosyltransferase), RAB21 (Ras-related protein), SERPINH1 (Serpin H1), SPP1 (Osteopontin), TMPRS11E (Transmembrane protease serine 11E), and VWF (von Willebrand factor);
wherein the presence of the one or more proteins and/or increased expression levels of the one or more proteins is indicative that the subject is at risk of having or developing endometrial cancer.

37. The method of claim 36, wherein the antibody or fragment thereof is attached directly or indirectly to a solid support.

38. The method according to claim 36, wherein the isolated sample is a fluid contained in a pap-smear and/or cervical fluid.

39. The method according to claim 36, comprising determining the presence and/or the expression level of two, three, or four of the one or more proteins.
